(19) United States
(12) Patent Application Publication
(10) Pub. No.: US 2012/0094897 A1
(43) Pub. Date: Apr. 19, 2012

(54) NOVEL INHIBITORS OF HEPATITIS C VIRUS REPLICATION

(75) Inventors: Brad Buckman, Oakland, CA (US); John B. Nicholas, Redwood City, CA (US); Vladimir Serebryany, Burlingame, CA (US); Scott D. Seiwert, Pacifica, CA (US)

(73) Assignee: InterMune, Inc., Brisbane, CA (US)

(21) Appl. No.: 13/232,815

(22) Filed: Sep. 14, 2011

Related U.S. Application Data
(60) Provisional application No. 61/383,220, filed on Sep. 15, 2010.
(60) Provisional application No. 61/473,608, filed on Apr. 8, 2011.

(51) Int. Cl. A61K 38/06; A61P 31/14; A61P 1/16; C07K 5/093; C07K 5/083; G21C 3/00

(52) U.S. Cl. 514/4.3; 530/331

(57) ABSTRACT

The embodiments provide compounds of the general Formulae I, and II, as well as compositions, including pharmaceutical compositions, comprising a subject compound. The embodiments further provide treatment methods, including methods of treating a hepatitis C virus infection and methods of treating liver fibrosis, the methods generally involving administering to an individual in need thereof an effective amount of a subject compound or composition.

### RELATED APPLICATIONS

This application claims the benefit of U.S. Provisional Application Nos. 61/383,220, filed Sep. 15, 2010; and 61/473,608, filed Apr. 8, 2011; the disclosures of which are incorporated herein by reference in their entirety.

### BACKGROUND

1. Field of the Invention

The present invention relates to compounds, processes for their synthesis, compositions and methods for the treatment of hepatitis C virus (HCV) infection.

2. Description of the Related Art

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy, i.e., are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

HCV is an enveloped positive strand RNA virus in the Flaviviridae family. The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins of the virus. In the case of HCV, the generation of mature nonstructural proteins (NS2, NS3, NS4, NS4A, NS4B, NS5A, and NS5B) is effected by two viral proteases. The first viral protease cleaves at the NS2-NS3 junction of the polyprotein. The second viral protease is serine protease contained within the N-terminal region of NS3 (herein referred to as “NS3 protease”). NS3 protease mediates all of the subsequent cleavage events at sites downstream relative to the position of NS3 in the polyprotein (i.e., sites located between the C-terminus of NS3 and the C-terminus of the polyprotein). NS3 protease exhibits activity both in cis, at the NS3-NS4 cleavage site, and in trans, for the remaining NS4A-NS4B, NS4B-NS5A, and NS5A-NS5B sites. The NS4A protein is believed to serve multiple functions, acting as a cofactor for the NS3 protease and possibly assisting in the membrane localization of NS3 and other viral replicase components. Apparently, the formation of the complex between NS3 and NS4A is necessary for NS3-mediated processing events and enhances proteolytic efficiency at all sites recognized by NS3. The NS3 protease also exhibits nucleoside triphosphatase and RNA helicase activities. NS5B is an RNA-dependent RNA polymerase involved in the replication of HCV RNA. In addition, compounds that inhibit the action of NS5A in viral replication are potentially useful for the treatment of HCV.

### SUMMARY

Some embodiments provide a compound represented by Formula I:

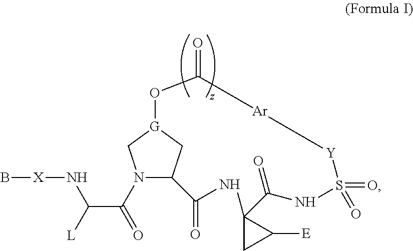

or a pharmaceutically acceptable salt thereof, wherein Ar is optionally substituted heteroaryl, optionally substituted C₆₋₁₀ aryl, optionally substituted heterocyclyl; or optionally substituted

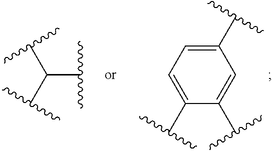

polycyclic moiety; z is 0 or 1; G is X is a bond, CO, CO₂, CONH, SO₂, SO₃, or SO₂NH; B is H (hydrogen), optionally substituted C₆₋₁₀ aryl, optionally substituted C₂₋₁₀ heteroaryl, or optionally substituted C₁₋₁₀ hydrocarbyl; L is H (hydrogen) or C₁₋₁₀ hydrocarbyl.

Y is (L₁)ₚ; wherein p is an integer from 5 to 12; each L₁ is separately selected, where L₁ is selected from the group consisting of C(R²)₂, NR³, O (oxygen), —(R²)C═C(R²)—, C(═O), C₃₋₇ cycloalkyl, optionally substituted aryl, optionally substituted polycyclic moiety, optionally substituted heterocycle and optionally substituted heteroaryl. Each R² is separately selected, where R² is selected from the group consisting of H (hydrogen), C₁₋₆alkoxy, aryl, halo, hydroxy, R^{a}R^{b}N—, C₁₋₆alkyl optionally substituted with up to 5 halo, and C₁₋₆alkoxy optionally substituted with up to 5 halo, or optionally two vicinal R² and the carbons to which they are attached are together a fused three- to six-membered carbocyclic ring optionally substituted with up to two C₁₋₆alkyl groups, or optionally two geminal R² and the carbon to which they are attached are together a fused three- to six-membered carbocyclic ring optionally substituted with up to two C₁₋₆alkyl groups; each R^{a}R^{b}N is separately selected, wherein R^{a} and R^{b} are each separately selected from the group consisting of hydrogen, C₂₋₆alkenyl, and C₁₋₆alkyl.

Each R³ is separately selected, where R³ is selected from the group consisting of hydrogen, optionally substituted aryl, optionally substituted heteroaryl, and optionally substituted C₁₋₆alkyl; and E is H (hydrogen) or optionally substituted C₁₋₆ hydrocarbyl.

In some embodiments, a compound represented by formula I is not selected from the group consisting of:

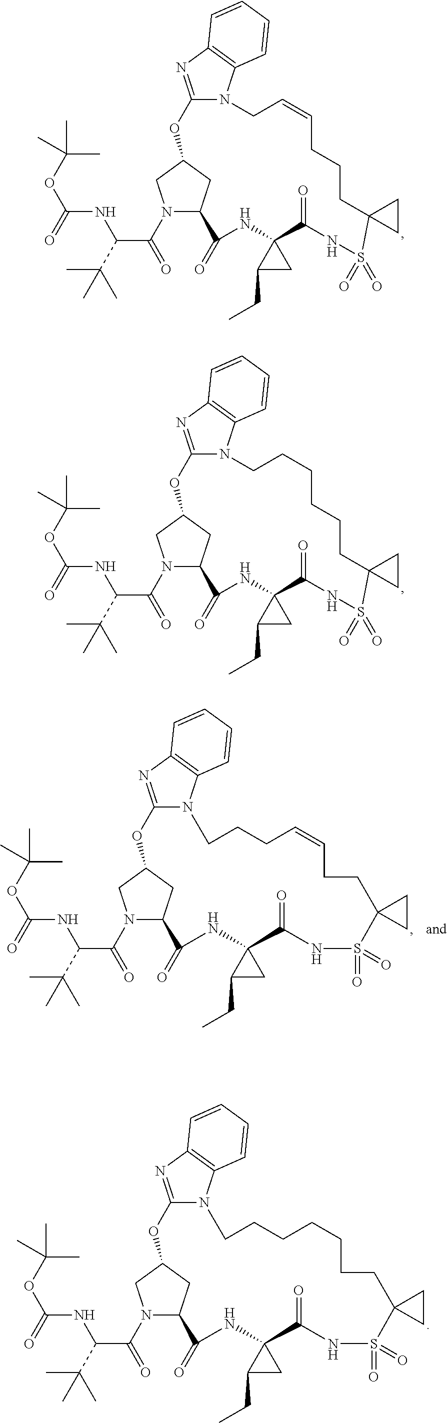

These definitions of Ar, z, G, B, X, L, Y, and E are understood to apply to structures depicted herein for which any one of those variables are not expressly defined.

Some embodiments provide a compound represented by Formula II:

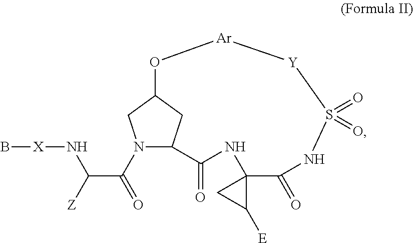

or a pharmaceutically acceptable salt thereof, wherein Ar is optionally substituted C₅₋₁₀ fused bicyclic heteroaryl, optionally substituted C_{6 or 10} aryl; or optionally substituted polycyclic moiety.

Y is (L₁)ₚ; p is an integer from 5 to 9; each L₁ is separately selected, where L₁ is selected from the group consisting of C(R²)₂, NR³, O (oxygen), —(R²)C═C(R²)—, C(═O), C₃₋₇ cycloalkyl, optionally substituted aryl, optionally substituted polycyclic moiety, optionally substituted heterocycle and optionally substituted heteroaryl; each R² is separately selected, where R² is selected from the group consisting of H (hydrogen), C₁₋₆alkoxy, C₁₋₆alkyl, aryl, halo, hydroxy, R^{a}R^{b}N—,C₁₋₆alkyl optionally substituted with up to 5 halo, and C₁₋₆alkoxy optionally substituted with up to 5 halo, or optionally two vicinal R² and the carbons to which they are attached are together a fused three- to six-membered carbocyclic ring optionally substituted with up to two C₁₋₆alkyl groups, or optionally two geminal R² and the carbons to which they are attached are together a fused three- to six-membered carbocyclic ring optionally substituted with up to two C₁₋₆alkyl groups; each R^{a}R^{b}N is separately selected, wherein R^{a} and R^{b} are each separately selected from the group consisting of hydrogen, C₂₋₆alkenyl, and C₁₋₆alkyl.

Each R³ is separately selected, where R³ is selected from the group consisting of hydrogen, optionally substituted aryl, optionally substituted heteroaryl, optionally substituted C₁₋₆alkyl, and C₁₋₆alkyl optionally substituted with up to 5 halo.

X is a bond, C(═O), —C(═O)O—, —C(═O)NH—, SO₂, SO₃, or SO₂NH.

B is H (hydrogen), optionally substituted C₁₋₆ alkyl, optionally substituted C₂₋₆ alkenyl, optionally substituted C₂₋₆ alkynyl, optionally substituted C₃₋₇ cycloalkyl, optionally substituted C_{6 or 10} aryl, optionally substituted C₅₋₁₀ heteroaryl, or optionally substituted C₅₋₁₀ heterocycle.

Z is H (hydrogen) or optionally substituted C₁₋₁₀ hydrocarbyl; and E is H (hydrogen) or optionally substituted C₁₋₆ hydrocarbyl.

Some embodiments provide a method of inhibiting NS3/NS4 protease activity comprising contacting a NS3/NS4 protease with a compound disclosed herein.

Some embodiments provide a method of treating hepatitis by modulating NS3/NS4 protease comprising contacting a NS3/NS4 protease with a compound disclosed herein.

Some embodiments provide a pharmaceutical composition comprising: a) a compound disclosed herein; and b) a pharmaceutically acceptable carrier.

Some embodiments provide a method of treating a hepatitis C virus infection in an individual, the method comprising administering to the individual an effective amount of a composition comprising a compound disclosed herein.

Some embodiments provide a method of treating liver fibrosis in an individual, the method comprising administering to the individual an effective amount of a composition comprising a compound disclosed herein.

Some embodiments provide a method of increasing liver function in an individual having a hepatitis C virus infection, the method comprising administering to the individual an effective amount of a composition comprising a compound disclosed herein.

These and other embodiments are described in greater detail below.

### DETAILED DESCRIPTION

### Definitions

The terms “individual,” “host,” “subject,” and “patient” are used interchangeably herein, and refer to a mammal, including, but not limited to, murines, primates, including simians and humans, mammalian farm animals, mammalian sport animals, and mammalian pets.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

“Treat,” “treating,” “treatment,” or another form thereof refers to the use of a compound, composition, therapeutically active agent, or drug in the diagnosis, cure, mitigation, treatment, or prevention of disease or other undesirable condition in a mammal; or the use of a compound, composition, therapeutically active agent, or drug in a manner intended to affect the structure or any function of the body of a mammal.

The term “optionally substituted,” as used herein refers to a moiety or structural feature which may be unsubstituted, or may have one or more substituents. Thus, for example, “optionally substituted phenyl” may be unsubstituted phenyl, or may be phenyl with one or more substituents. The term “substituent” as used herein refers to a moiety that replaces one or more hydrogen atoms of the parent group for which it is a substituent. In some embodiments, a substituent consists of from 0-10 carbon atoms, from 0-26 hydrogen atoms, from 0-5 oxygen atoms, from 0-5 nitrogen atoms, from 0-5 sulfur atoms, from 0-7 fluorine atoms, from 0-3 chlorine atoms, from 0-3 bromine atoms, and/or from 0-3 iodine atoms. In some embodiments, a substituent may comprise at least one carbon atom or one heteroatom selected from N (nitrogen), O (oxygen), and S (sulfur), and may comprise 0-12 carbon atom, 0-6 carbon atoms, or 0-3 carbon atoms, and 0-12 heteroatoms, 0-6 heteroatoms, 0-3 heteroatoms, or 1 heteroatom. Examples include C₁-C₆ alkyl, C₁-C₆ alkenyl, C₁-C₆ alkynyl, C₃-C₆ cycloalkyl, C₃-C₆ heterocycloalkyl (e.g., tetrahydrofuryl), aryl, heteroaryl, halo (e.g., chloro, bromo, iodo and fluoro), cyano, hydroxy, C₁-C₆ alkoxy, aryloxy, sulfhydryl (mercapto), C₁-C₆ alkylthio, arylthio, mono-(C₁-C₆)alkylamino (e.g. —NHMe), di-(C₁-C₆)alkylamino (e.g. —NMe₂), quaternary ammonium salts, amino(C₁-C₆)alkoxy, hydroxy(C₁-C₆)alkylamino, amino(C₁-C₆)alkylthio, cyanoamino, nitro, carbamyl, keto (oxo), carboxy, glycolyl, glycyl, hydrazino, guanyl, sulfamyl, sulfonyl, sulfinyl, thiocarbonyl, thiocarboxy, and combinations thereof. The protecting groups that can form the protective derivatives of the above substituents are known to those of skill in the art and can be found in references such as Greene and Wuts Protective Groups in Organic Synthesis; John Wiley and Sons: New York, 1999.

The term “aryl” as used herein refers to an aromatic ring or aromatic ring system such as phenyl, naphthyl, biphenyl, and the like. A phrase such as “C₆₋₁₀ aryl” as used herein refers to the number of carbon atoms in the ring or ring system (i.e. 6-10), but does not characterize or limit any substituents of the aryl moiety. Other similar numerical designations such as “C₁₋₁₀” have analogous meanings and may be applied to any type of moiety, such as “hydrocarbyl,” “alkyl,” “alkyl ether,” etc.

The term “heteroaryl” as used herein refers to an aromatic ring or aromatic ring system having one or more oxygen atom, nitrogen atom, sulfur atom, or a combination thereof, which are part of the ring or ring system. Examples include thienyl, furyl, quinolinyl, thiazolyl, benzooxazolyl, benzothiazolyl, benzoimidazolyl, benzothienyl, benzofuryl, pyridinyl, imidazolyl, thiazolyl, oxazolyl, and the like. The term “fused bicyclic heteroaryl” as used herein refers to heteroaryl having a ring system of two rings, wherein two adjacent ring atoms are shared by both rings of the system.

The term “heterocyclic” or “heterocyclyl” or “heterocycloalkyl” used herein refers to cyclic non-aromatic ring system radical having at least one ring in which one or more ring atoms are not carbon, namely heteroatom. The heteroatoms are independently selected from oxygen, sulfur, and nitrogen. In fused ring systems, the one or more heteroatoms may be present in only one of the rings and each ring in the fused system is non-aromatic. Preferred monocyclic ring systems are of 4, 5, 6, 7, or 8 members. Six membered monocyclic rings preferably contain from one to three heteroatoms wherein each heteroatom is individually selected from oxygen, sulfur, and nitrogen. Five-membered rings preferably have one or two heteroatoms wherein each heteroatom is individually selected from oxygen, sulfur, and nitrogen. Examples of heterocyclic groups include, but are not limited to, morpholinyl, tetrahydrofuranyl, dioxolanyl, pyrrolidinyl, pyranyl, piperidyl, piperazyl, and the like.

The term “polycyclic moiety” used herein refers to an optionally substituted bicyclic or tricyclic ring system comprising at least one heteroatom in the ring system backbone, wherein at least one ring is aromatic and at least one ring is non-aromatic. The heteroatoms are independently selected from oxygen, sulfur, and nitrogen. The term, “polycyclic moiety” includes multiple fused ring systems including, but not limited to, isoindolinyl, tetrahydroisoquinolinyl tetrahydroquinolinyl, and tetrahydroquinazolinyl. In some embodiments, the bicyclic or tricyclic ring system may be substituted or unsubstituted, and can be attached to other groups via any available valence, preferably any available carbon or nitrogen. Preferred bicyclic cyclic ring systems are of 8 to 12 members and include spirocycles. The bicyclic moiety contains two rings wherein the rings are fused. The bicyclic moiety can be appended at any position of the two rings. For example, bicyclic moiety may refer to a radical including but not limited to:

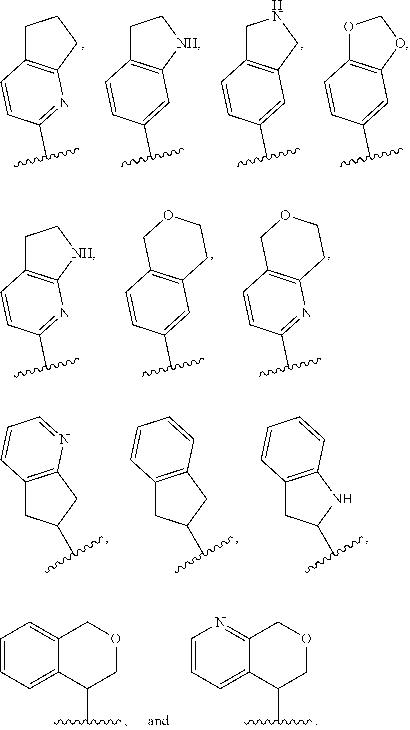

The tricyclic moiety contains a bicyclic moiety with an additional fused ring. The tricyclic moiety can be appended at any position of the three rings. For example, tricyclic moiety may refer to a radical including but not limited to:

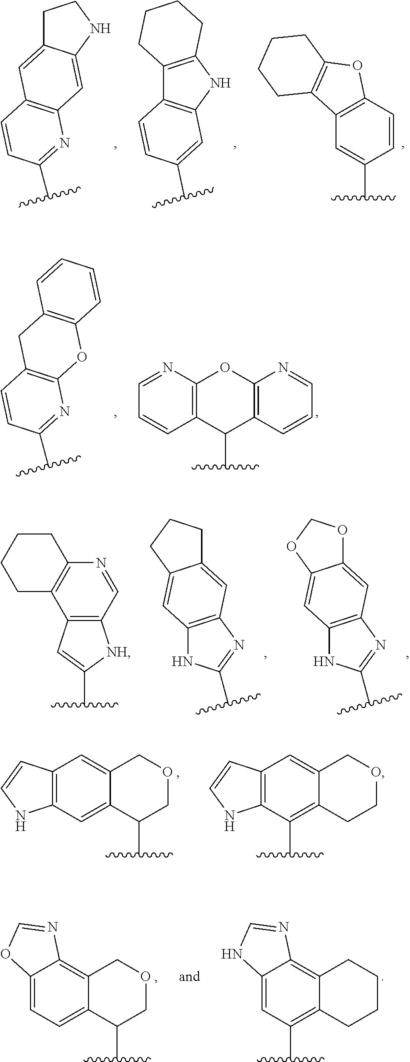

The term “hydrocarbyl” refers to an alkyl, cycloalkyl, alkenyl, cycloalkenyl or alkynyl moiety. The term “C₁₋₁₀ hydrocarbyl” refers to hydrocarbyl having 1, 2, 3, 4, 5, 6, 7, 8, 9, or 10 carbon atoms. The term “C₁₋₆ hydrocarbyl” refers to hydrocarbyl having 1, 2, 3, 4, 5, or 6 carbon atoms. The term “C₄₋₆ hydrocarbyl” refers to hydrocarbyl having 4, 5, or 6 carbon atoms.

The term “alkyl” refers to a branched or unbranched fully saturated acyclic aliphatic hydrocarbon group (i.e. composed of carbon and hydrogen containing no double or triple bonds). In some embodiments, alkyls may be substituted or unsubstituted. Alkyls include, but are not limited to, methyl, ethyl, propyl, isopropyl, butyl, isobutyl, tertiary butyl, pentyl, hexyl, and the like, each of which may be optionally substituted in some embodiments.

The term “alkenyl” used herein refers to a monovalent straight or branched chain radical of from two to twenty carbon atoms containing a carbon double bond including, but not limited to, 1-propenyl, 2-propenyl, 2-methyl-1-propenyl, 1-butenyl, 2-butenyl, and the like.

The term “alkynyl” used herein refers to a monovalent straight or branched chain radical of from two to twenty carbon atoms containing a carbon triple bond including, but not limited to, 1-propynyl, 1-butynyl, 2-butynyl, and the like.

The term “cycloalkyl” used herein refers to fully saturated aliphatic ring system radical having three to twenty carbon atoms including, but not limited to, cyclopropyl, cyclopentyl, cyclohexyl, cycloheptyl, and the like.

The term “cycloalkenyl” used herein refers to aliphatic ring system radical having three to twenty carbon atoms with one or two carbon-carbon double bond(s) in the ring. Examples of cycloalkenyl groups include, but are not limited to, cyclopropenyl, cyclopentenyl, cyclohexenyl, cycloheptenyl, and the like.

“Isoindolinyl” refers to the basic ring structure below. Attachment to the rest of the molecule or the addition of a substituent may occur at any possible position.

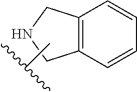

“Benzoimidazolen-1,2-yl” refers to the basic ring structure below. Addition of a substituent may occur at any possible position.

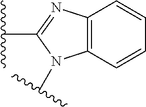

Asymmetric carbon atoms may be present in the compounds described. All such stereoisomers, both in a pure form or as a mixture of isomers, are intended to be included in the scope of a recited compound. In certain cases, compounds can exist in tautomeric forms. All tautomeric forms are intended to be included in the scope. Likewise, when compounds contain a double bond, there exists the possibility of cis- and trans-type isomeric forms of the compounds. Both cis- and trans-isomers, both in pure form as well as mixtures of cis- and trans-isomers, are contemplated. Thus, reference herein to a compound includes all of the aforementioned isomeric forms unless the context clearly dictates otherwise.

Alternate forms, including alternate solid forms, are included in the embodiments. Alternate solid forms such as polymorphs, solvates, hydrates, and the like, are alternate forms of a chemical entity that involve at least one of: differences in solid packing arrangements, non-covalent interactions with another compound such as water or a solvent. Salts involve at least one ionic interaction between an ionic form of a chemical entity of interest and a counter-ion bearing an opposite charge. Salts of compounds can be prepared by methods known to those skilled in the art. For example, salts of compounds can be prepared by reacting the appropriate base or acid with a stoichiometric equivalent of the compound. A prodrug is a compound that undergoes biotransformation (chemical conversion) to a parent compound (such as a compound described herein) in the body of an animal. Thus, reference herein to a compound includes all of the aforementioned forms unless the context clearly dictates otherwise.

The term “pharmaceutically acceptable salt,” as used herein, and particularly when referring to a pharmaceutically acceptable salt of a compound, including a compound of Formulas I or II, refers to any pharmaceutically acceptable salts of a compound, and preferably refers to an acid addition salt of a compound. With respect to compounds that contain a basic nitrogen, the preferred examples of pharmaceutically acceptable salts are acid addition salts of pharmaceutically acceptable inorganic or organic acids, for example, hydrohalic, sulfuric, phosphoric acid or aliphatic or aromatic carboxylic or sulfonic acid. Examples of pharmaceutically acceptable inorganic or organic acids as a component of an addition salt, include but are not limited to, hydrochloric acid, hydrobromic acid, phosphoric acid, sulfuric acid acetic acid, succinic acid, lactic acid, malic acid, tartaric acid, citric acid, ascorbi acid c, nicotinic acid, methanesulfonic acid, p-toluensulfonic acid or naphthalenesulfonic acid. With respect to compounds that contain an acidic functional group, the preferred examples of pharmaceutically acceptable salts include, but are not limited to, alkali metal salts (sodium or potassium), alkaline earth metal salts (calcium or magnesium), or ammonium salts derived from ammonia or from pharmaceutically acceptable organic amines, for example C₁-C₇ alkylamine, cyclohexylamine, triethanolamine, ethylenediamine or tris-(hydroxymethyl)-aminomethane.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Wherever a substituent as depicted as a di-radical (i.e., has two points of attachment to the rest of the molecule), it is to be understood that the substituent can be attached in any directional configuration unless otherwise indicated. Thus, for example, a substituent depicted as -AE- or

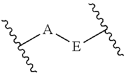

includes the substituent being oriented such that the A is attached at the leftmost attachment point of the molecule as well as attached at the rightmost attachment point of the molecule.

It is to be understood that certain radical naming conventions can include either a mono-radical or a di-radical, depending on the context. For example, where a substituent requires two points of attachment to the rest of the molecule, it is understood that the substituent is a di-radical. A substituent identified as alkyl, that requires two points of attachment, includes di-radicals such as —CH₂—, —CH₂CH₂—, —CH₂CH(CH₃)CH₂—, and the like; a substituent depicted as alkoxy that requires two points of attachment, includes di-radicals such as —OCH₂—, —OCH₂CH₂—, —OCH₂CH(CH₃)CH₂—, and the like: and a substituent depicted as arylC(═O)— that requires two points of attachment, includes di-radicals such as and the like.

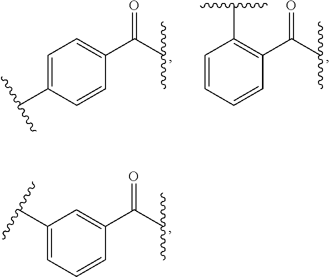

Where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the embodiments.

Unless defined otherwise, all technical and scientific terms used herein have the same meaning as commonly understood by one of ordinary skill in the art to which the embodiments belong. Although any methods and materials similar or equivalent to those described herein can also be used in the practice or testing of the embodiments, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

As employed herein, the following terms have their accepted meaning in the chemical literature.
anhyd. anhydrous
aq. aqueous
Boc tert-Butoxycarbonyl
Bu n-Butyl
Cat. Catalytic
CDI carbonyldiimidazole
° C. Temperature in degrees Centigrade
DBU 1,8-Diazabicyclo[5.4.0]undec-7-ene
DCE 1,2-dichloroethane
DCM dichloromethane
DIPEA Diisopropylethylamine
DMAP 4-Dimethylaminopyridine
DMF N,N′-Dimethylformamide
DMSO Dimethyl sulfoxide
eq. equivalents
g grams
HATU N,N,N′,N′-Tetramethyl-O-(7-azabenzotriazol-1-yl)uronium hexafluorophosphate
HPLC High-performance liquid chromatography (or high-pressure liquid chromatography
LC-MS Liquid chromatography-Mass spectrometry
LDA lithium diisopropylamide
M Molar
MeOH Methanol
mg Milligrams
MHz Megahertz
mL Milliliters
mM Millimolar
mmol Millimoles
nM Nanomolar
NMR Nuclear magnetic resonance
ppm parts-per-million
t Tert
t-Bu tert-Butyl
t-BuLi tert-Butyllithium
Tert tertiary
TFA trifluoroacetic acid
THF Tetrahydrofuran
t_{R} Retention time
UV Ultraviolet
W watt
μL Microliters
μM Micromolar

### Compounds

Unless otherwise indicated, if a term is used to describe more than one structural feature of the compounds disclosed herein, it should be assumed that the term has the same meaning for all of those features. Similarly, a subgroup of that term applies to every structural feature described by that term.

Some embodiments provide a compound represented by formula I:

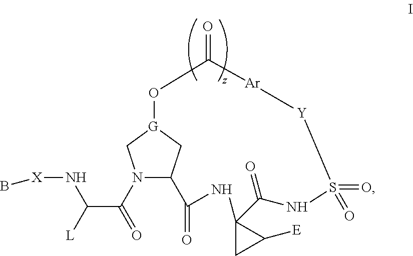

or a pharmaceutically acceptable salt thereof, wherein Ar is optionally substituted heteroaryl, optionally substituted C₆₋₁₀ aryl, optionally substituted heterocyclyl; or optionally substituted polycyclic moiety.

With respect to Formula I, in some embodiments, Ar may be an optionally substituted C₅₋₁₀ fused bicyclic heteroaryl. In some embodiments, Ar is optionally substituted benzoimidazolen-1,2-yl. Non-limiting examples include the ring systems shown below.

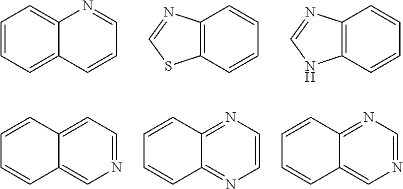

The rest of the molecule (e.g. O— or O═C— and —Y) may attach at any position on the ring system where a hydrogen would be present in the parent molecule. Similarly, a substitutent may be present at any position where a hydrogen atom would be present in the parent molecule.

In some embodiments, Ar may also be optionally substituted C₆₋₁₀ aryl, such as optionally substituted -phenyl- or optionally substituted -naphthyl-. In some embodiments, Ar may be fused bicyclic aryl or aryl.

Alternatively, in other embodiments, Ar may be optionally substituted heterocyclyl; or optionally substituted polycyclic moiety. In some embodiments, Ar may be optionally substituted isoindolinyl.

In some embodiments, each group described above may have 1, 2, 3, or 4 substituents independently selected from: C₁₋₁₀ alkyl such as CH₃ (e.g. methyl), C₂H₅ (e.g. ethyl), C₃H₇ (e.g. propyl isomers such as n-propyl, iso-propyl, etc.), C₄H₉ (e.g. butyl isomers), C₅H₁₁ (e.g. pentyl isomers), C₆H₁₃ (e.g. hexyl isomers), C₇H₁₅ (e.g. heptyl isomers), cyclopropyl, cyclobutyl, cyclopentyl, cyclohexyl, etc.; C₁₋₁₀ perfluoroalkyl such as CF₃ (e.g. trifluoromethyl), C₂F₅ (e.g. perfluoroethyl), C₃F₇ (e.g. perfluoropropyl isomers), C₄F₉ (e.g. perfluorobutyl isomers), C₅F₁₁ (e.g. perfluoropentyl isomers), C₆F₁₃ (e.g. perfluorohexyl isomers), C₇F₁₅ (e.g. perfluoroheptyl isomers), perfluorocyclopropyl, perfluorocyclobutyl, perfluorocyclopentyl, perfluorocyclohexyl, etc.; halo such as F, Cl, Br, I, etc; alkoxy such as —OCH₃, —OC₂H₅, —OC₃H₇, —OC₄H₉, —OC₅H₁₁, —OC₆H₁₃, —OC₇H₁₅, —O-cyclopropyl, —O-cyclobutyl, —O-cyclopentyl, —O-cyclohexyl, etc.; or C₁₋₁₀ perfluoralkoxy such as —OCF₃, —OC₂F₅, —OC₃F₇, —OC₄F₉, —OC₅F₁₁, —OC₆F₁₃, —OC₇F₁₅, —O-perfluorocyclopropyl, —O-perfluorocyclobutyl, —O-perfluorocyclopentyl, —O-perfluorocyclohexyl, etc. In some embodiments, unless otherwise specified, “optionally substituted” is defined as optional substitution with one or more group(s) individually and independently selected from C₁-C₆ alkyl, C₂-C₆ alkenyl, C₂-C₆ alkynyl, C₃-C₆ cycloalkyl, C₃-C₆ heterocycloalkyl, aryl, heteroaryl, halo, cyano, hydroxy, C₁-C₆ alkoxy, aryloxy, sulfhydryl, C₁-C₆ alkylthio, arylthio, mono- and di-(C₁-C₆)alkyl amino, quaternary ammonium salts, amino(C₁-C₆)alkoxy, hydroxy(C₁-C₆)alkylamino, amino(C₁-C₆)alkylthio, cyanoamino, nitro, carbamyl, oxo, carbonyl, carboxy, sulfamyl, sulfonyl, sulfinyl, thiocarbonyl, or thiocarboxy.

In some embodiments, Ar is selected from:

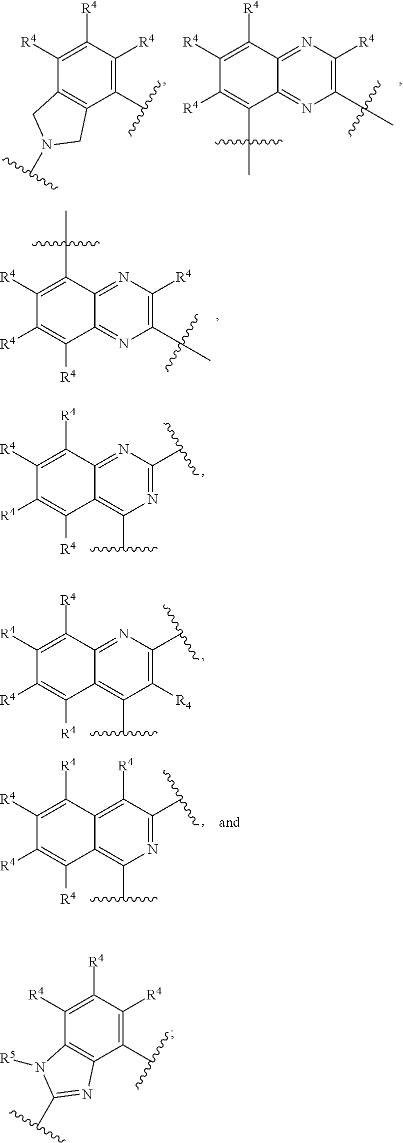

wherein each R⁴ is separately selected, where R⁴ is independently selected from the group consisting of H (hydrogen), halo, C₁₋₆alkyl optionally substituted with up to five fluoro, and C₁₋₆alkoxy optionally substituted with up to five fluoro; and R⁵ is selected from the group consisting of H (hydrogen), and C₁₋₆alkyl optionally substituted with up to five fluoro.

In some embodiments, Ar is optionally substituted benzoimidazolen-1,2-yl.

With respect to Formula I, z may be 0 or 1. Thus, some embodiments may be further represented by Formula I-1 or I-2.

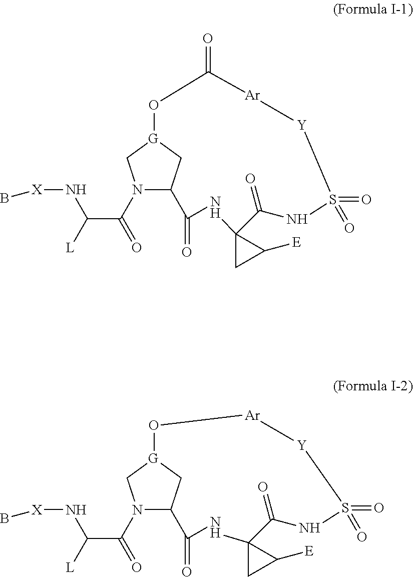

With respect to Formula I, Formula I-1, and Formula I-2, G may be

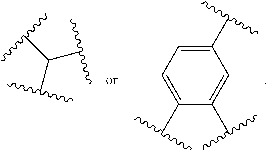

In some embodiments, G is

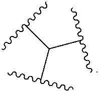

Thus, some embodiments may be further represented by Formula I-3.

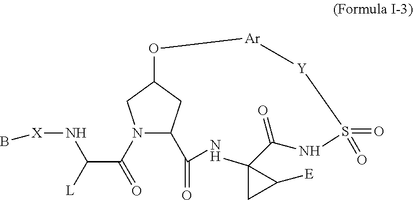

With respect to Formula I, Formula I-1, Formula I-2, and Formula I-3, X is a bond, C(═O), CO₂, CONH, SO₂, SO₃, or SO₂NH. In some embodiments, X is a bond, C(═O), CO₂, CONH, SO₂, or SO₂NH. In some embodiments, X is a bond, C(═O), CO₂, CONH, or SO₂. In other embodiments, X is a bond, CO, CO₂, or CONH. In yet other embodiments, X is a bond or CO₂. In some embodiments, the C (carbon) or S (sulfur) may attach to the adjacent nitrogen atom, and the O (oxygen) or N (nitrogen), if present, may attach to B, such that the B—X—NH— of one of the above formulas may represented by:

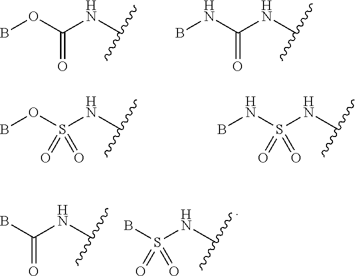

With respect to Formula I, Formula I-1, Formula I-2, and Formula I-3, B is H (hydrogen), optionally substituted C₆₋₁₀ aryl, optionally substituted C₂₋₁₀ heteroaryl, or optionally substituted C₁₋₁₀ hydrocarbyl. In some embodiments, B is H (hydrogen) or C₁₋₆ alkyl. In some embodiments, B is H (hydrogen) or t-butyl.

In some embodiments, B may be H (hydrogen), optionally substituted C₆₋₁₀ aryl such as optionally substituted phenyl or optionally substituted naphthyl; optionally substituted C₂₋₁₀ heteroaryl such as substituted benzooxazol-2-yl; optionally substituted benzothiazol-2-yl; optionally substituted benzoimidazol-2-yl; optionally substituted benzothiazol-2-yl; optionally substituted isoindolin-2-yl; or an optionally substituted pyridinyl, optionally substituted imidazolyl, optionally substituted thiazolyl, optionally substituted oxazolyl, optionally substituted thienyl, or optionally substituted furyl; or C₁₋₁₀ hydrocarbyl such as methyl, ethyl, ethenyl, propyl isomers (such as n-propyl, isopropyl, etc,), propenyl isomers, cyclopropyl, butyl isomers, butenyl isomers, cyclobutyl isomers (such as cyclobutyl, methylcyclopropyl, etc.), pentyl isomers, pentenyl isomers, cyclopentyl isomers, hexyl isomers, hexenyl isomers, cyclohexyl isomers, etc. In some embodiments, B may be C₁₋₆ alkyl.

With respect to Formula I, Formula I-1, Formula I-2, and Formula I-3, L is H (hydrogen) or optionally substituted C₁₋₁₀ hydrocarbyl. C₁₋₁₀ hydrocarbyl may be methyl, ethyl, ethenyl, propyl isomers (such as n-propyl, isopropyl, etc,), propenyl isomers, cyclopropyl, butyl isomers, butenyl isomers, cyclobutyl isomers (such as cyclobutyl, methylcyclopropyl, etc.), pentyl isomers, pentenyl isomers, cyclopentyl isomers, hexyl isomers, hexenyl isomers, cyclohexyl isomers, etc. In some embodiments, L may be C₁₋₆ alkyl, such as methyl, ethyl, propyl isomers, cyclopropyl, butyl isomers, cyclobutyl isomers, pentyl isomers, cyclopentyl isomers, hexyl isomers, cyclohexyl isomers, etc., or C₃₋₅ alkyl such as propyl isomers, cyclopropyl, butyl isomers, cyclobutyl isomers, pentyl isomers, cyclopentyl isomers, etc. In some embodiments, L is t-butyl.

With respect to Formula I, Formula I-1, Formula I-2, and Formula I-3, E is H (hydrogen) or optionally substituted C₁₋₆ hydrocarbyl. C₁₋₆ hydrocarbyl may be methyl, ethyl, ethenyl, propyl isomers (such as n-propyl, isopropyl, etc.), propenyl isomers, cyclopropyl, butyl isomers, butenyl isomers, cyclobutyl isomers (such as cyclobutyl, methylcyclopropyl, etc.), pentyl isomers, pentenyl isomers, cyclopentyl isomers, hexyl isomers, hexenyl isomers, or cyclohexyl isomers, etc. In some embodiments, E may be ethyl, vinyl, or cyclopropyl. In some embodiments, E may be C₁₋₆ alkyl, such as methyl, ethyl, propyl isomers, cyclopropyl, butyl isomers, cyclobutyl isomers, pentyl isomers, cyclopentyl isomers, hexyl isomers, cyclohexyl isomers, etc. In some embodiments, E is ethyl.

In some embodiments, X is a bond and B is H (hydrogen). In other embodiments, B is ethyl, n-propyl, isopropyl, n-butyl, isobutyl, sec-butyl, t-butyl, or a pentyl isomer; E is methyl, ethyl, propyl, or vinyl; and L is ethyl, n-propyl, isopropyl, n-butyl, isobutyl, sec-butyl, tert-butyl, or a pentyl isomer. In some embodiments, B is t-butyl, E is ethyl, and L is t-butyl.

With respect to Formula I, Formula I-1, Formula I-2, and Formula I-3, Y is (L₁)ₚ; wherein p is an integer from 5 to 12; each L₁ is separately selected, where L₁ is selected from the group consisting of C(R²)₂, NR³, O (oxygen), —(R²)C═C(R²)—, C(═O), C₃₋₇ cycloalkyl, optionally substituted aryl, optionally substituted polycyclic moiety, optionally substituted heterocycle and optionally substituted heteroaryl.

Each R² is separately selected, where R² is selected from the group consisting of H (hydrogen), C₁₋₆alkoxy, aryl, halo, hydroxy, R^{a}R^{b}N—,C₁₋₆alkyl optionally substituted with up to 5 halo, and C₁₋₆alkoxy optionally substituted with up to 5 halo, or optionally two vicinal R² and the carbons to which they are attached are together a fused three- to six-membered carbocyclic ring optionally substituted with up to two C₁₋₆alkyl groups, or optionally two geminal R² and the carbon to which they are attached are together a fused three- to six-membered carbocyclic ring optionally substituted with up to two C₁₋₆alkyl groups; and each R^{a}R^{b}N is separately selected, wherein R^{a} and R^{b} are each separately selected from the group consisting of hydrogen, C₂₋₆alkenyl, and C₁₋₆alkyl.

Each R³ is separately selected, where R³ is selected from the group consisting of hydrogen, optionally substituted aryl, optionally substituted heteroaryl, and optionally substituted C₁₋₆alkyl.

In some embodiments Y may be represented by:

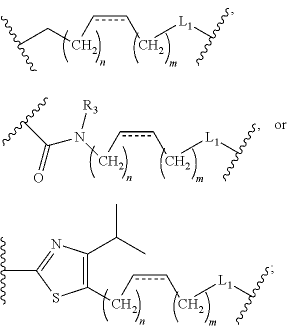

where the dashed line represents the presence or absence of a bond, and if present, the resulting double bond may be cis or trans; m and n are independently 0, 1, 2, 3, 4, 5, or 6. In some embodiments, the sum of m and n is 2, 3, 4, 5, 6, 7, 8, 9, or 10. In some embodiments, the sum of m and n is 4, 5, 6, or 7. In some embodiments, L₁ may be selected from the group consisting of C₃₋₇ cycloalkyl or C(R²)₂. In some embodiments, L₁ may be cyclopropyl or CH₂.

In some embodiments Y may be represented by:

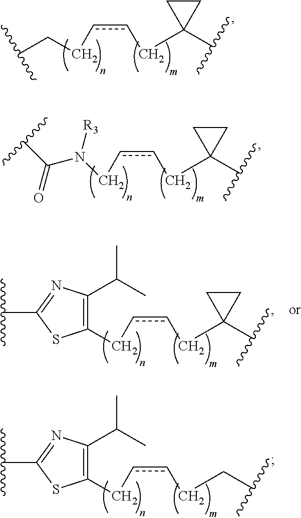

where the dashed line represents the presence or absence of a bond, and if present, the resulting double bond may be cis or trans; m and n are independently 0, 1, 2, 3, 4, 5, or 6. In some embodiments, the sum of m and n is 2, 3, 4, 5, 6, 7, 8, 9, or 10. In some embodiments, the sum of m and n is 4, 5, 6, or 7.

In some embodiments, the compound represented by Formula I has the structure of formula Ia, Ib, or Ic:

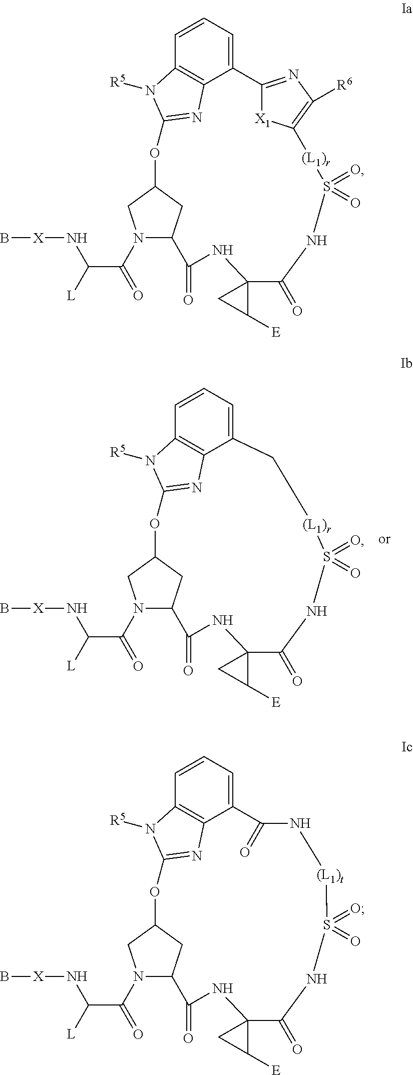

wherein r is an integer from 4 to 8; t is an integer from 3 to 7; R⁵ is selected from the group consisting of H (hydrogen), and C₁₋₆alkyl optionally substituted with up to five fluoro; R⁶ is selected from the group consisting of mono-(C₁-C₆)alkylamino, di-(C₁-C₆)alkylamino, C₁₋₆alkyl optionally substituted with up to five fluoro, optionally substituted arylalkyl, optionally substituted aryl, optionally substituted heteroaryl, optionally substituted heterocyclyl, and optionally substituted polycyclic moiety; and X₁ is NH, O (oxygen), or S (sulfur).

Some embodiments provide a compound having the structure of Formula II:

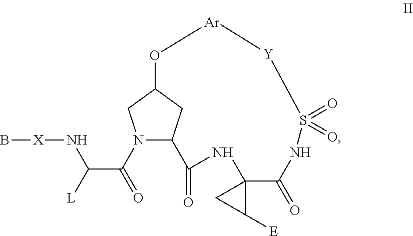

or a pharmaceutically acceptable salt thereof, wherein Ar is optionally substituted fused bicyclic heteroaryl, optionally substituted C_{6 or 10} aryl; or optionally substituted polycyclic moiety. In some embodiments, Ar can be an optionally substituted C₅₋₁₀ fused bicyclic heteroaryl, optionally substituted C_{6 or 10} aryl; or optionally substituted polycyclic moiety.

In some embodiments, Ar can be C₅₋₁₀ fused bicyclic heteroaryl, C_{6 or 10} aryl; or polycyclic moiety, each optionally substituted with one or more groups independently selected from the group consisting of halo, C₁₋₆alkyl optionally substituted with up to five fluoro, and C₁₋₆alkoxy optionally substituted with up to five fluoro.

In some embodiments, Ar is C₅₋₁₀ fused bicyclic heteroaryl, substituted with halo, C₁₋₆alkyl optionally substituted with up to five fluoro, or C₁₋₆alkoxy optionally substituted with up to five fluoro.

In some embodiments, Ar can be C₅₋₁₀ fused bicyclic heteroaryl, substituted with halo or C₁₋₆alkyl optionally substituted with up to five fluoro.

In some embodiments, Ar can be C₅₋₁₀ fused bicyclic heteroaryl, substituted with C₁₋₆alkoxy optionally substituted with up to five fluoro.

In some embodiments, Ar can be

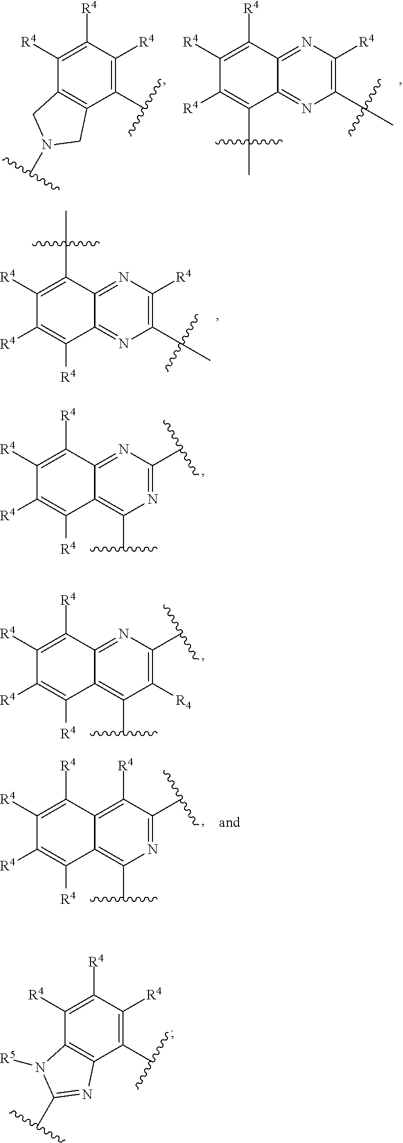

each R⁴ can separately be selected, where R⁴ can be independently selected from the group consisting of H (hydrogen), halo, and C₁₋₆alkyl optionally substituted with up to five fluoro, and C₁₋₆alkoxy optionally substituted with up to five fluoro; and R⁵ can be selected from the group consisting of H (hydrogen), and C₁₋₆alkyl optionally substituted with up to five fluoro.

In some embodiment, Ar is

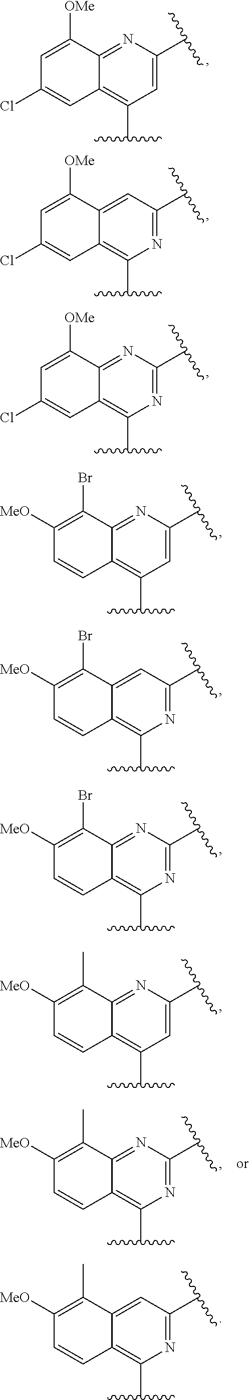

With respect to Formula II, Y is (L₁)ₚ; p is an integer from 5 to 9; each L₁ is separately selected, where L₁ is selected from the group consisting of C(R²)₂, NR³, O (oxygen), —(R²)C═C(R²)—, C(═O), C₃₋₇ cycloalkyl, optionally substituted aryl, optionally substituted polycyclic moiety, optionally substituted heterocycle and optionally substituted heteroaryl.

Each R² is separately selected, where R² is selected from the group consisting of H (hydrogen), C₁₋₆alkoxy, C₁₋₆alkyl, aryl, halo, hydroxy, R^{a}R^{b}N—,C₁₋₆alkyl optionally substituted with up to 5 halo, and C₁₋₆alkoxy optionally substituted with up to 5 halo, or optionally two vicinal R² and the carbons to which they are attached are together a fused three- to six-membered carbocyclic ring optionally substituted with up to two C₁₋₆alkyl groups, or optionally two geminal R² and the carbon to which they are attached are together a fused three- to six-membered carbocyclic ring optionally substituted with up to two C₁₋₆alkyl groups; and each R^{a}R^{b}N is separately selected, wherein R^{a} and R^{b} are each separately selected from the group consisting of hydrogen, C₂₋₆alkenyl, and C₁₋₆alkyl.

Each R³ is separately selected, where R³ is selected from the group consisting of hydrogen, optionally substituted aryl, optionally substituted heteroaryl, optionally substituted C₁₋₆alkyl, and C₁₋₆alkyl optionally substituted with up to 5 halo;

In some embodiments, Y can be selected from the group consisting of

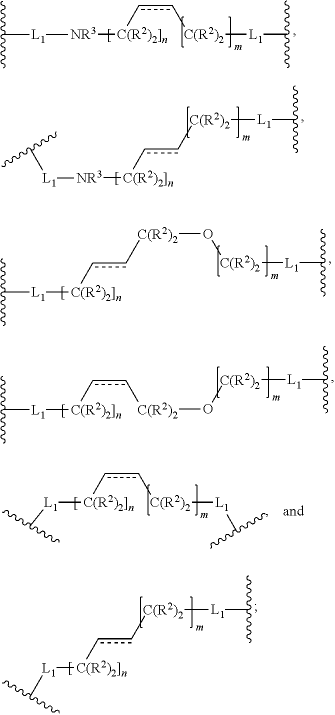

wherein m and n can each be independently 0, 1, 2, 3, 4, 5, or 6; each L₁ is separately selected from the group consisting of C(R²)₂, C(═O), C₃₋₇ cycloalkyl, and optionally substituted heteraryl; and the dashed line indicates an optional double bond. In some embodiments, the sum of m and n can be 4, 5, or 6. In some embodiments, the sum of m and n can be 2, 3, 4, 5, or 6.

In some embodiments, Y is selected from the group consisting of:

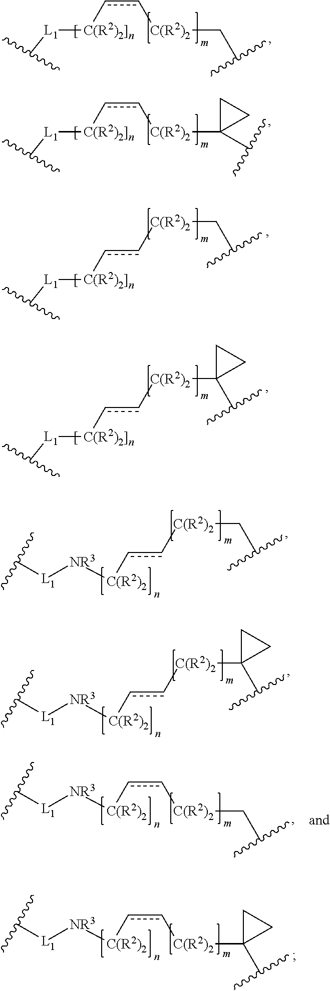

wherein m and n can each be independently 0, 1, 2, 3, 4, 5, or 6; each L₁ is separately selected from the group consisting of CH₂, C(═O), and

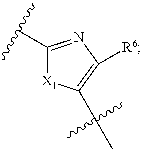

R⁶ is selected from the group consisting of mono-(C₁-C₆)alkylamino, di-(C₁-C₆)alkylamino, C₁₋₆alkyl optionally substituted with up to five fluoro, optionally substituted arylalkyl, optionally substituted aryl, optionally substituted heteroaryl, optionally substituted heterocyclyl, and optionally substituted polycyclic moiety; and X₁ is NH, O (oxygen), or S (sulfur). In some embodiments, the sum of m and n can be 4, 5, or 6. In some embodiments, the sum of m and n can be 2, 3, 4, 5, or 6.

In some embodiments, Y is selected from the group consisting of:

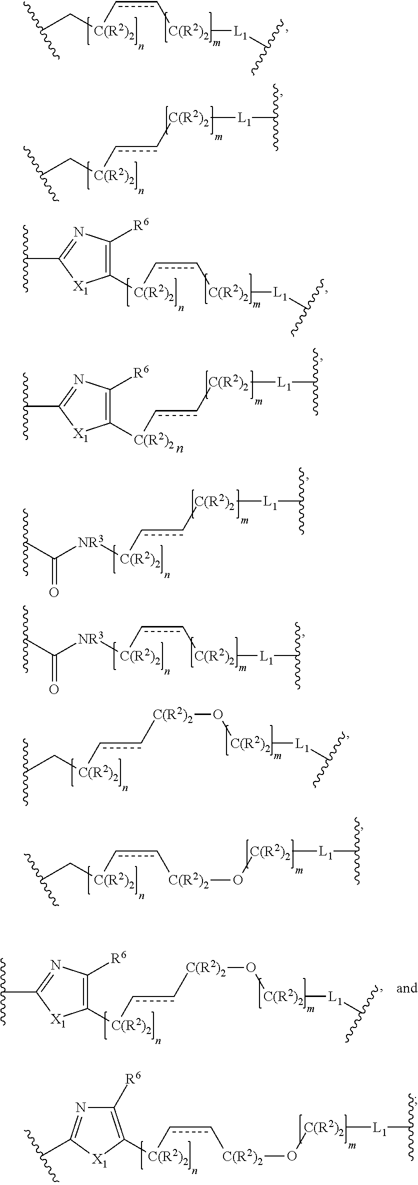

wherein m and n can each be independently 0, 1, 2, 3, 4, 5, or 6; R⁶ can be selected from the group consisting of H (hydrogen), halo, C₁₋₆alkyl optionally substituted with up to five fluoro, and C₁₋₆alkoxy optionally substituted with up to five fluoro; and X₁ can be NH, O (oxygen), or S (sulfur); X₁ can be NH, O (oxygen), or S (sulfur); and L₁ can be CH₂ or cyclopropyl.

In some embodiments, Y can be selected from the group consisting of

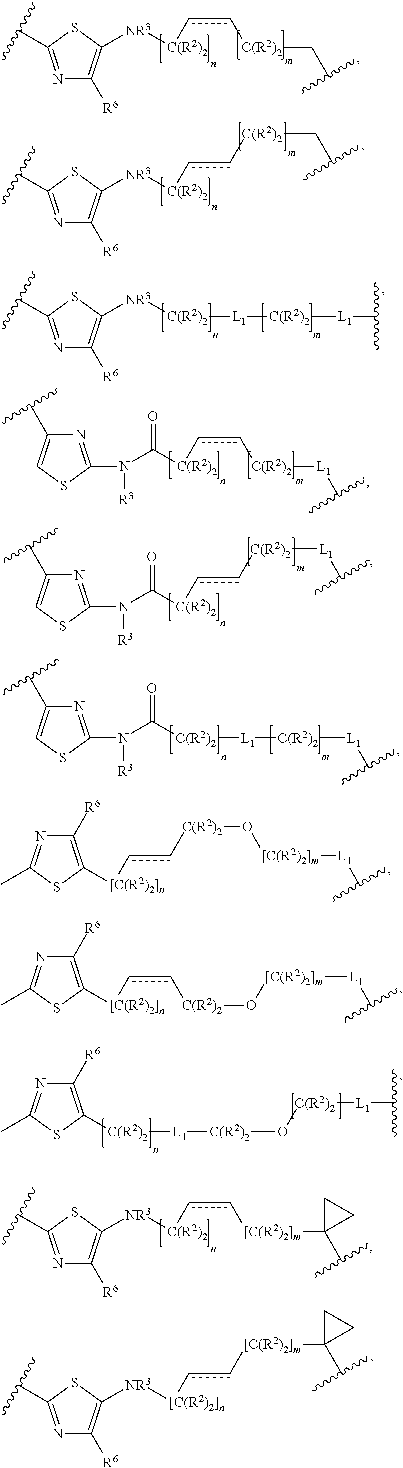

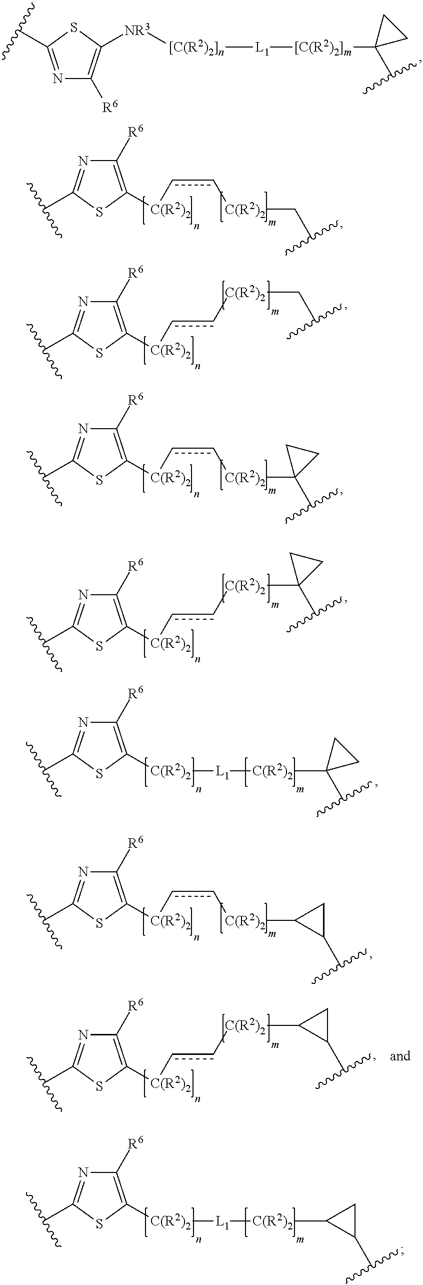

wherein m and n are each independently 0, 1, 2, 3, 4, 5, or 6; R⁶ can be selected from the group consisting of H (hydrogen), halo, C₁₋₆alkyl optionally substituted with up to five fluoro, and C₁₋₆alkoxy optionally substituted with up to five fluoro; and the dashed line indicates an optional double bond.

In some embodiments, Y can be selected from the group consisting of

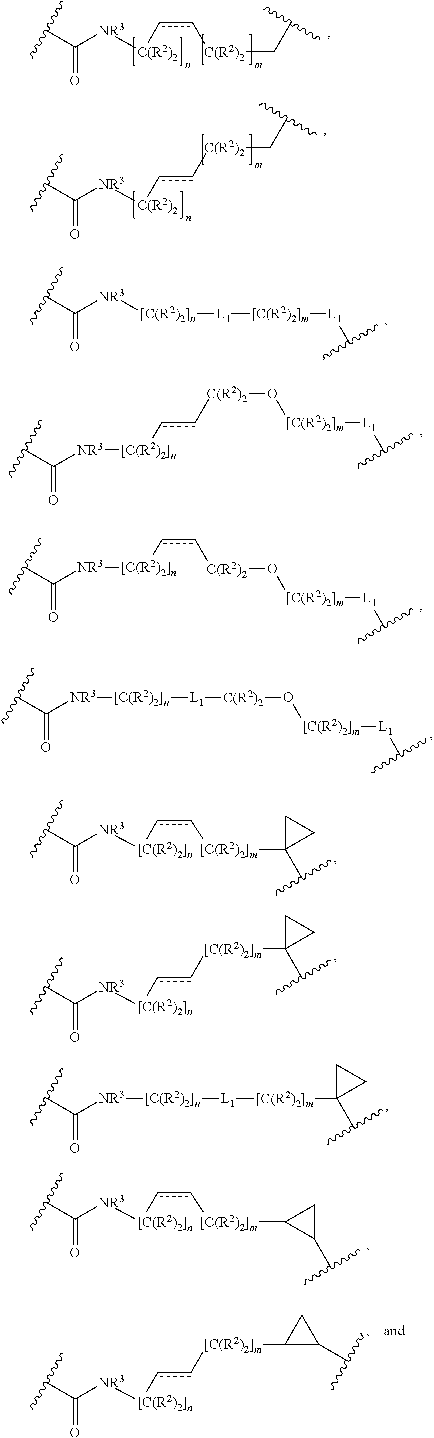

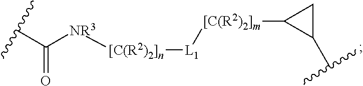

wherein m and n are each independently 0, 1, 2, 3, 4, 5, or 6; and R³ can be selected from the group consisting of H (hydrogen), and C₁₋₆alkyl optionally substituted with up to five fluoro; and the dashed line indicates an optional double bond.

In some embodiments, with respect to Formula II, X can be a bond, C(═O), CO₂, CONH, SO₂, SO₃, or SO₂NH. In some embodiments, X is a bond, C(═O), CO₂, CONH, or SO₂. In other embodiments, X can be a bond, C(═O), CO₂, or CONH. In some embodiments, X can be H or CO₂. In some embodiments, with respect to Formula II unless otherwise specified, “optionally substituted” is defined as optional substitution with one or more group(s) individually and independently selected from C₁-C₆ alkyl, C₂-C₆ alkenyl, C₂-C₆ alkynyl, C₃-C₆ cycloalkyl, C₃-C₆ heterocycloalkyl, aryl, heteroaryl, halo, cyano, hydroxy, C₁-C₆ alkoxy, aryloxy, sulfhydryl, C₁-C₆ alkylthio, arylthio, mono- and di-(C₁-C₆)alkyl amino, quaternary ammonium salts, amino(C₁-C₆)alkoxy, hydroxy(C₁-C₆)alkylamino, amino(C₁-C₆)alkylthio, cyanoamino, nitro, carbamyl, oxo, carbonyl, carboxy, sulfamyl, sulfonyl, sulfinyl, thiocarbonyl, or thiocarboxy.

With respect to Formula II, B can be H (hydrogen), optionally substituted C₁₋₆ alkyl, optionally substituted C₂₋₆ alkenyl, optionally substituted C₂₋₆ alkynyl, optionally substituted C₃₋₇ cycloalkyl, optionally substituted C_{6 or 10} aryl, optionally substituted heteroaryl, or optionally substituted heterocycle. In some embodiments, B can be optionally substituted C₅₋₁₀ heteroaryl, or optionally substituted C₅₋₁₀ heterocycle. In some embodiments, B can be H or C₁₋₆ alkyl. In some embodiments, B can be H (hydrogen) or t-butyl.

In some embodiments, B may be H (hydrogen), optionally substituted C₆₋₁₀ aryl such as optionally substituted phenyl or optionally substituted naphthyl; optionally substituted C₂₋₁₀ heteroaryl such as substituted benzooxazol-2-yl; optionally substituted benzothiazol-2-yl; optionally substituted benzoimidazol-2-yl; optionally substituted benzothiazol-2-yl; optionally substituted isoindolin-2-yl; or an optionally substituted pyridinyl, optionally substituted imidazolyl, optionally substituted thiazolyl, optionally substituted oxazolyl, optionally substituted thienyl, or optionally substituted furyl; or C₁₋₁₀ hydrocarbyl such as methyl, ethyl, ethenyl, propyl isomers (such as n-propyl, isopropyl, etc,), propenyl isomers, cyclopropyl, butyl isomers, butenyl isomers, cyclobutyl isomers (such as cyclobutyl, methylcyclopropyl, etc.), pentyl isomers, pentenyl isomers, cyclopentyl isomers, hexyl isomers, hexenyl isomers, cyclohexyl isomers, etc. In some embodiments, B may be C₁₋₆ alkyl.

In some embodiments, X can be a bond; and B can be H, or B can be C_{6 or 10} aryl, C₅₋₁₀ heteroaryl, or C₅₋₁₀ heterocycle, each optionally substituted with one or more groups independently selected from the group consisting of halo, hydroxy, C₁₋₆alkyl optionally substituted with up to five fluoro, and C₁₋₆alkoxy optionally substituted with up to five fluoro. In some embodiments, X can be —C(═O)O—; and B can be optionally substituted C₁₋₆ alkyl. In some embodiments, X is a bond and B is H (hydrogen). In some embodiments B—X—NH— can be:

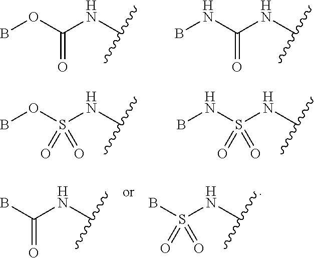

With respect to Formula II, Z is H (hydrogen) or optionally substituted C₁₋₁₀ hydrocarbyl. C₁₋₁₀ hydrocarbyl may be methyl, ethyl, ethenyl, propyl isomers (such as n-propyl, isopropyl, etc,), propenyl isomers, cyclopropyl, butyl isomers, butenyl isomers, cyclobutyl isomers (such as cyclobutyl, methylcyclopropyl, etc.), pentyl isomers, pentenyl isomers, cyclopentyl isomers, hexyl isomers, hexenyl isomers, cyclohexyl isomers, etc. In some embodiments, Z may be C₁₋₆ alkyl, such as methyl, ethyl, propyl isomers, cyclopropyl, butyl isomers, cyclobutyl isomers, pentyl isomers, cyclopentyl isomers, hexyl isomers, cyclohexyl isomers, etc., or C₃₋₅ alkyl such as propyl isomers, cyclopropyl, butyl isomers, cyclobutyl isomers, pentyl isomers, cyclopentyl isomers, etc. In some embodiments, L is t-butyl.

With respect to Formula II, E is H (hydrogen) or optionally substituted C₁₋₆ hydrocarbyl. C₁₋₆ hydrocarbyl may be methyl, ethyl, ethenyl, propyl isomers (such as n-propyl, isopropyl, etc.), propenyl isomers, cyclopropyl, butyl isomers, butenyl isomers, cyclobutyl isomers (such as cyclobutyl, methylcyclopropyl, etc.), pentyl isomers, pentenyl isomers, cyclopentyl isomers, hexyl isomers, hexenyl isomers, or cyclohexyl isomers, etc. In some embodiments, E may be ethyl, vinyl, or cyclopropyl. In some embodiments, E may be C₁₋₆ alkyl, such as methyl, ethyl, propyl isomers, cyclopropyl, butyl isomers, cyclobutyl isomers, pentyl isomers, cyclopentyl isomers, hexyl isomers, cyclohexyl isomers, etc. In some embodiments, E is ethyl.

In some embodiments, Z is C₁₋₆ alkyl; and E is C₁₋₆ alkyl or C₂₋₆ alkenyl. In some embodiments, Z can be optionally substituted C₁₋₆ alkyl; and E can be optionally substituted C₁₋₆ alkyl or optionally substituted C₂₋₆ alkenyl.

In some embodiments, the compound represented by Formula II is not selected from the group consisting of:

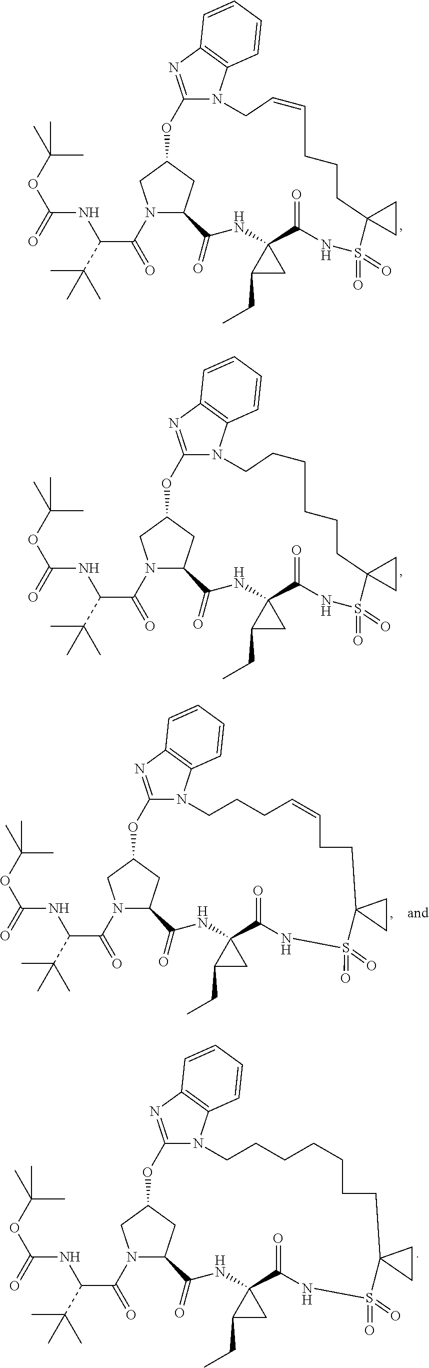

In some embodiments, the compound of Formula II may have the structure of Formula IIa:

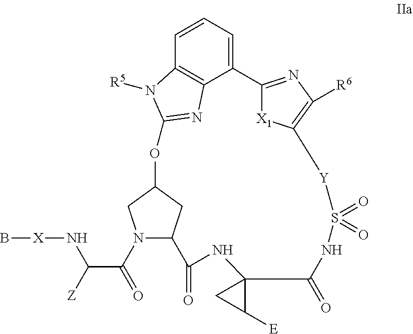

wherein Y is (L₁)ᵣ; r is an integer from 4 to 8; R⁵ is selected from the group consisting of H (hydrogen), and C₁₋₆alkyl optionally substituted with up to five fluoro; R⁶ is selected from the group consisting of mono-(C₁-C₆)alkylamino, di-(C₁-C₆)alkylamino, C₁₋₆alkyl optionally substituted with up to five fluoro, optionally substituted arylalkyl, optionally substituted aryl, optionally substituted heteroaryl, optionally substituted heterocyclyl, and polycyclic moiety; and X₁ is NH, O (oxygen), or S (sulfur).

### Compositions

The present embodiments further provide compositions, including pharmaceutical compositions, comprising compounds of the general Formulae I, Ia, Ib, Ic, II, and IIa or any compounds disclosed herein.

A subject pharmaceutical composition comprises a subject compound; and a pharmaceutically acceptable excipient. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The present embodiments provide for a method of inhibiting NS3/NS4 protease activity comprising contacting a NS3/NS4 protease with a compound disclosed herein.

The present embodiments provide for a method of treating hepatitis by modulating NS3/NS4 protease comprising contacting a NS3/NS4 protease with a compound disclosed herein.

Example compounds of Formula I, Ia, Ib, Ic, II, and IIa include compound number 101-113 as set forth herein.

Preferred embodiments provide a method of treating a hepatitis C virus infection in an individual, the method comprising administering to the individual an effective amount of a composition comprising a preferred compound.

Preferred embodiments provide a method of treating liver fibrosis in an individual, the method comprising administering to the individual an effective amount of a composition comprising a preferred compound.

Preferred embodiments provide a method of increasing liver function in an individual having a hepatitis C virus infection, the method comprising administering to the individual an effective amount of a composition comprising a preferred compound.

In some embodiments, a subject compound may inhibit the enzymatic activity of a hepatitis virus C(HCV) NS3 protease. Whether a subject compound inhibits HCV NS3 protease can be readily determined using any known method. Typical methods may involve a determination of whether an HCV polyprotein or other polypeptide comprising an NS3 recognition site is cleaved by NS3 in the presence of the agent. In many embodiments, a subject compound inhibits NS3 enzymatic activity by a detectable amount, such as at least about 10%, at least about 15%, at least about 20%, at least about 25%, at least about 30%, at least about 40%, at least about 50%, at least about 60%, at least about 70%, at least about 80%, or at least about 90%, or more, compared to the enzymatic activity of NS3 in the absence of the compound.

In many embodiments, a subject compound may inhibit enzymatic activity of an HCV NS3 protease with an IC₅₀ of less than about 50 μM, e.g., a subject compound inhibits an HCV NS3 protease with an IC₅₀ of less than about 40 μM, less than about 25 μM, less than about 10 μM, less than about 1 μM, less than about 100 nM, less than about 80 nM, less than about 60 nM, less than about 50 nM, less than about 25 nM, less than about 10 nM, or less than about 1 nM, or less.

In many embodiments, a subject compound may inhibit the enzymatic activity of a hepatitis virus C(HCV) NS3 helicase. Whether a subject compound inhibits HCV NS3 helicase can be readily determined using any known method. In many embodiments, a subject compound inhibits NS3 enzymatic activity by a detectable amount, such as at least about 10%, at least about 15%, at least about 20%, at least about 25%, at least about 30%, at least about 40%, at least about 50%, at least about 60%, at least about 70%, at least about 80%, or at least about 90%, or more, compared to the enzymatic activity of NS3 in the absence of the compound.

In many embodiments, a subject compound may inhibit HCV viral replication. For example, a subject compound may inhibit HCV viral replication by a detectable amount, such as at least about 10%, at least about 15%, at least about 20%, at least about 25%, at least about 30%, at least about 40%, at least about 50%, at least about 60%, at least about 70%, at least about 80%, or at least about 90%, or more, compared to HCV viral replication in the absence of the compound. Whether a subject compound inhibits HCV viral replication can be determined using methods known in the art, including an in vitro viral replication assay.

### Treating a Hepatitis Virus Infection

The methods and compositions described herein may be generally useful in treatment of an of HCV infection.

Whether a subject method is effective in treating an HCV infection may be determined by a reduction in viral load, a reduction in time to seroconversion (virus undetectable in patient serum), an increase in the rate of sustained viral response to therapy, a reduction of morbidity or mortality in clinical outcomes, or other indicator of disease response.

In general, an effective amount of a compound disclosed herein, and optionally one or more additional antiviral agents, is an amount that is effective to produce a detectable effect described herein, such as to reduce viral load or achieve a sustained viral response to therapy.

Whether a subject method is effective in treating an HCV infection may be determined by directly or indirectly observing or measuring any effect or parameter which may be associated with the effective treatment of HCV infection such as, but not limited to, measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

Some embodiments involve administering an effective amount of a compound disclosed herein optionally in combination with an effective amount of one or more additional antiviral agents. In some embodiments, an effective amount of a compound disclosed herein, and optionally one or more additional antiviral agents, may be an amount that is effective to reduce viral titers to undetectable levels, e.g., to about 1000 to about 5000, to about 500 to about 1000, or to about 100 to about 500 genome copies/mL serum. In some embodiments, an effective amount of a compound disclosed herein, and optionally one or more additional antiviral agents, may be an amount that is effective to reduce viral load to lower than 100 genome copies/mL serum.

In some embodiments, an effective amount of a compound disclosed herein, and optionally one or more additional antiviral agents, may be an amount that is effective to achieve a 1.5-log, a 2-log, a 2.5-log, a 3-log, a 3.5-log, a 4-log, a 4.5-log, or a S-log reduction in viral titer in the serum of the individual.

In many embodiments, an effective amount of a compound disclosed herein, and optionally one or more additional antiviral agents, may be an amount that is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a compound disclosed herein, and optionally one or more additional antiviral agents, is an amount effective to reduce ALT levels to less than about 45 IU/mL serum.

A therapeutically effective amount of a compound disclosed herein, and optionally one or more additional antiviral agents, may be an amount that is effective to reduce a serum level of a marker of liver fibrosis by a detectable amount, such as at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

In many embodiments, an effective amount of a compound disclosed herein and an additional antiviral agent may be a synergistic amount. As used herein, a “synergistic combination” or a “synergistic amount” of a compound disclosed herein and an additional antiviral agent is a combined dosage that is more effective in the therapeutic or prophylactic treatment of an HCV infection than the incremental improvement in treatment outcome that could be predicted or expected from a merely additive combination of (i) the therapeutic or prophylactic benefit of a compound disclosed herein when administered at that same dosage as a monotherapy and (ii) the therapeutic or prophylactic benefit of the additional antiviral agent when administered at the same dosage as a monotherapy.

### Fibrosis

Some embodiments provide methods for treating liver fibrosis (including forms of liver fibrosis resulting from, or associated with, HCV infection), generally involving administering a therapeutic amount of a compound disclosed herein, and optionally one or more additional antiviral agents. Effective amounts of compounds disclosed herein, with and without one or more additional antiviral agents, as well as dosing regimens, are as discussed below.

Whether treatment with a compound disclosed herein, and optionally one or more additional antiviral agents, is effective in reducing liver fibrosis may be determined by any of a number of well-established techniques for measuring liver fibrosis and liver function. Liver fibrosis reduction may be determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score may be assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of a compound disclosed herein, and optionally one or more additional antiviral agents, may be an amount that effects a change of one unit or more in the fibrosis stage based on pre- and post-therapy liver biopsies. In particular embodiments, a therapeutically effective amount of a compound disclosed herein, and optionally one or more additional antiviral agents, reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment with a compound disclosed herein. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

An effective amount of a compound disclosed herein, and optionally one or more additional antiviral agents, may be an amount that is effective to increase an index of liver function by a detectable amount, such as at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of a compound disclosed herein, and optionally one or more additional antiviral agents, may be an amount that is effective to reduce a serum level of a marker of liver fibrosis by a detectable amount, such as at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of a compound disclosed herein, and optionally one or more additional antiviral agents, may be an amount that is effective in reducing the incidence (e.g., the likelihood that an individual will develop) of a disorder associated with cirrhosis of the liver by a detectable amount, such as at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with a compound disclosed herein, and optionally one or more additional antiviral agents, is effective in reducing the incidence of a disorder associated with cirrhosis of the liver can readily be determined by those skilled in the art.

Reduction in liver fibrosis increases liver function. Thus, the embodiments provide methods for increasing liver function, generally involving administering a therapeutically effective amount of a compound disclosed herein, and optionally one or more additional antiviral agents. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal level of alanine transaminase is about 45 IU per milliliter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of a compound disclosed herein, and optionally one or more additional antiviral agents, may be an amount that is effective to increase liver function by a detectable amount, such as at least about 10%, at least about 20%, at least about 30%, at least about 40%, at least about 50%, at least about 60%, at least about 70%, at least about 80%, or more. For example, a therapeutically effective amount of a compound disclosed herein, and optionally one or more additional antiviral agents, is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of a compound disclosed herein, and optionally one or more additional antiviral agents, is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

### Dosages, Formulations, and Routes of Administration

In the subject methods, the active agent(s) (e.g., compounds as described herein, and optionally one or more additional antiviral agents) may be administered to the host using any convenient means capable of resulting in the desired therapeutic effect. Thus, the agent may be incorporated into a variety of formulations for therapeutic administration. More particularly, the agents of the embodiments can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

### Formulations

The above-discussed active agent(s) may be formulated using well-known reagents and methods. Compositions are provided in formulation with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an agent may be formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents may include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

As such, administration of the agents can be achieved in various ways, including oral, buccal, rectal, parenteral, intraperitoneal, intradermal, subcutaneous, intramuscular, transdermal, intratracheal, etc., administration. In many embodiments, administration is by bolus injection, e.g., subcutaneous bolus injection, intramuscular bolus injection, and the like.

The pharmaceutical compositions of the embodiments can be administered orally, parenterally or via an implanted reservoir. Oral administration or administration by injection is preferred.

Subcutaneous administration of a pharmaceutical composition of the embodiments may be accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a pharmaceutical composition of the embodiments to a patient through the port is referred to herein as “a subcutaneous injection port delivery system.” In many embodiments, subcutaneous administration may be achieved by bolus delivery by needle and syringe.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents may be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents may be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents may be made into suppositories by mixing with a variety of bases such as emulsifying bases or water-soluble bases. The compounds of the embodiments can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the embodiments calculated in an amount sufficient to produce the desired effect in association with a pharmaceutically acceptable diluent, carrier or vehicle. The specifications for the novel unit dosage forms of the embodiments depend on the particular compound employed and the effect to be achieved, and the pharmacodynamics associated with each compound in the host.

The pharmaceutically acceptable excipients, such as vehicles, adjuvants, carriers or diluents, may be readily available to the public. Moreover, pharmaceutically acceptable auxiliary substances, such as pH adjusting and buffering agents, tonicity adjusting agents, stabilizers, wetting agents and the like, may be readily available to the public.

### Other Antiviral or Antifibrotic Agents

As discussed above, a subject method may in some embodiments be carried out by administering a compound disclosed herein, and optionally one or more additional antiviral agent(s).

In some embodiments, the method further may include administration of one or more interferon receptor agonist(s).

In other embodiments, the method may further include administration of pirfenidone or a pirfenidone analog.

Additional antiviral agents that are suitable for use in combination therapy may include, but are not limited to, nucleotide and nucleoside analogs. Non-limiting examples include azidothymidine (AZT) (zidovudine), and analogs and derivatives thereof; 2′,3′-dideoxyinosine (DDI) (didanosine), and analogs and derivatives thereof; 2′,3′-dideoxycytidine (DDC) (dideoxycytidine), and analogs and derivatives thereof; 2′,3′-didehydro-2′,3′-dideoxythymidine (D4T) (stavudine), and analogs and derivatives thereof; combivir; abacavir; adefovir dipoxil; cidofovir; ribavirin; ribavirin analogs; and the like.

In some embodiments, the method may further include administration of ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. Some embodiments also involve use of derivatives of ribavirin (see, e.g., U.S. Pat. No. 6,277,830). The ribavirin may be administered orally in capsule or tablet form, or in the same or different administration form and in the same or different route as the NS-3 inhibitor compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, the method may further includes administration of ritonavir. Ritonavir, 10-hydroxy-2-methyl-5-(1-methylethyl)-1-[2-(1-methylethyl)-4-thiazolyl]-3,6-dioxo-8,11-bis(phenylmethyl)-2,4,7,12-tetraazamidecan-13-oic acid, 5-thiazolylmethyl ester [5S-(5R*,8R*, 10R*,11R*)], available from Abbott Laboratories, is an inhibitor of the protease of the human immunodeficiency virus and also of the cytochrome P450 3A and P450 2D6 liver enzymes frequently involved in hepatic metabolism of therapeutic molecules in man.

In some embodiments, the method further includes administration of a protease inhibitor. In some embodiments, the method further includes administration of another NS5A inhibitor. In some embodiments, the method further includes administration of a helicase inhibitor. In some embodiments, the method further includes administration of a polymerase inhibitor.

In some embodiments, an additional antiviral agent may be administered during the entire course of NS3 inhibitor compound treatment. In other embodiments, an additional antiviral agent may be administered for a period of time that is overlapping with that of the NS3 inhibitor compound treatment, e.g., the additional antiviral agent treatment can begin before the NS3 inhibitor compound treatment begins and end before the NS3 inhibitor compound treatment ends; the additional antiviral agent treatment can begin after the NS3 inhibitor compound treatment begins and end after the NS3 inhibitor compound treatment ends; the additional antiviral agent treatment can begin after the NS3 inhibitor compound treatment begins and end before the NS3 inhibitor compound treatment ends; or the additional antiviral agent treatment can begin before the NS3 inhibitor compound treatment begins and end after the NS3 inhibitor compound treatment ends.

### Methods of Treatment

### Monotherapies

The compounds described herein may be used in acute or chronic therapy for HCV disease. In many embodiments, the compound may be administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The NS3 inhibitor compound can be administered 5 times per day, 4 times per day, tid, bid, qd, qod, biw, tiw, qw, qow, three times per month, or once monthly. In other embodiments, the NS3 inhibitor compound may be administered as a continuous infusion.

In many embodiments, a compound described herein may be administered orally.

In connection with the above-described methods for the treatment of HCV disease in a patient, an NS3 inhibitor compound as described herein may be administered to the patient at a dosage from about 0.01 mg to about 100 mg/kg patient bodyweight per day, in 1 to 5 divided doses per day. In some embodiments, the NS3 inhibitor compound may be administered at a dosage of about 0.5 mg to about 75 mg/kg patient bodyweight per day, in 1 to 5 divided doses per day.

The amount of active ingredient that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific NS3 inhibitor compound, the severity of the symptoms and the susceptibility of the subject to side effects. Preferred dosages for a given NS3 inhibitor compound may be readily determinable by those of skill in the art by a variety of means. A preferred means may be to measure the physiological potency of a given interferon receptor agonist.

In many embodiments, multiple doses of NS3 inhibitor compound are administered. For example, an NS3 inhibitor compound may be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies with a TNF-α Antagonist and an Interferon

Some embodiments provide a method of treating an HCV infection in an individual having an HCV infection, the method comprising administering an effective amount of an NS3 inhibitor, and effective amount of a TNF-α antagonist, and an effective amount of one or more interferons.

### Subjects Suitable for Treatment

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient.

Any of the above treatment regimens can be administered to individuals who have been diagnosed with an HCV infection. Any of the above treatment regimens can be administered to individuals having advanced or severe stage liver fibrosis as measured by a Knodell score of 3 or 4 or no or early stage liver fibrosis as measured by a Knodell score of 0, 1, or 2. Any of the above treatment regimens can be administered to individuals who have failed previous treatment for HCV infection (“treatment failure patients,” including non-responders and relapsers).

Individuals who have been clinically diagnosed as infected with HCV are of particular interest in many embodiments. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (RIBA). Such individuals may also, but need not, have elevated serum ALT levels.

Individuals who are clinically diagnosed as infected with HCV include naïve individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods described herein. In other embodiments, individuals suitable for treatment with the methods of the embodiments are patients with decompensated cirrhosis with clinical manifestations, including patients with far-advanced liver cirrhosis, including those awaiting liver transplantation. In still other embodiments, individuals suitable for treatment with the methods described herein include patients with milder degrees of fibrosis including those with early fibrosis (stages 1 and 2 in the METAVIR, Ludwig, and Scheuer scoring systems; or stages 1, 2, or 3 in the Ishak scoring system).

### Preparation of Compounds

### Methodology

The HCV protease inhibitors in the following sections can be prepared according to the procedures and schemes shown in each section. The numberings in each of the following Preparation of NS3 Inhibitor sections are meant for that specific section only, and should not be construed or confused with the same numberings in other sections.

### Section I

### Example 1

### 1-1. (1R,2R)-ethyl 1-amino-2-ethylcyclopropanecarboxylate, HCl salt

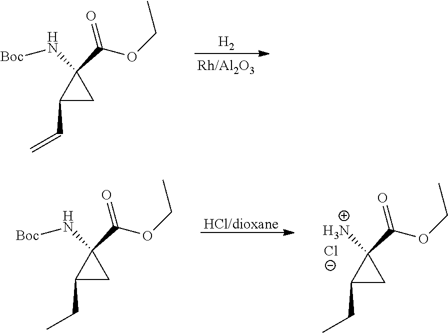

A solution of (1R,2S)-ethyl 1-(tert-butoxycarbonylamino)-2-vinylcyclopropane carboxylate (10 g) in ethyl acetate (150 mL) was hydrogenated at 35 psi in the presence of Rh/Al₂O₃ (500 mg) for two hours. The catalyst was filtered off and the solvent was evaporated under reduced pressure to give an oil (˜10 g) which was used on the next step without further purification. This product was dissolved in ethyl acetate (30 mL), 4N HCl-dioxane (29 mL, 117 mmol) was added, and the reaction was left overnight. The solvent was removed to give the title compound as a pale yellow solid. Yield: 7.5 g, ˜100%. ¹H-NMR (DMSO-d⁶), δ: 8.87 (br. s, 3H), 4.23 (m, 2H), 1.70-1.79 (m, 1H), 1.58-1.62 (m, 2H), 1.38-1.43 (m, 1H), 1.31 (dd, 1H), 1.22 (t, 3H), 0.90 (t, 3H).

### 1-2. 1-(pent-4-enyl)cyclopropane-1-sulfonamide

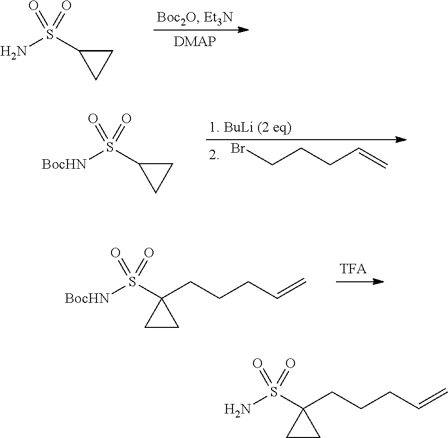

To a solution of cyclopropanesulfonamide (6 g, 50 mmol) in DCM (50 mL) were added triethylamine (7.5 mL, 74 mmol) followed by DMAP (0.3 g, 7.5 mmol) and di-tert-butyl dicarbonate (13 g, 59 mL) and the reaction was left stirring overnight. The solvent was removed; water, 2N HCl (40 mL) and ethyl acetate were added. The organic layer was washed with brine, dried over magnesium sulfate and evaporated to give Boc-cyclopropanesulfonamide as a white solid, used on the next step without further purification. Yield 9.73 g (88%).

To a solution of this intermediate (2.21 g, 10 mmol) in anhydrous THF (25 mL) was added BuLi (1.6 M solution in hexanes, 14.4 mL, 23 mmol) while stirring at −78° C. The reaction was stirred for one hour and 1-bromo-5-pentene was added at the same temperature. After further stirring for one hour the cooling bath was removed and the reaction was stirred overnight at room temperature. The reaction was quenched with water, acidified with 2N HCl (7 mL) and extracted with ethyl acetate. The organic phase was washed with brine, dried over magnesium sulfate and evaporated. The crude Boc intermediate was dissolved in DCM (5 mL) and trifluoroacetic acid (TFA) (5 mL). After one hour the reaction was evaporated under reduced pressure and co-evaporated twice with toluene. The residue was purified by column chromatography (25-35% ethyl acetate-hexane) to afford the target compound as pale yellow solid. Yield 1.55 g (82%). ¹H-NMR (CDCl₃), δ: 5.73-5.83 (m, 1H), 4.96-5.05 (m, 2H), 4.75 (br. s, 2H), 2.04-2.11 (m, 2H), 1.87-1.92 (m, 2H), 1.55-1.63 (m, 2H), 1.36-1.40 (m, 2H), 0.85-0.88 (m, 2H).

### Section II

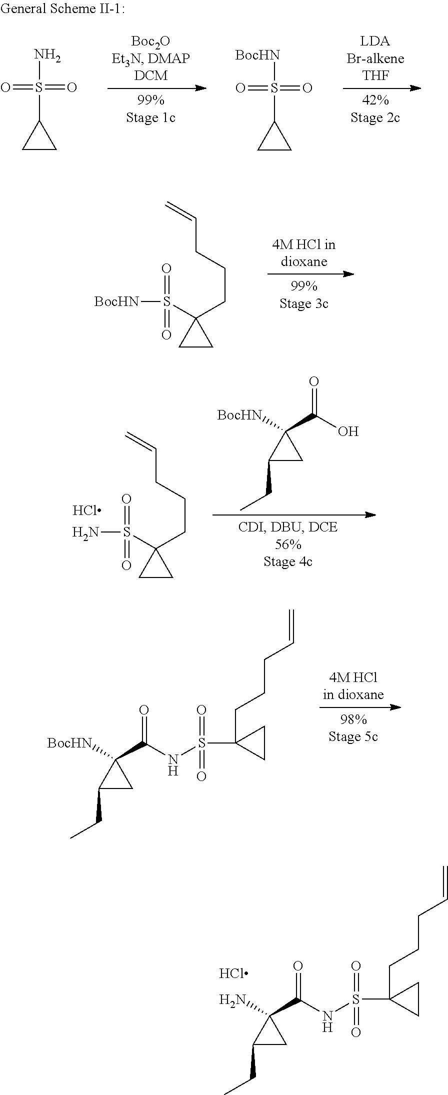

### Example II-1

### Stage 1c:

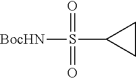

Cyclopropanesulfonamide (5.0 g, 41.26 mmol, 1.0 eq.), triethylamine (8.62 g, 61.90 mmol, 1.5 eq.) and dichloromethane (50 mL) were charged in a 100 mL round bottom flask. Di-tert-butyldicarbonate (10.8 g, 49.52 mmol, 1.2 eq.) and N,N-dimethylaminopyridine (252 mg, 2.06 mmol, 0.05 eq.) were added portion wise and the reaction mixture stirred at ambient temperature for 48 hours. The solvent was removed in vacuo and the residue diluted with water (20 mL). The aqueous phase was acidified to pH=1 with 1M hydrochloric acid, and extracted with ethyl acetate (3×100 mL). The organic extracts were combined, washed with water (100 mL) and brine (100 mL), dried over magnesium sulfate, filtered and the solvent removed in vacuo to give 9.1 g (99% yield) of the title compound as a white solid. ¹H NMR (500 MHz, CDCl₃) δ ppm 7.14 (br. s., 1H) 2.90 (tt, J=8.09, 4.73 Hz, 1H) 1.50-1.54 (m, 9H) 1.35-1.40 (m, 2H) 1.09-1.15 (m, 2H). LC-MS: purity 100% (UV), t_{R} 1.62 min m/z [M+Na]⁺243.95 (MET/CR/1278).

### Stage 2c:

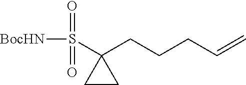

Stage 1c intermediate (2.0 g, 9.04 mmol, 1.0 eq.) and tetrahydrofuran (10 mL) were charged in a 25 mL round bottom flask and the reaction mixture cooled down to −78° C. Lithium diisopropylamide (1.8 M, 15 mL, 27.11 mmol, 3.0 eq.) was added dropwise and stirring continued for 1 hour. 5-Bromo-pent-1-ene (1.3 mL, 10.84 mmol, 1.22 eq.) was added dropwise and then the reaction mixture was left to warm up to ambient temperature and stirring was continued for another 4 hours. The reaction mixture was quenched with saturated aqueous ammonium chloride solution (40 mL) and extracted with ethyl acetate (3×50 mL). The organic extracts were combined, washed with brine (50 mL), dried over magnesium sulfate, filtered and the solvent removed in vacuo. The residue was purified by flash column chromatography using a ethyl acetate/heptanes gradient to give 1.12 g (42% yield) of the title compound as a beige solid. ¹H NMR (500 MHz, CDCl₃) δ ppm 6.82 (br. s., 1H) 5.65-5.89 (m, 1H) 5.04-5.10 (m, 1H) 4.91-5.04 (m, 1H) 2.01-2.15 (m, 2H) 1.81-1.93 (m, 2H) 1.62-1.69 (m, 2H) 1.55-1.60 (m, 2H) 1.51 (s, 9H) 0.95 (d, J=1.68 Hz, 2H). LC-MS: purity 100% (ELS), t_{R} 2.17 min m/z [M+Na]⁺312.00 (MET/CR/1278).

### Stage 3c:

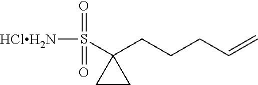

Stage 2c intermediate (1.12 g, 3.87 mmol, 1.0 eq.) and dioxane (5 mL) were charged into a 25 mL round bottom flask. 4M hydrogen chloride in dioxane (5 mL) was added dropwise and the reaction mixture stirred at 40° C. for 2 hours. The solvent was removed in vacuo to give 720 mg (99% yield, hydrochloride salt) of the title compound as a red oil. ¹H NMR (250 MHz, CDCl₃) δ ppm 5.63-5.96 (m, 1H) 4.87-5.17 (m, 2H) 4.43 (br. s., 2H) 2.09 (q, J=6.90 Hz, 2H) 1.79-2.01 (m, 2H) 1.49-1.72 (m, 2H) 1.30-1.48 (m, 2H) 0.80-0.96 (m, 2H). LC-MS: purity 100% (ELS), t_{R} 1.59 min m/z [M+H]⁺189.85 weak (MET/CR/1278).

### Stage 4c:

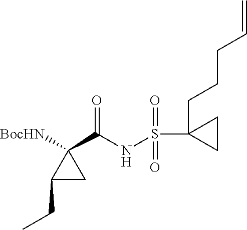

N-Boc-carboxylic acid (739 mg, 3.22 mmol, 1.0 eq.) and dichloroethane (8 mL) were charged in a 25 mL round bottom flask. 1,1′-carbonyldiimidazole (732 mg, 4.51 mmol, 1.4 eq.) was added as a single portion and the reaction mixture stirred at 50° C. for 2 hours. Stage 3c intermediate (732 mg, 3.87 mmol, 1.2 eq.), DBU (1.23 mL, 8.08 mmol, 2.5 eq.) and dichloroethane (2 mL) were mixed together and the resulting solution added dropwise to the reaction mixture at 50° C. Heating was continued for a further 15 hours. The reaction mixture was left to cool down to ambient temperature and aqueous citric acid (0.05 M, 50 mL) was added. The aqueous phase was further extracted with dichloromethane (3×50 mL). The organic extracts were combined, washed with water (50 mL) and brine (50 mL), dried over magnesium sulfate, filtered and the solvent removed in vacuo. The residue was purified by flash column chromatography using a ethyl acetate/heptanes gradient to give 720 mg (56% yield) of the title compound as a white solid. ¹H NMR (500 MHz, CDCl₃)δ ppm 9.59 (br. s., 1H) 5.69-5.85 (m, 1H) 5.14 (br. s., 1H) 4.99-5.05 (m, 1H) 4.94-4.99 (m, 1H) 2.06 (q, J=7.22 Hz, 2H) 1.83-1.91 (m, 2H) 1.65-1.73 (m, 2H) 1.56 (s, 3H) 1.53-1.56 (m, 1H) 1.49 (s, 9H) 1.45 (s, 2H) 1.06-1.13 (m, 1H) 1.02 (t, J=7.25 Hz, 3H) 0.88-0.99 (m, 2H). LC-MS: purity 100% (ELS), t_{R} 2.29 min m/z [M+Na]⁺ 423.05 (MET/CR/1278).

### Stage 5c:

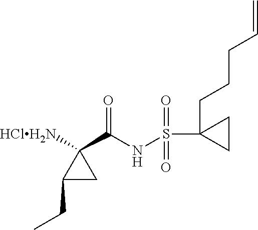

Stage 4c intermediate (720 mg, 1.80 mmol, 1.0 eq.) and dioxane (3 mL) were charged into a 25 mL round bottom flask. 4M hydrogen chloride in dioxane (4 mL) was added dropwise and the reaction mixture stirred at 40° C. for 2 hours. The solvent was removed in vacuo to give 590 mg (98% yield, hydrochloride salt) of the title compound as a beige solid. ¹H NMR (250 MHz, CDCl₃) δ ppm 5.63-5.96 (m, 1H) 4.87-5.17 (m, 2H) 4.43 (br. s., 2H) 2.09 (q, J=6.90 Hz, 2H) 1.79-2.01 (m, 2H) 1.49-1.72 (m, 2H) 1.30-1.48 (m, 2H) 0.80-0.96 (m, 2H). LC-MS: purity 100% (UV), t_{R} 1.49 min m/z [M+H]⁺ 301.05 (MET/CR/1278).

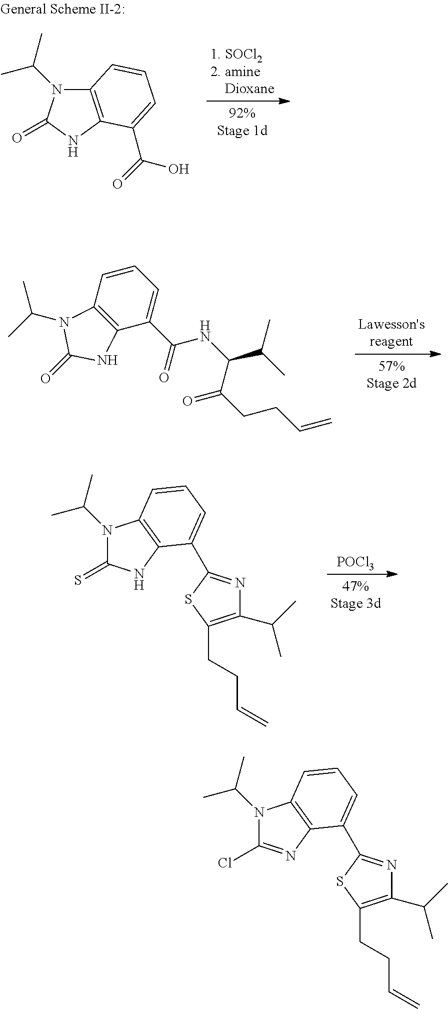

### Example II-2

### Stage 1d:

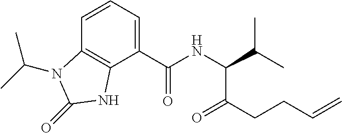

1-Isopropyl-2-oxo-4-carboxyl-benzimidazole (400 mg, 2.16 mmol, 1.0 eq.) and thionyl chloride (8 mL) were charged into a 25 mL round bottom flask and the reaction mixture stirred at ambient temperature for 2 hours. The excess thionyl chloride was then removed in vacuo and the residue diluted with dry dioxane (6 mL). Diisopropylethylamine (950 mg, 5.44 mmol, 3.0 eq.) was added dropwise over 5 min. The aminoketone hydrochloride salt (310 mg, 1.99 mmol, 1.1 eq.) previously dissolved in dioxane (3 mL) was added portionwise and the reaction mixture stirred at ambient temperature for 4 hours. The reaction mixture was quenched with water (50 mL) and extracted with ethyl acetate (2×100 mL). The organic extracts were combined, washed with brine (2×50 mL), dried over magnesium sulfate, filtered and the solvent removed in vacuo to give 666 mg (92% yield) of the title compound as a sticky gum which was used in the next step without further purification. ¹H NMR (500 MHz, CDCl₃) δ ppm 9.33 (br. s., 1H) 7.27 (d, J=7.93 Hz, 1H) 7.24 (d, J=7.78 Hz, 1H) 7.09 (t, J=7.93 Hz, 1H) 6.90 (d, J=8.39 Hz, 1H) 5.81 (m, J=17.01, 10.34, 6.54, 6.54 Hz, 1H) 5.07 (dd, J=17.09, 1.53 Hz, 1H) 5.02 (dd, J=10.15, 1.14 Hz, 1H) 4.88 (dd, J=8.39, 4.12 Hz, 1H) 4.69-4.80 (m, 1H) 2.63-2.76 (m, 2H) 2.39 (q, J=7.17 Hz, 2H) 2.30-2.36 (m, 1H) 1.54 (d, J=7.02 Hz, 6H) 1.07 (d, J=6.87 Hz, 3H) 0.88 (d, J=6.87 Hz, 3H). LC-MS: purity 89% (UV), t_{R} 2.07 min m/z [M+H]⁺ 358.05 (MET/CR/1278).

### Stage 2d:

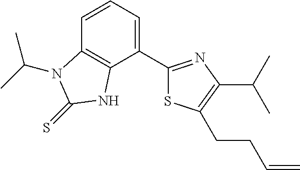

Stage 1d intermediate (592 mg, 1.66 mmol, 1.0 eq.), Lawesson reagent (803 mg, 1.99 mmol, 1.2 eq.) and dioxane (6 mL) were charged into a microwave tube. The reaction mixture was irridiated inside a Focus microwave (100 W, 180° C.) for 30 minutes. The solvent was removed in vacuo and the residue purified by flash column chromatography using a ethyl acetate/heptanes gradient to give 350 mg (57% yield) of the title compound as a brown solid. ¹H NMR (500 MHz, CDCl₃) δ ppm 11.21 (br. s., 1H) 7.46 (d, J=7.77 Hz, 1H) 7.38 (d, J=8.22 Hz, 1H) 7.18 (m, J=7.92, 7.92 Hz, 1H) 5.86 (m, J=17.00, 10.26, 6.62, 6.62 Hz, 1H) 5.65 (dt, J=14.12, 7.02 Hz, 1H) 5.00-5.16 (m, 2H) 3.12 (quin, J=6.81 Hz, 1 H) 2.92 (t, J=7.54 Hz, 2H) 2.32-2.53 (m, 2H) 1.61 (d, J=7.01 Hz, 6H) 1.37 (d, J=6.70 Hz, 6H). LC-MS: purity 91% (UV), t_{R} 2.51 min m/z [M+H]⁺ 372.45 (MET/CR/1278).

### Stage 3d:

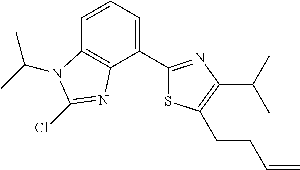

Stage 2d intermediate (350 mg, 0.941 mmol, 1.0 eq.) and phosphorous oxychloride (5 mL) were charged into a 10 mL round bottom flask and the reaction mixture heated at 111° C. for 15 hours. The aqueous phase was neutralised with saturated aqueous sodium hydrogencarbonate solution and extracted with ethyl acetate (100 mL). The organic extract was washed with saturated aqueous sodium hydrogencarbonate solution (50 mL), water (50 mL) and brine (50 mL), dried over magnesium sulfate, filtered and the solvent removed in vacuo. The residue was purified by flash column chromatography using a ethyl acetate/heptanes gradient to give 165 mg (47% yield) of the desired compound as a pale brown solid. ¹H NMR (500 MHz, CDCl₃) δ ppm 8.22 (d, J=7.32 Hz, 1H) 7.48 (dd, J=8.16, 0.84 Hz, 1H) 7.33 (t, J=7.93 Hz, 1H) 5.89 (m, J=17.01, 10.30, 6.64, 6.64 Hz, 1H) 5.09 (dd, J=17.01, 4.65 Hz, 1H) 5.00-5.05 (m, 1H) 4.96 (s, 1H) 3.12 (spt, J=6.82 Hz, 1H) 2.90-2.96 (m, 2H) 2.45 (td, J=7.78, 6.56 Hz, 2H) 1.69 (d, J=7.02 Hz, 6H) 1.35 (s, 6H). LC-MS: purity 100% (UV), t_{R} 2.46 min m/z [M+H]⁺ 374.45 (MET/CR/1278).

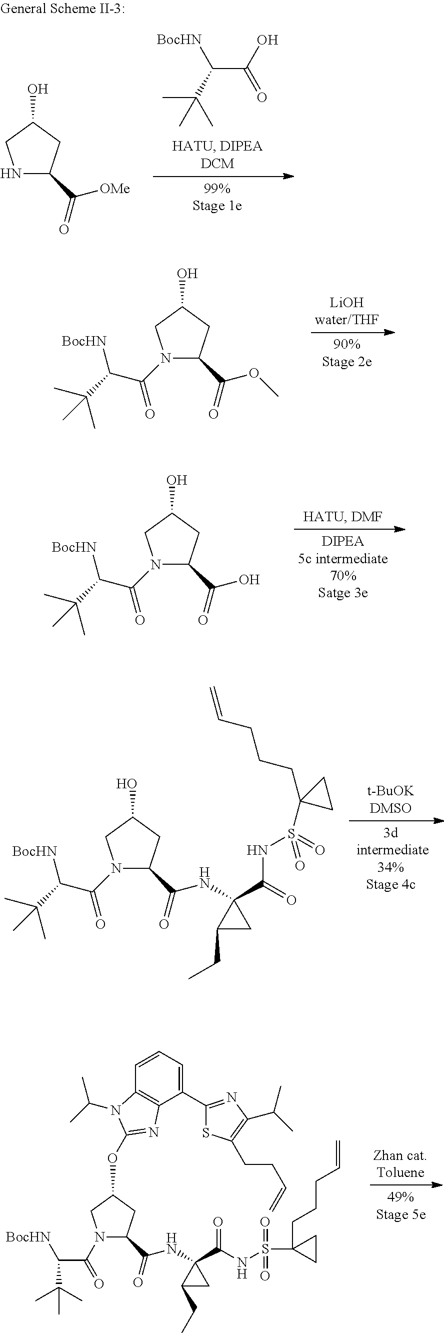

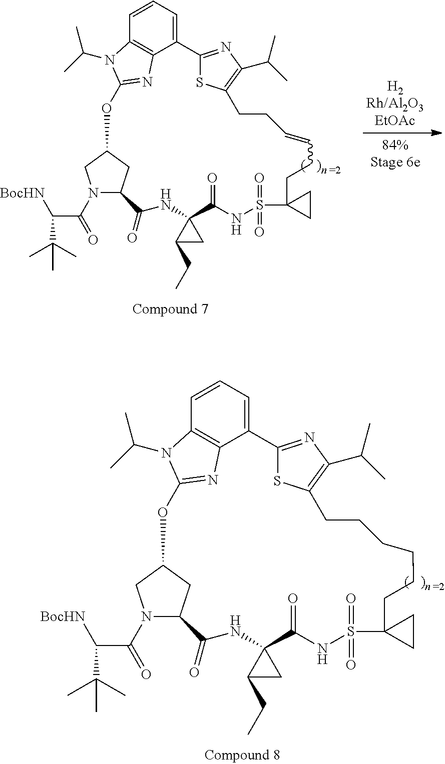

### Example II-3

### Stage 1e:

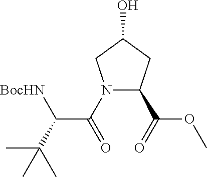

N-Boc-tert-leucine (5.00 g, 21.61 mmol, 1.0 eq.) and dichloromethane (70 mL) were charged into a 250 mL round bottom flask. HATU (12.32 g, 32.42 mmol, 1.5 eq.) was added as a single portion and the reaction mixture stirred at ambient temperature for 15 minutes then cooled to 0° C. Diisopropylethylamine (18.8 mL, 108.1 mmol, 5.0 eq.) was added as a single portion followed by 4-hydroxy-proline methyl ester (3.90 g, 21.61 mmol, 1.0 eq.). The reaction mixture was left to warm up to ambient temperature and stirring was continued for another 15 hours. The reaction mixture was washed with 5% aqueous citric acid solution (2×100 mL), 1M sodium hydroxide solution (2×100 mL) and brine (100 mL), dried over magnesium sulfate, filtered and the solvent removed in vacuo to give 7.8 g (99% yield) of the desired compound as a dark red oil which was used in the next step without further purification. ¹H NMR (500 MHz, CDCl₃) δ ppm 5.24 (d, J=9.31 Hz, 1H) 4.70 (t, J=8.62 Hz, 1H) 4.52 (br. s., 1H) 4.19 (d, J=9.46 Hz, 1H) 4.05 (d, J=10.99 Hz, 1H) 3.74 (s, 3H) 3.64-3.82 (m, 1H) 2.30-2.43 (m, 1H) 2.00 (ddd, J=13.31, 9.12, 4.27 Hz, 1H) 1.78 (br. s., 1H) 1.41 (s, 9H) 1.00-1.10 (m, 9H). LC-MS: purity 50% (UV), t_{R} 1.73 min m/z [M+Na]⁺ 381.45 (MET/CR/1278).

### Stage 2e:

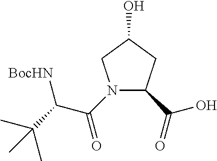

Stage 1e intermediate (7.8 g, 21.73 mmol, 1.0 eq.), water (100 mL), tetrahydrofuran (100 mL) and methanol (200 mL) were charged into a 1 L round bottom flask. Lithium hydroxide monohydrate (2.74 g, 65.19 mmol, 3.0 eq.) was added portion wise and the reaction mixture stirred at ambient temperature for 72 hours. The volatile solvents were removed in vacuo and 1 M sodium hydroxide solution (50 mL) was added to the residue (pH=14). The aqueous phase was washed with diethyl ether (2×100 mL) then acidified with 1 M hydrochloric acid until pH=4. The aqueous phase was extracted with ethyl acetate (3×150 mL). The organic extracts were combined, washed with water (100 mL) and brine (200 mL), dried over magnesium sulfate, filtered and the solvent removed in vacuo to give 6.7 g (90% yield) of the desired compound as a beige solid which was used in the next step without further purification. ¹H NMR (500 MHz, CDCl₃) δ ppm 5.34 (d, J=9.16 Hz, 1H) 4.76 (t, J=8.54 Hz, 1H) 4.52 (br. s., 1H) 4.27 (d, J=9.16 Hz, 1H) 4.17 (d, J=11.60 Hz, 1H) 3.63 (dd, J=11.60, 3.05 Hz, 1H) 2.81 (s, 1H) 2.27-2.43 (m, 2H) 1.42 (s, 9H) 1.03 (s, 9H). LC-MS: purity 68% (UV), t_{R} 1.60 min m/z [M+Na]⁺ 367.10 (MET/CR/1278).

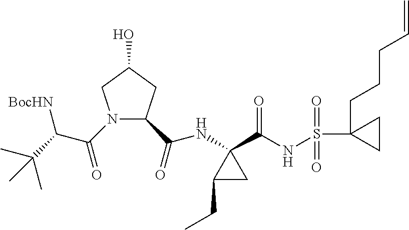

### Stage 3e:

Stage 2e intermediate (619 mg, 1.80 mmol, 1.0 eq.) and N,N-dimethylformamide (12 mL) were charged in a 25 mL round bottom flask. HATU (1030 mg, 2.70 mmol, 1.5 eq.) was added as a single portion and the reaction mixture stirred at ambient temperature for 15 minutes then cooled to 0° C. Diisopropylethylamine (1.6 mL, 9.0 mmol, 5.0 eq.) was added as a single portion followed by stage 5c intermediate (540 mg, 1.80 mmol, 1.0 eq.). The reaction mixture was left to warm up to ambient temperature and stirring was continued for another 15 hours. The reaction mixture was diluted with water (40 mL) and the pH adjusted to 3 with 1M aqueous hydrochloric acid. The aqueous phase was further extracted with ethyl acetate (2×30 mL). The organic extracts were combined, washed with water (5×75 mL), dried over magnesium sulfate, filtered and the solvent removed in vacuo. The residue was purified by flash column chromatography using a methanol/dichloromethane gradient to give 768 mg (70% yield) of the title compound as a beige solid. ¹H NMR (500 MHz, CDCl₃) δ ppm 9.78 (br. s., 1H) 7.47 (br. s., 1H) 5.70-5.84 (m, 1H) 5.21 (d, J=9.00 Hz, 1H) 5.01 (dd, J=17.17, 1.60 Hz, 1H) 4.97 (d, J=10.68 Hz, 1H) 4.59 (t, J=8.24 Hz, 1H) 4.53 (br. s., 1H) 4.19 (d, J=9.16 Hz, 1H) 4.13 (d, J=11.14 Hz, 1H) 3.55-3.66 (m, 1H) 2.40 (ddd, J=13.35, 8.93, 4.27 Hz, 1H) 2.16 (dd, J=13.66, 7.86 Hz, 1H) 2.06 (q, J=7.02 Hz, 2H) 1.89-1.99 (m, 1H) 1.78 (m, J=11.37, 5.26 Hz, 1H) 1.67-1.74 (m, 1H) 1.48-1.59 (m, 4 H) 1.44-1.47 (m, 1H) 1.42 (s, 9H) 1.27-1.37 (m, 1H) 1.07-1.14 (m, 1H) 1.04-1.07 (m, 2H) 1.02 (s, 8H) 0.98-1.01 (m, 3H) 0.87-0.97 (m, 3H). LC-MS: purity 100% (UV), t_{R} 2.29 min m/z [M+Na]⁺ 649.20 (MET/CR/1278).

### Stage 4e:

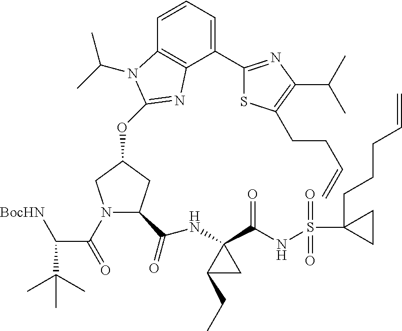

Stage 3e intermediate (277 mg, 0.441 mmol, 1.0 eq.) and dimethylsulfoxide (2 mL) were charged into a 12 mL vial. Potassium tert-Butoxide (198 mg, 1.76 mmol, 4.0 eq.) was added portionwise and the reaction mixture stirred at ambient temperature for 5 minutes. Stage 3d intermediate (165 mg, 0.441 mmol, 1.0 eq.) was dissolved in dimethylsulfoxide (1 mL) and the resulting solution added dropwise to the reaction mixture. Stirring was continued for another 4 hours, then the reaction mixture was diluted with water (2 mL) and the pH adjusted to 1 with 1M aqueous hydrochloric acid. The aqueous phase was extracted with ethyl acetate (3×25 mL). The organic extracts were combined, washed with water (3×25 mL), dried over magnesium sulfate, filtered and the solvent removed in vacuo. The residue was purified by flash column chromatography using a ethyl acetate/heptanes gradient to give 145 mg (34% yield) of the desired compound as a pale yellow solid. ¹H NMR (500 MHz, CDCl₃) δ ppm 9.78 (br. s., 1H) 8.13 (dd, J=7.63, 1.07 Hz, 1H) 7.23-7.26 (m, 1H) 7.20 (t, J=7.63 Hz, 1H) 7.15 (br. s., 1H) 5.85-5.95 (m, 2 H) 5.72-5.81 (m, 1H) 5.18-5.24 (m, 1H) 5.11 (dd, J=17.17, 1.45 Hz, 1H) 5.05 (br. s., 1 H) 5.00-5.04 (m, 1H) 4.93-5.00 (m, 1H) 4.55-4.63 (m, 2H) 4.32-4.38 (m, 1H) 4.29 (d, J=9.46 Hz, 1H) 4.12-4.19 (m, 1H) 3.12 (quin, J=6.75 Hz, 1H) 2.89-2.97 (m, 2H) 2.70-2.80 (m, 1H) 2.44 (q, J=7.38 Hz, 2H) 2.02-2.11 (m, 2H) 1.93-2.01 (m, 1H) 1.76-1.82 (m, 1H) 1.70-1.76 (m, 1H) 1.60-1.66 (m, 2H) 1.53 (d, J=6.87 Hz, 6H) 1.47-1.53 (m, 3 H) 1.40 (s, 9H) 1.36 (dd, J=6.87, 2.44 Hz, 6H) 1.24-1.32 (m, 2H) 1.12-1.19 (m, 1H) 1.04 (s, 9H) 1.01 (t, J=7.32 Hz, 3H) 0.87-0.94 (m, 3H). LC-MS: purity 100% (UV), t_{R} 5.46 min m/z [M+H]⁺ 964.35 (MET/CR/1426). HRMS: Found: 964.5035, calculated for C₅₀H₇₃N₇O₈S₂ (M+H)⁺: 964.5040.

### Stage 5e:

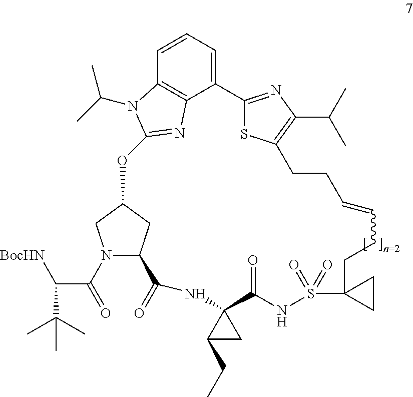

Stage 4e intermediate (140 mg, 0.142 mmol, 1.0 eq.) and toluene (50 mL, previously degased with nitrogen gas) were charged into a 100 mL round bottom flask and the reaction mixture heated to 65° C. Zhan catalyst (1.9 mg, 0.003 mmol, 2 mol %.) was added as a single portion and the reaction mixture stirred at 65° C. for 30 minutes. The reaction mixture was left to cool down to ambient temperature and the solvent removed in vacuo. The residue was purified by preparative HPLC to afford 65 mg (49% yield) of compound 7 as a white solid. ¹H NMR (500 MHz, CDCl₃) δ ppm 10.10 (br. s., 1H) 8.06-8.22 (m, 1H) 7.23-7.26 (m, 1H) 7.16-7.23 (m, 1H) 6.92 (s, 1H) 5.98 (br. s., 1H) 5.29-5.56 (m, 2H) 5.20 (d, J=9.16 Hz, 1H) 4.46-4.69 (m, 2H) 4.32 (d, J=9.61 Hz, 2H) 4.05-4.23 (m, 1H) 3.11 (dq, J=14.17, 6.92 Hz, 1H) 2.91-3.06 (m, 1H) 2.73-2.90 (m, 2H) 2.39-2.54 (m, 1H) 2.26-2.39 (m, 1H) 1.91-2.20 (m, 2H) 1.67-1.82 (m, 3H) 1.61-1.68 (m, 1H) 1.51-1.57 (m, 6H) 1.48-1.53 (m, 4H) 1.46 (s, 9H) 1.40-1.45 (m, 2H) 1.35-1.39 (m, 1H) 1.35 (d, J=6.71 Hz, 6H) 1.05 (s, 9H) 1.00-1.15 (m, 4H) 0.81-0.93 (m, 2H). LC-MS: purity 100% (UV), t_{R} 5.38 min m/z [M+H]⁺ 936.35 (MET/CR/1426). HRMS: Found: 936.4747, calculated for C₄₈H₆₉N₇O₈S₂ (M+H)⁺: 936.4727.

### Stage 6e:

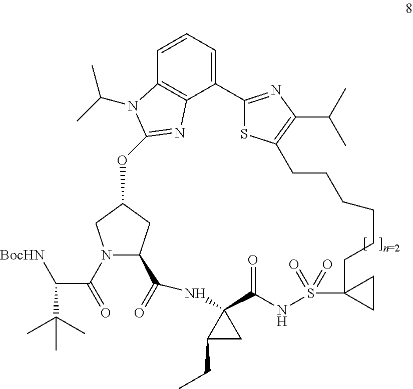

Compound 7 (58 mg, 0.062 mmol, 1.0 eq.), 5% rhodium on alumina (11.6 mg, 20 wt %) and ethyl acetate (5 mL) were charged into a 25 mL round bottom flask. The reaction mixture was placed under a hydrogen gas atmosphere and stirring was continued at ambient temperature for 15 hours. The catalyst was remove by filtration through glass fiber filter paper and the filtrate concentrated in vacuo to give 49.2 mg (84% yield) of compound 8 as an off-white solid. ¹H NMR (500 MHz, CDCl₃) δ ppm 9.79 (br. s., 1H) 8.15 (d, J=7.78 Hz, 1H) 7.23-7.29 (m, 1H) 7.18-7.23 (m, 0H) 6.97 (s, 1H) 5.93 (br. s., 1H) 5.21 (d, J=9.16 Hz, 1H) 4.55-4.67 (m, 2H) 4.27-4.39 (m, 2H) 4.07-4.19 (m, 2H) 3.11 (spt, J=6.87 Hz, 1H) 2.73-2.93 (m, 4H) 2.05 (s, 1H) 1.80-1.93 (m, 1H) 1.62-1.78 (m, 5H) 1.54-1.59 (m, 1H) 1.55 (d, J=6.87 Hz, 3H) 1.52 (d, J=6.87 Hz, 3H) 1.45 (s, 9H) 1.37-1.44 (m, 5H) 1.34 (d, J=6.87 Hz, 6H) 1.29-1.32 (m, 1H) 1.27 (t, J=7.17 Hz, 3H) 1.08-1.18 (m, 2H) 1.06 (s, 9H) 1.02 (t, J=7.10 Hz, 3H) 0.83-0.92 (m, 2H). LC-MS: purity 100% (UV), t_{R} 5.53 min m/z [M+H]⁺ 938.40 (MET/CR/1426). HRMS: Found: 938.4897, calculated for C₄₈H₇₁N₇O₈S₂ (M+H)⁺: 938.4884.

### Example II-4

### Preparation of Compound II-4a

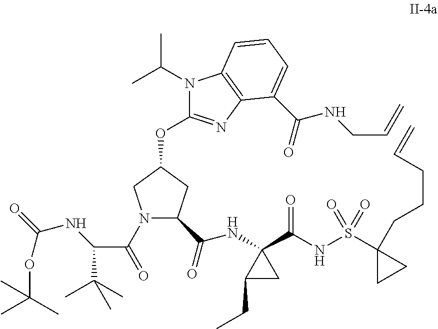

Diene II-4a was prepared following the method described for Stage 4e intermediate. 116 mg (19% yield) as a white solid after flash column chromatography. ¹H NMR (500 MHz, CDCl₃) δ ppm 9.85 (br. s., 1H) 9.49 (br. s., 1H) 8.03 (d, J=7.78 Hz, 1H) 7.56 (br. s., 1H) 7.36 (d, J=7.93 Hz, 1H) 7.21 (t, J=7.71 Hz, 1H) 5.99-6.13 (m, 1H) 5.69-5.83 (m, 2H) 5.38 (d, J=17.09 Hz, 1H) 5.25-5.33 (m, 1H) 5.20 (d, J=10.07 Hz, 1H) 5.01 (dd, J=17.17, 1.60 Hz, 1H) 4.96 (d, J=10.22 Hz, 1H) 4.60 (m, J=14.11, 7.40 Hz, 2H) 4.41 (d, J=11.90 Hz, 1H) 4.26 (d, J=9.46 Hz, 1H) 4.20 (br. s., 2H) 4.01 (dd, J=11.75, 3.51 Hz, 1H) 2.60-2.77 (m, 2H) 2.01-2.10 (m, 2H) 1.92-2.01 (m, 1H) 1.67-1.86 (m, 3H) 1.57-1.65 (m, 2H) 1.47-1.57 (m, 10H) 1.36-1.40 (m, 9H) 1.17 (dd, J=8.93, 5.57 Hz, 1H) 0.98-1.11 (m, 12H) 0.87-0.96 (m, 2H). LC-MS: purity 97% (UV), t_{R} 2.29 min m/z [M+H]⁺ 868.45 (MET/CR/1981).

### Preparation of Compound 9

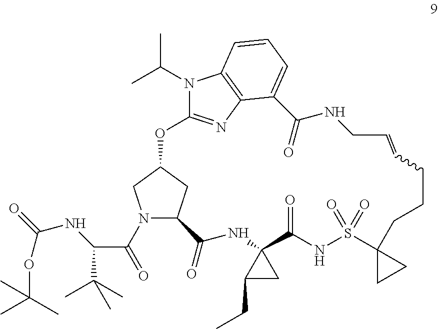

Compound 9 was prepared following the method described for compound 7 affording 13 mg (22% yield) as a white solid after preparative HPLC. ¹H NMR (500 MHz, CDCl₃) δ ppm 9.33-9.65 (m, 2H) 8.05 (d, J=7.48 Hz, 1H) 7.40 (d, J=8.09 Hz, 1H) 7.20-7.26 (m, 2H) 5.59-5.81 (m, 3H) 5.13-5.25 (m, 1H) 4.58-4.72 (m, 2H) 4.44-4.58 (m, 1 H) 4.31-4.39 (m, 1H) 4.23-4.30 (m, 1H) 4.00-4.17 (m, 1H) 3.57-3.80 (m, 1H) 2.75-2.85 (m, 2H) 2.06-2.22 (m, 1H) 1.94-2.06 (m, 1H) 1.76-1.91 (m, 1H) 1.58-1.71 (m, 2 H) 1.51-1.58 (m, 6H) 1.48-1.51 (m, 2H) 1.46 (s, 9H) 1.29-1.43 (m, 3H) 1.18-1.29 (m, 1H) 1.05-1.08 (m, 2H) 1.04 (s, 9H) 0.98-1.03 (m, 3H) 0.72-0.83 (m, 1H) 0.48-0.61 (m, 1H). LC-MS: purity 100% (UV), tR 4.45 min m/z [M+H]⁺ 840.55 (MET/CR/1426). HRMS: Found: 840.4337, calculated for C₄₂H₆₁N₇O₉S (M+H)⁺: 840.4330.

### Preparation of Compound II-4b

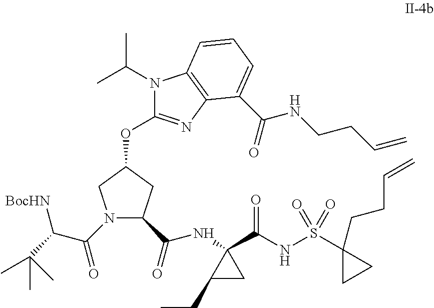

Diene II-4b was prepared following the method described for Stage 4e intermediate. 110 mg (32% yield) as a white solid after preparative HPLC. ¹H NMR (500 MHz, CDCl₃) δ ppm 9.76 (br. s., 1H) 9.29-9.42 (m, 1H) 8.06 (d, J=7.63 Hz, 1H) 7.37 (d, J=7.93 Hz, 1H) 7.23 (t, J=7.86 Hz, 1H) 5.87-6.03 (m, 1H) 5.72-5.85 (m, 2H) 5.15-5.25 (m, 2H) 5.10 (d, J=10.83 Hz, 1H) 5.05 (dd, J=17.17, 1.45 Hz, 1H) 4.97 (d, J=9.92 Hz, 1H) 4.53-4.65 (m, 2H) 4.39 (d, J=11.75 Hz, 1H) 4.25 (d, J=9.46 Hz, 1H) 4.00 (dd, J=11.98, 4.04 Hz, 1H) 3.60-3.75 (m, 2H) 2.59-2.77 (m, 2H) 2.45 (q, J=6.56 Hz, 2H) 2.12-2.30 (m, 2H) 2.00-2.11 (m, 1H) 1.80-1.93 (m, 1H) 1.69-1.78 (m, 1H) 1.61 (s, 5H) 1.52 (d, J=6.87 Hz, 6H) 1.38 (s, 9H) 1.16 (dd, J=9.31, 5.80 Hz, 1H) 1.04 (s, 9H) 0.90-1.02 (m, 6 H). LC-MS: purity 100% (UV), tR 5.41 min m/z [M+H]⁺ 868.25 (MET/CR/1416). HRMS: Found: 868.4637, calculated for C₄₄H₆₅N₇O₉S (M+H)⁺: 868.4643.

### Preparation of Compound 10

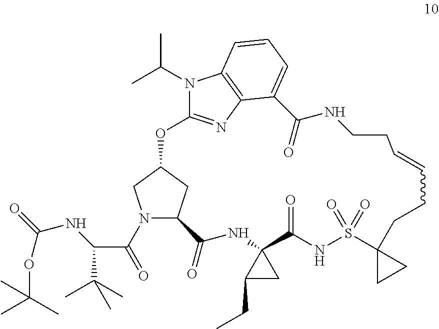

Compound 10 was prepared using Diene II-4b following the method described for compound 7 affording 27 mg (31% yield) as a white solid after preparative HPLC. ¹H NMR (500 MHz, CDCl₃) δ ppm 10.13 (br. s., 1H) 9.30-9.53 (m, 1H) 8.00 (d, J=7.78 Hz, 1H) 7.36 (d, J=7.93 Hz, 1H) 7.20 (t, J=7.93 Hz, 1H) 7.09 (s, 1H) 5.62-5.66 (m, 1H) 5.54-5.62 (m, 1H) 5.38-5.49 (m, 1H) 5.21 (d, J=9.46 Hz, 1H) 4.58 (spt, J=6.87 Hz, 1H) 4.44 (d, J=11.75 Hz, 1H) 4.39 (t, J=8.16 Hz, 1H) 4.26 (d, J=9.46 Hz, 1H) 4.05 (dd, J=11.67, 3.89 Hz, 1H) 3.71-3.82 (m, 1H) 3.34 (ddd, J=9.99, 6.71, 3.13 Hz, 1H) 2.93 (dd, J=14.50, 7.02 Hz, 1H) 2.57 (ddd, J=14.08, 9.65, 4.20 Hz, 1H) 2.41-2.52 (m, 1H) 2.27-2.38 (m, 1H) 2.15-2.27 (m, 1H) 1.96-2.08 (m, 1H) 1.80-1.92 (m, 1H) 1.55-1.63 (m, 2H) 1.53 (d, J=6.87 Hz, 3H) 1.50 (d, J=7.02 Hz, 3H) 1.43-1.45 (m, 2H) 1.47 (br. s., 9H) 1.36-1.43 (m, 3H) 1.21 (dd, J=8.39, 5.04 Hz, 1H) 1.04 (s, 9H) 1.00 (t, J=7.17 Hz, 3H) 0.66-0.76 (m, 1H) 0.27-0.39 (m, 1H). LC-MS: purity 100% (UV), t_{R} 4.44 min m/z [M+H]⁺ 840.35 (MET/CR/1426). HRMS: Found: 840.4333, calculated for C₄₂H₆₁N₇O₉S (M+H)⁺: 840.4330.

### Preparation of Compound 11

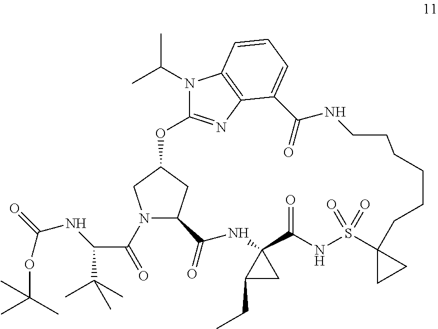

Compound 11 was prepared by hydrogenating compound 10 following the method described for compound 8 affording 19 mg (95% yield) as a white solid after preparative HPLC. ¹H NMR (500 MHz, CDCl₃) δ ppm 9.71 (br. s., 1H) 9.40-9.51 (m, 1H) 8.02 (d, J=7.78 Hz, 1H) 7.34-7.41 (m, 2H) 7.22 (t, J=7.93 Hz, 1H) 5.70-5.79 (m, 1H) 5.21 (d, J=9.46 Hz, 1H) 4.56-4.66 (m, 2H) 4.33 (d, J=11.14 Hz, 1H) 4.27 (d, J=9.61 Hz, 1 H) 4.08 (dd, J=11.52, 4.96 Hz, 1H) 3.75-3.86 (m, 1H) 3.16-3.27 (m, 1H) 2.70-2.86 (m, 2H) 1.76-1.83 (m, 1H) 1.57-1.67 (m, 5H) 1.54 (d, J=6.87 Hz, 3H) 1.52 (d, J=7.02 Hz, 3H) 1.47-1.50 (m, 2H) 1.46 (s, 9H) 1.35-1.44 (m, 4H) 1.28-1.34 (m, 4H) 1.17-1.23 (m, 1H) 1.03 (s, 9H) 1.00 (t, J=7.32 Hz, 3H) 0.69-0.78 (m, 1H) 0.43-0.53 (m, 1H). LC-MS: purity 100% (UV), t_{R} 5.33 min m/z [M+H]⁺ 842.40 (MET/CR/1416). HRMS: Found: 842.4497, calculated for C₄₂H₆₃N₇O₉S (M+H)⁺: 842.4486.

### Preparation of Compound II-4c

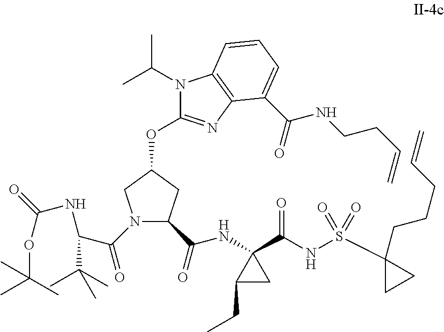

Diene II-4c was prepared following the method described for Stage 4e intermediate. 500 mg (27% yield) as an off-white solid after flash column chromatography. ¹H NMR (500 MHz, CDCl₃) δ ppm 9.80 (s, 1H) 9.33-9.42 (m, 1H) 8.04 (d, J=7.63 Hz, 1 H) 7.35 (d, J=7.93 Hz, 1H) 7.18-7.25 (m, 2H) 5.88-5.99 (m, 1H) 5.68-5.85 (m, 2H) 5.18-5.23 (m, 1H) 5.08-5.15 (m, 1H) 5.00 (dd, J=17.09, 1.68 Hz, 1H) 4.95 (d, J=10.07 Hz, 1H) 4.52-4.67 (m, 2H) 4.38 (d, J=12.05 Hz, 1H) 4.27 (d, 1H) 4.01 (dd, J=11.83, 3.89 Hz, 1H) 3.61-3.73 (m, 1H) 3.49-3.60 (m, 1H) 2.61-2.77 (m, 2H) 2.35-2.49 (m, 3H) 2.01-2.10 (m, 2H) 1.91-2.01 (m, 1H) 1.71-1.81 (m, 1H) 1.56-1.65 (m, 2H) 1.52 (dd, J=17.85, 7.02 Hz, 12H) 1.37 (s, 9H) 1.12-1.19 (m, 1H) 1.04 (s, 9H) 0.97-1.02 (m, 3H) 0.91-0.96 (m, 2H). LC-MS: purity 71% (UV), t_{R} 2.34 min m/z [M+H]⁺ 882.20 (MET/CR/1981).

### Preparation of Compound 12

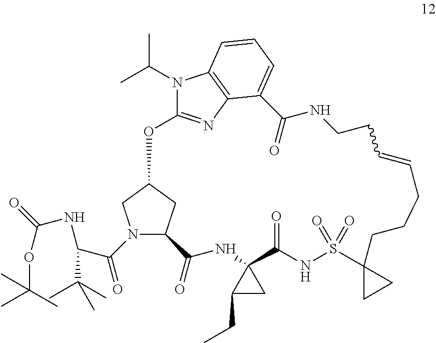

Compound 12 was prepared using Diene II-4c following the method described for compound 7 affording 133 mg (39% yield) as an off-white solid after preparative HPLC. ¹H NMR (500 MHz, CDCl₃) δ ppm 9.99 (br. s., 1H) 9.30-9.52 (m, 1H) 8.05 (d, J=7.78 Hz, 1H) 7.39 (d, J=7.93 Hz, 1H) 7.18-7.27 (m, 2H) 5.83 (br. s., 1H) 5.45-5.64 (m, 2H) 5.20 (d, J=9.31 Hz, 1H) 4.57-4.71 (m, 2H) 4.34 (d, J=11.29 Hz, 1H) 4.29 (d, J=9.46 Hz, 1H) 4.02-4.10 (m, 1H) 3.77-3.90 (m, 1H) 3.50 (d, J=5.34 Hz, 1H) 3.32-3.43 (m, 1H) 2.84-2.97 (m, 1H) 2.54-2.64 (m, 1H) 2.45-2.54 (m, 1H) 2.29-2.40 (m, 1 H) 1.88-2.01 (m, 2H) 1.67-1.79 (m, 2H) 1.63-1.66 (m, 1H) 1.48-1.59 (m, 10H) 1.46 (s, 9H) 1.34-1.44 (m, 2H) 1.08-1.13 (m, 1H) 1.05 (s, 9H) 1.00-1.04 (m, 3H) 0.82-0.89 (m, 2H). LC-MS: purity 100% (UV), t_{R} 4.47 min m/z [M+H]⁺ 854.25 (MET/CR/1416). HRMS: Found: 854.4488, calculated for C₄₃H₆₃N₇O₉S (M+H)⁺: 854.4486.

### Preparation of Compound 13

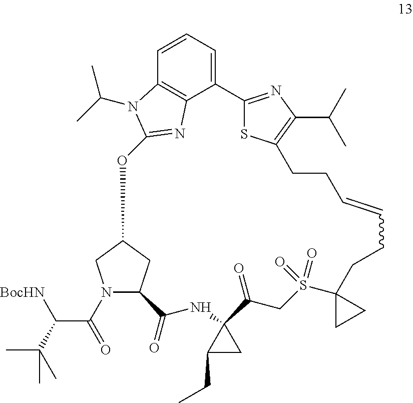

Compound 13 was prepared following the method described for compound 7 affording 41 mg (37% yield) as an off-white solid after preparative HPLC. ¹H NMR (500 MHz, CDCl₃) δ ppm 9.02-10.08 (m, 1H) 8.14 (d, J=7.48 Hz, 1H) 7.17-7.27 (m, 2H) 6.84 (s, 1H) 5.82-5.95 (m, 1H) 5.29-5.64 (m, 2H) 5.14-5.26 (m, 1H) 4.56-4.70 (m, 1H) 4.23-4.48 (m, 2H) 4.08-4.23 (m, 1H) 3.08-3.18 (m, 1H) 2.99-3.08 (m, 1 H) 2.84-2.99 (m, 1H) 2.71-2.84 (m, 1H) 2.40-2.61 (m, 2H) 2.18-2.38 (m, 2H) 1.75-1.87 (m, 1H) 1.65-1.75 (m, 2H) 1.51-1.58 (m, 6H) 1.49-1.54 (m, 4H) 1.42-1.49 (m, 11H) 1.29-1.40 (m, 7H) 0.97-1.13 (m, 13H) 0.81-0.96 (m, 2H). LC-MS: purity 100% (UV), t_{R} 5.26 min m/z [M+H]⁺ 922.20 (MET/CR/1426). HRMS: Found: 922.4579, calculated for C₄₇H₆₇N₇O₈S₂ (M+H)⁺: 922.4571.

### Preparation of Compound 14

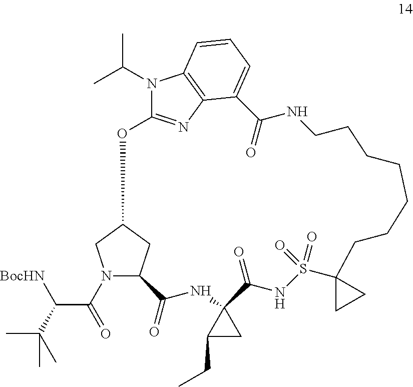

Compound 14 was prepared by hydrogenating compound 12 following the method described for compound 8 affording 98 mg (89% yield) as an off-white solid. ¹H NMR (500 MHz, CDCl₃) δ ppm 9.93 (br. s., 1H) 9.33-9.43 (m, 1H) 8.05 (d, J=7.72 Hz, 1 H) 7.40 (d, J=7.88 Hz, 1H) 7.24 (t, J=7.88 Hz, 1H) 7.07 (s, 1H) 5.81 (br. s., 1H) 5.20 (d, J=9.30 Hz, 1H) 4.59-4.69 (m, 2H) 4.41 (d, J=11.82 Hz, 1H) 4.30 (d, J=9.30 Hz, 1H) 4.01 (dd, J=11.66, 4.10 Hz, 1H) 3.81-3.91 (m, 1H) 3.24-3.32 (m, 1H) 2.76-2.84 (m, 1H) 2.67-2.74 (m, 1H) 1.63-1.84 (m, 5H) 1.52-1.56 (m, 6H) 1.49-1.54 (m, 4H) 1.45 (s, 9 H) 1.39-1.45 (m, 4H) 1.21-1.38 (m, 4H) 1.12-1.18 (m, 1H) 1.06 (s, 9H) 0.99-1.04 (m, 4H) 0.82-0.91 (m, 2H). LC-MS: purity 94% (UV), t_{R} 4.57 min m/z [M+H]⁺ 856.30 (MET/CR/1426). HRMS: Found: 856.4630, calculated for C₄₃H₆₅N₇O₉S (M+H)⁺: 856.4643.

### Preparation of Compound 15

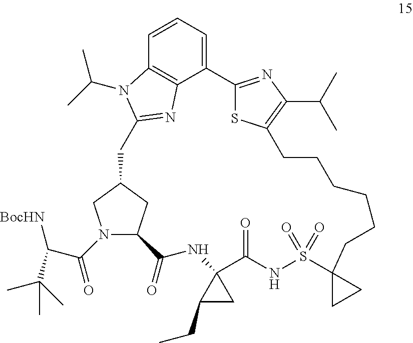

Compound 15 was prepared by hydrogenating compound 13 following the method described for compound 8 affording 12.5 mg (35% yield) as a yellow solid after preparative HPLC. ¹H NMR (500 MHz, CDCl₃) δ ppm 9.70 (br. s., 1H) 8.14 (d, J=7.72 Hz, 1H) 7.23-7.27 (m, 2H) 7.20 (t, J=7.72 Hz, 1H) 7.10 (s, 1H) 5.89 (br. s., 1H) 5.20 (d, J=9.46 Hz, 1H) 4.57-4.67 (m, 2H) 4.33 (d, J=9.77 Hz, 2H) 4.08-4.18 (m, 1H) 3.11 (dt, J=13.67, 6.80 Hz, 1H) 2.93-2.99 (m, 2H) 2.85-2.93 (m, 1H) 2.73-2.83 (m, 1H) 1.65-1.83 (m, 6H) 1.54 (dd, J=14.03, 6.94 Hz, 6H) 1.51-1.53 (m, 2H) 1.46 (s, 9H) 1.39-1.44 (m, 6H) 1.34 (d, J=6.78 Hz, 6H) 1.24-1.29 (m, 1H) 1.10-1.17 (m, 1H) 1.05 (s, 9H) 1.01-1.04 (m, 3H) 0.78-0.89 (m, 2H). LC-MS: purity 100% (UV), t_{R} 5.41 min m/z [M+H]⁺924.30 (MET/CR/1426). HRMS: Found: 924.4737, calculated for C₄₇H₆₉N₇O₈S₂ (M+H)⁺: 924.4727.

### Preparation of Compounds 101-104

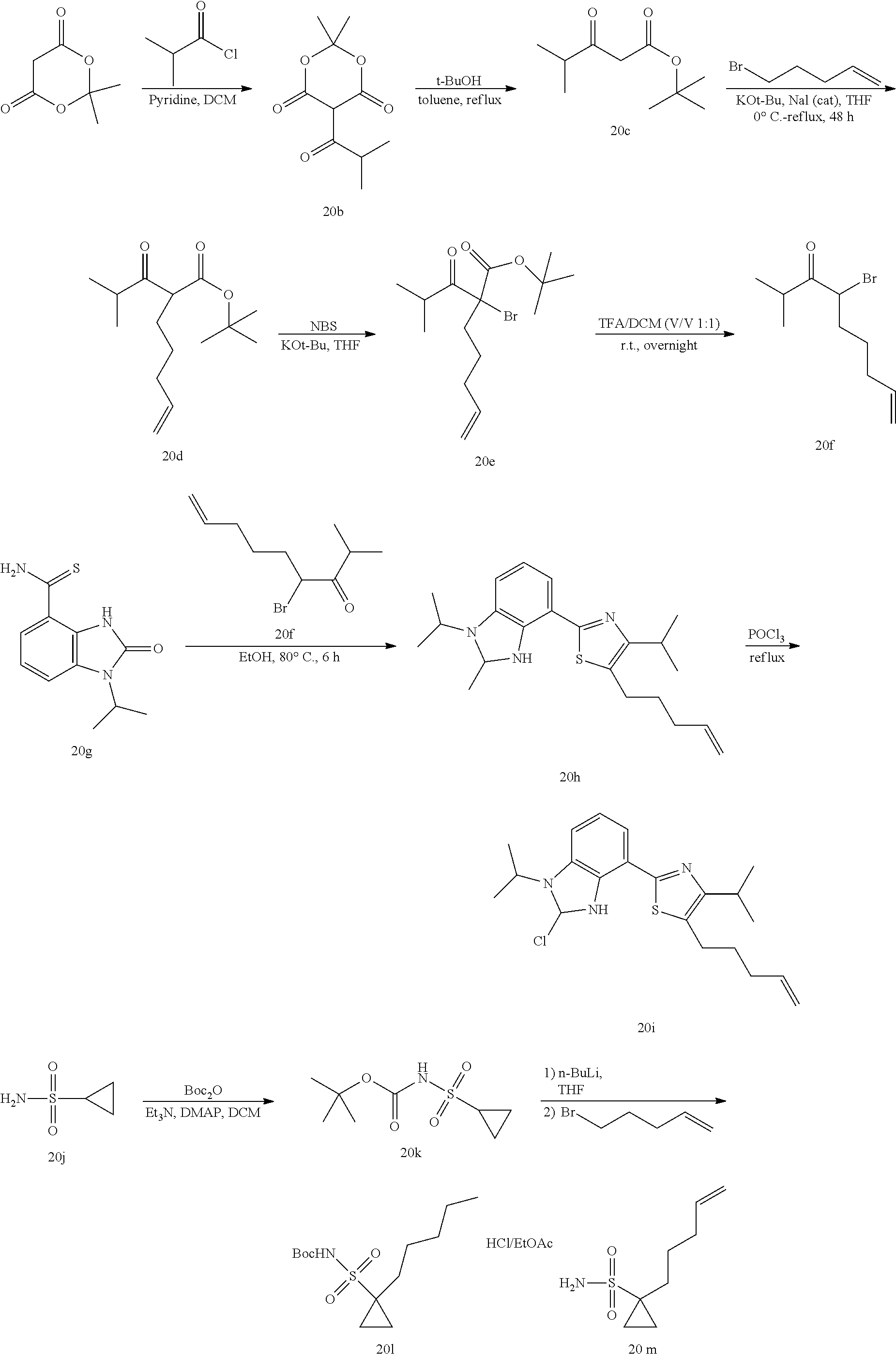

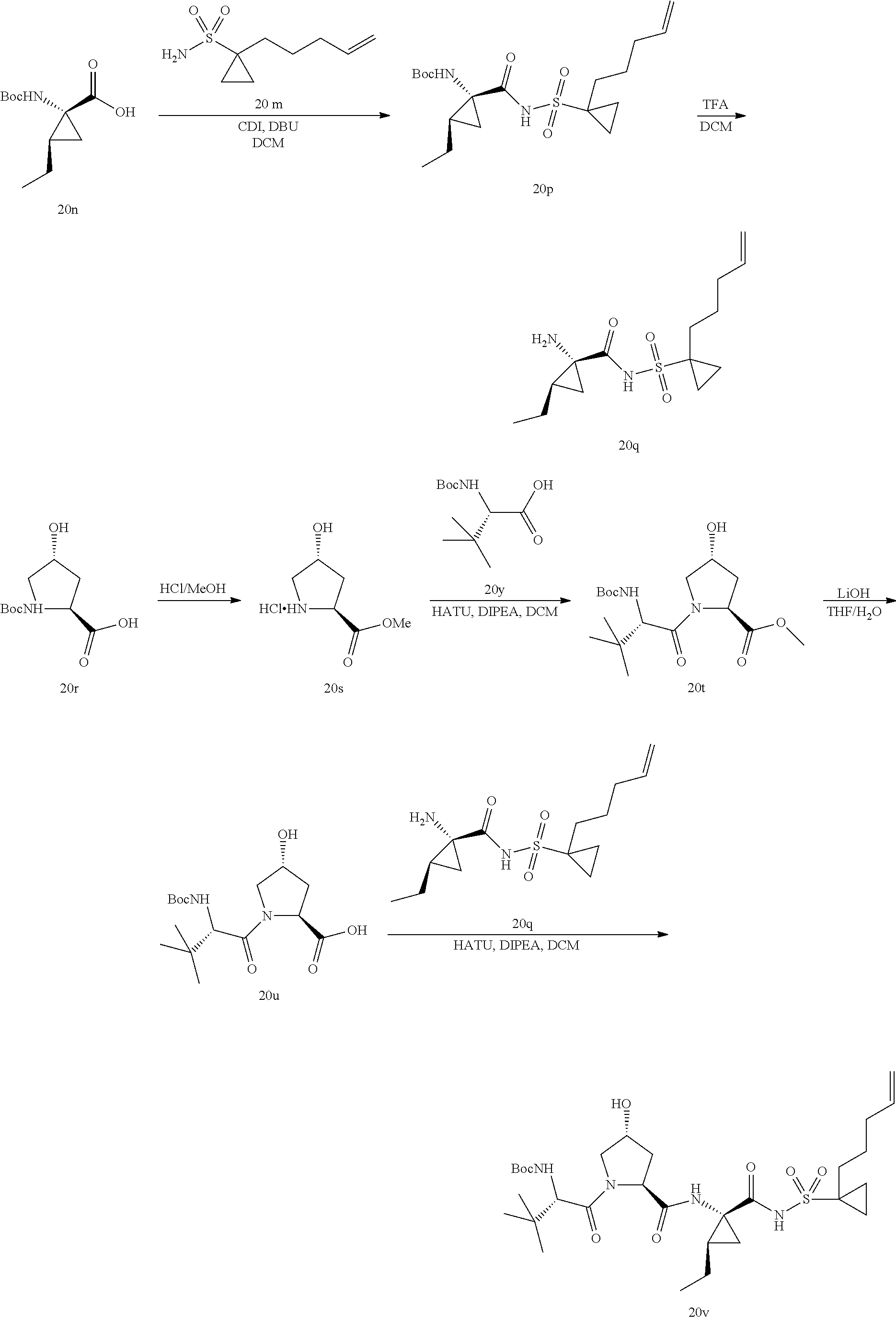

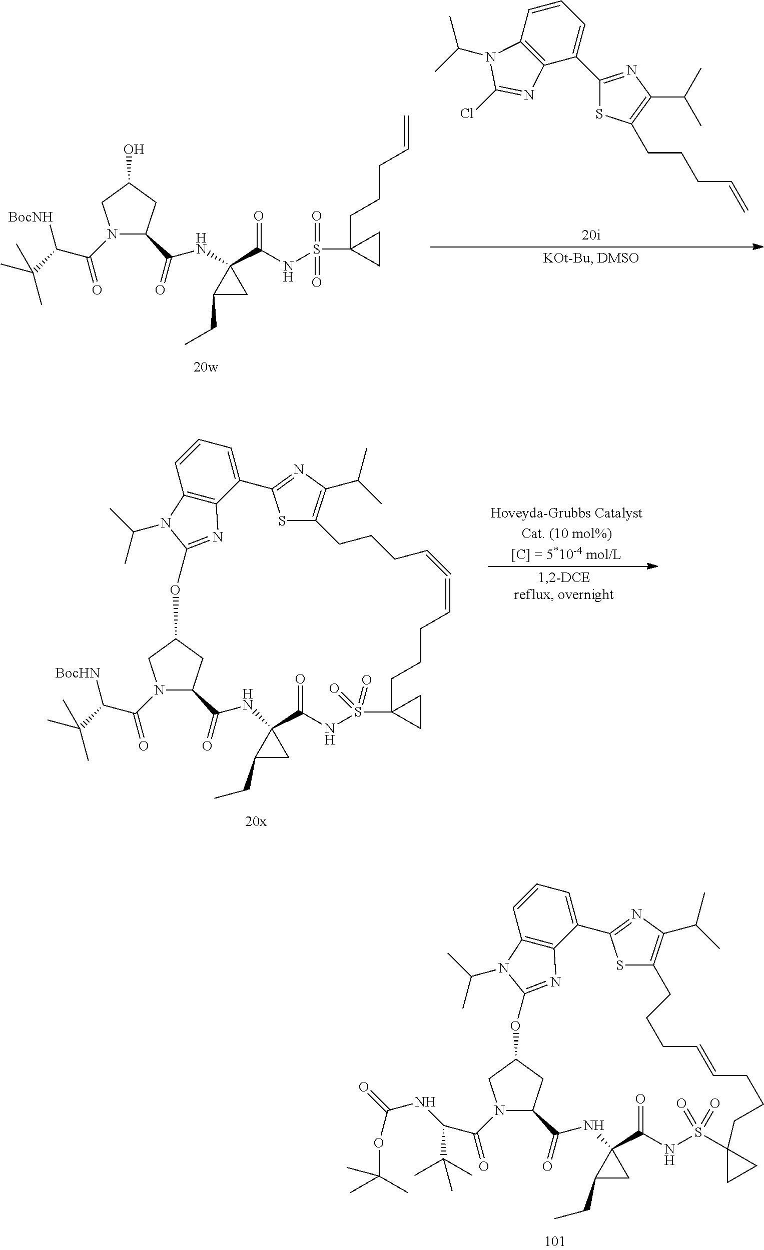

To a solution of compound 20a (93.2 g, 0.64 mol) in DCM (500 mL) was added pyridine (125 mL, 1.53 mol) at 0° C. And then a solution of isobutyryl chloride (25 g, 0.235 mol) in DCM (100 mL) was added. The mixture was stirred for 1 hour at 0° C., then warmed to room temperature and stirred for additional 1 hour. The reaction mixture was diluted with DCM (300 mL), washed with aq.HCl (2 M) and brine, dried over Na₂SO₄ and concentrated in vacuo to provide compound 20b (75.6 g, crude yield 148%) as a brawn oil, which was used directly in next step.

To a solution of compound 20b (75.6 g, 0.35 mol) in toluene (200 mL) was added t-BuOH (60 g, 0.81 mol) portion-wise. The mixture was heated to reflux for 30 hrs. Then the reaction mixture was concentrated in vacuo. The resulting residue was purified by column chromatography on silica gel (eluted with petroleum ether) to give compound 20c (39 g, yield 60%) as a light yellow oil.

To a solution of compound 20c (36 g, 0.2 mol) in THF (450 mL) was added KOt-Bu (25 g, 0.22 mol) portion-wise at 0° C. After stirring for 1 hour, NaI (15 g, 0.1 mol) and 5-Bromo-pent-1-ene (34 g, 0.22 mol) was added in one portion. And then the mixture was heated to reflux for 48 hrs. The reaction mixture was diluted with EtOAc (300 mL), the organic layer was washed with water and brine, and then dried over Na₂SO₄, concentrated in vacuo. The residue was purified by column chromatography on silica gel (petroleum ether/EtOAc=100/1) to afford compound 20d (24 g, yield 49%) as a light yellow oil.

¹H NMR (300 MHz, CDCl₃): δ 5.80-5.64 (m, 1H), 4.94 (d, J=18 Hz, 1H), 4.90 (d, J=10.8 Hz, 1H), 3.46 (t, J=6.9 Hz, 1H), 2.78-2.69 (m, 1H), 2.06-1.97 (m, 2H), 1.78-1.70 (m, 2H), 1.39 (s, 9H), 1.35-1.28 (m, 2H), 1.05 (t, J=6.0 Hz, 6H). MS (ESI) m/z (M+Na)⁺ 277.1.

To a solution of compound 20d (15 g, 29.5 mmol) in THF (200 mL) was added KOt-Bu (7.94 g, 35.4 mmol) portion-wise at 0° C. After stirring for 30 min, NBS (11.5 g, 32.4 mol) was added in one portion. The mixture was allowed to warm to ambient temperature and stirred for 12 hrs. The reaction mixture was diluted with EtOAc (200 mL), the organic layer was washed with water and brine, dried over Na₂SO₄, concentrated in vacuo. The residue was distilled to give compound 20e (5.6 g, yield 57%).

¹H NMR (300 MHz, CDCl₃): δ 5.82-5.69 (m, 1H), 5.00 (d, J=17.7 Hz, 1H), 4.96 (d, J=9.9 Hz, 1H), 3.10-3.01 (m, 1H), 2.18-2.05 (m, 4H), 1.56-1.35 (m, 2H), 1.46 (s, 9H), 1.22 (d, J=6.6 Hz, 3H), 1.13 (d, J=6.6 Hz, 3H).

Compound 20e (10 g, 30 mmol) was dissolved in DCM (50 mL), the resulting solution was cooled to 0° C. with an ice bath, and TFA (50 mL) was added dropwise thereto. The mixture was warmed to 40° C. and stirred for 24 hrs. The solution was concentrated in vacuo, then the residue was diluted with EtOAc (100 mL), the organic layer was washed with saturated aq.NaHCO₃ and brine, and then dried over Na₂SO₄. The solution was concentrated in vacuo and the residue was distilled to give compound 20f (3 g, yield 42%).

¹H NMR (400 MHz, CDCl₃): δ 5.81-5.70 (m, 1H), 5.00 (d, J=20 Hz, 1H), 4.97 (d, J=10.8 Hz, 1H), 4.38 (t, J=7.2 Hz, 1H), 3.06-2.98 (m, 1H), 2.11-2.02 (m, 2H), 2.00-1.87 (m, 2H), 1.57-1.50 (m, 1H), 1.46-1.38 (m, 1H), 1.16 (d, J=6.8 Hz, 3H), 1.12 (d, J=6.8 Hz, 3H).

A mixture of compound 20 g (1 g, 4.3 mmol) and compound 20f (1 g, 4.3 mmol) in EtOH (10 mL) was refluxed for 6 hrs. After the material was consumed, the reaction mixture was concentrated under reduced pressure. The residue was diluted with EtOAc (100 mL) and washed with brine. The organic layer was dried over Na₂SO₄, concentrated in vacuo. The resulting residue was purified by prep-TLC (PE/EtOAc=3/1) to afford compound 20 h as a light yellow solid (200 mg, yield 13%).

¹H NMR (400 MHz, CDCl₃): δ 9.73 (s, 1H), 7.32 (d, J=8.0 Hz, 1H), 7.11 (d, J=8.0 Hz, 1H), 7.05 (t, J=8.0 Hz, 1H), 5.88-5.78 (m, 1H), 5.06 (d, J=17.2 Hz, 1H), 5.02 (d, J=12 Hz, 1H), 4.79-4.74 (m, 1H), 3.12-3.05 (m, 1H), 2.80 (t, J=8.0 Hz, 2H), 2.18-2.11 (m, 2H), 1.79-1.70 (m, 2H), 1.55 (d, J=8.0 Hz, 6H), 1.33 (d, J=8.0 Hz, 6H). MS (ESI) m/z (M+H)⁺ 369.9.

Compound 20 h (150 mg, 0.41 mmol) was dissolved in POCl₃ (2 mL), the solution was heated to reflux for 12 hrs. After concentration, the residue was diluted with EtOAc (50 mL) and neutralized with saturated aq. NaHCO₃; the organic phase was separated, dried with Na₂SO₄, the solvent removed in vacuo to give a brown residue. The residue was purified by prep-TLC (PE/EtOAc=4/1) to give compound 20i as a light yellow solid (120 mg, yield 73%). MS (ESI) m/z (M+H)⁺ 388.1.

A round bottom flask (250 mL) was charged with cyclopropanesulfonamide 20j (6 g, 0.05 mol, 1.0 eq.), triethylamine (10 g, 0.08 mol, 1.6 eq.) and dichloromethane (100 mL). To the resulting mixture was added Di-tert-butyldicarbonate (13 g, 0.06 mol, 1.2 eq.) and DMAP (300 mg, 2.5 mmol, 0.05 eq.) in portions. The reaction mixture was stirred at ambient temperature for 12 hours. The solvent was removed in vacuo and the residue was diluted with water (50 mL). The aqueous phase was acidified to pH=3 with citric acid and extracted with ethyl acetate (100 mL×3). The organic layers were combined, washed with water and brine, dried over sodium sulfate, filtered and the solvent was removed in vacuo to give compound 20k (11 g, yield 99%) as a white solid.

¹H NMR (300 MHz, CDCl₃): δ 2.88 (m, 1H), 1.52 (s, 9H), 1.35 (m, 2H), 1.11 (m, 2H).

Compound 20k (4.42 g, 20 mmol, 1.0 eq.) and anhydrous tetrahydrofuran (40 mL) were charged into a round bottom flask (100 mL). The solution was cooled down to −78° C. n-BuLi (2.5 M, 20 mL, 50 mmol, 2.5 eq.) was added drop wise and stirring continued for 1.5 hrs −78° C. And then 5-Bromo-pent-1-ene (3.6 g, 24 mmol, 1.2 eq.) was added drop wise. The reaction mixture was left to warm up to −15° C. and stirring was continued for 30 min. The reaction mixture was quenched with saturated aqueous ammonium chloride solution and extracted with ethyl acetate (80 mL×3). The combined organic layer was washed with brine, dried over sodium sulfate, filtered and concentrated in vacuo to afford compound 201 (5.4 g, yield 94%) as a white solid, which was pure enough for the next step.

¹H NMR (400 MHz, DMSO-d₆): δ 11.00 (s, 1H), 5.82-5.70 (m, 1H), 4.99 (d, J=17.2 Hz, 1H), 4.94 (d, J=11.2 Hz, 1H), 2.03-1.95 (m, 2H), 1.78-1.71 (m, 2H), 1.55-1.46 (m, 2H), 1.41 (s, 9H), 1.34-1.30 (m, 2H), 0.95-0.90 (m, 2H).

Compound 201 (3.2 g, 11 mmol) was dissolved in EtOAc (25 mL). A solution of hydrogen chloride in EtOAc (4M, 5 mL) was added thereto. The reaction mixture was stirred at ambient temperature for 12 hrs. The solvent was concentrated in vacuo, the residue was diluted with EtOAc (100 mL) and neutralized with saturated aq. NaHCO₃. The organic layer was separated and washed with brine, dried over sodium sulfate, filtered and the solvent removed in vacuo to provide compound 20m (2.1 g, yield 99%) as a brown solid.

¹H NMR (300 MHz, CDCl₃): δ 5.84-5.70 (m, 1H), 5.01 (d, J=17.1 Hz, 1H), 4.96 (d, J=9.9 Hz, 1H), 4.66 (brs, 2H), 2.10-2.02 (m, 2H), 1.92-1.85 (m, 2H), 1.62-1.52 (m, 2H), 1.38-1.32 (m, 2H), 0.89-0.82 (m, 2H).

Compound 20n (526 mg, 2.3 mmol, 1.0 eq.) and dichloromethane (5 mL) were charged into a round bottom flask (25 mL). 1,1′-Carbonyldiimidazole (484 mg, 3.1 mmol, 1.3 eq.) was added and the reaction mixture stirred at reflux for 2 hrs. The resulting mixture was cooled to r.t., and then compound 20m (1000 mg, 5.3 mmol, 2.3 eq.) and DBU (874 mg, 5.7 mmol, 2.5 eq.) were added thereto. After that, the reaction mixture was heated to reflux and stirring was continued for 8 hrs. The reaction mixture was concentrated, diluted with water (20 mL) and adjusted to pH=4-5 with citric acid (aq.). The aqueous phase was extracted with EtOAc (30 mL×3). The combined organic layer was washed with brine, dried over sodium sulfate, filtered and the solvent removed in vacuo. The residue was purified by prep-TLC (petroleum ether/EtOAc=3/1) to yield compound 20p (360 mg, yield 39%) as a white solid. MS (ESI) m/z (M+H)⁺ 401.1.

Compound 20p (650 mg, 1.6 mmol) was dissolved in dichloromethane (6 mL). To the solution was added dropwise TFA (6 mL). The reaction mixture stirred at ambient temperature for 3 hrs. The solvent was removed in vacuo, the residue was diluted with EtOAc (100 mL) and neutralized with saturated aq. NaHCO₃. The organic layer was separated and washed with brine, dried over sodium sulfate, filtered and concentrated in vacuo to provide compound 20q (487 mg, yield 99%) as a beige solid. MS (ESI) m/z (M+H)⁺ 301.1.

Compound 20r (2 g, 8.6 mmol) was taken up with a solution of HCl (g) in methanol (4 M, 40 mL). The mixture was stirred at ambient temperature for 12 hrs. After that, the reaction mixture was concentrated under reduced pressure to afford compound 20s (1.6 g, yield 99%) as a white solid.

N-Boc-tert-leucine 20y (1.5 g, 21.61 mmol, 1.0 eq.) and dichloromethane (70 mL) were charged into a round bottom flask (250 mL). The resulting mixture was treated with HATU (12.32 g, 32.42 mmol, 1.5 eq.). Subsequently, the mixture was stirred at ambient temperature for 15 minutes, and then cooled to 0° C. Diisopropylethylamine (18.8 mL, 108.1 mmol, 5.0 eq.) was added as a single portion followed by compound 20s (3.90 g, 21.61 mmol, 1.0 eq.). The reaction mixture was allowed to warm up to ambient temperature and stirred for 15 hours. The reaction mixture was washed with 5% aqueous citric acid solution (100 mL×2), aqueous sodium hydroxide (1 M, 100 mL×2) and brine, dried over magnesium sulfate, filtered and concentrated in vacuo to give compound 20t (7.8 g, yield 99%) as a dark red oil, which was used in the next step without further purification. MS (ESI) m/z (M+H)⁺ 359.4.

A flask was charged with compound 20t (7.8 g, 21.73 mmol, 1.0 eq.), water (100 mL), and tetrahydrofuran (100 mL). Subsequently, lithium hydroxide monohydrate (2.74 g, 65.19 mmol, 3.0 eq.) was added portion-wise and the mixture was stirred at ambient temperature for 72 hours. The volatile solvents were removed in vacuo. The residue was diluted with aq. NaOH (1 M, 50 mL), and the resulting mixture was extracted with diethyl ether (100 mL×2). The aqueous layer was then acidified with aq.HCl (1 M) to pH=4, and extracted with ethyl acetate (100 mL×3). The combined organic layer was washed with water and brine, dried over magnesium sulfate, filtered and concentrated in vacuo to give compound 20u (6.7 g, yield 90%) as a beige solid, which was used in the next step without further purification. MS (ESI) m/z (M+H)⁺ 345.2. ¹H NMR (400 MHz, DMSO-d₆): δ 12.39 (brs, 1H), 6.51 (d, J=9.2 Hz, 1H), 5.19 (brs, 1H), 4.32 (brs, 1H), 4.28 (d, J=8.4 Hz, 1H), 4.16 (d, J=9.2 Hz, 1H) 3.67-3.58 (m, 2H), 2.15-2.09 (m, 1H), 1.92-1.85 (m, 1H), 1.39 (s, 9H), 0.93 (s, 9H).

A flask was charged with compound 20u (584 mg, 1.7 mmol, 1.0 eq.) and N,N-dimethylformamide (10 mL). HATU (969 mg, 2.6 mmol, 1.5 eq.) was added as a single portion and the reaction mixture stirred at ambient temperature for 30 minutes, and then cooled to 0° C. Diisopropylethylamine (2.5 mL, 17 mmol, 10.0 eq.) was added as a single portion followed by compound 20q (520 mg, 1.7 mmol, 1.0 eq.). The mixture was allowed to warm up to ambient temperature and stirred for 12 hrs and the mixture was then diluted with water (40 mL) and acidified to pH=3-4 with citric acid (aq.). The aqueous layer was extracted with ethyl acetate (30 mL×3). The combined organic layer was washed with brine, dried over sodium sulfate, filtered and the solvent was removed in vacuo. The residue was purified by Prep-HPLC to provide compound 20v as a white solid (440 mg, yield 41%). MS (ESI) m/z (M+H)⁺ 627.3.

Compound 20w (194 mg, 0.31 mmol, 1.0 eq.) was dissolved in dimethylsulfoxide (2 mL). To the solution was added potassium tert-Butoxide (138 mg, 1.24 mmol, 4.0 eq.) in portions. The mixture was stirred at ambient temperature for 15 minutes. Compound 20i (120 mg, 0.31 mmol, 1.1 eq.) was added into the mixture. Stirring was continued for additional 4 hours, then the mixture was diluted with water (5 mL) and acidified to pH=3˜4 with citric acid (aq.). The aqueous layer was extracted with ethyl acetate (30 mL×3). The combined organic layer was washed with brine, dried over sodium sulfate, filtered and the solvent was removed in vacuo. The residue was purified by flash column chromatography (petroleum ether/EtOAc=1/1) to afford compound 20x (160 mg, yield 53%) as a pale yellow solid. MS (ESI) m/z (M+H)⁺ 978.8.

A flask was charged with compound 20x (164 mg, 0.18 mmol, 1.0 eq.), Hoveyda-Grubbs catalyst (10.5 mg, 0.018 mmol, 10 mol %.) and anhydrous DCE (300 mL). The reaction mixture was heated to reflux overnight. And then the reaction mixture was allowed to cool down to ambient temperature. The solvent was removed in vacuo, the resulting residue was purified by prep-TLC (petroleum ether/EtOAc=1/1) to provide compound 101 (150 mg, yield 94%) as a white solid. MS (ESI) m/z (M+H)⁺ 950.7.

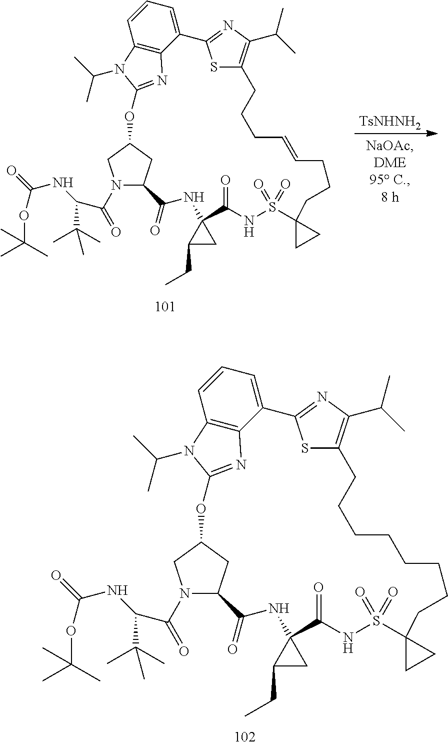

Compound 101 (110 mg, 0.12 mmol, 1.0 eq.) was dissolved in DME (0.9 mL). To this solution was added H₂O (0.1 mL), p-TosNHNH₂ (162 mg, 0.87 mmol, 7.5 eq.) and NaOAc (144 mg, 1.74 mmol, 15.0 eq.). The suspension was stirred at reflux for 8 hrs. Then the mixture was cooled to room temperature, diluted with EtOAc (100 mL), washed with aq. NaHCO₃ and aq. citric acid, followed by brine. The organic layer was dried over sodium sulfate, and filtered to remove the solid. The solvent was then removed in vacuo to afford a residue. The residue was purified by prep-HPLC to afford compound 102 (60 mg, yield 54%) as a white solid. MS (ESI) m/z (M+H)⁺ 952.4.

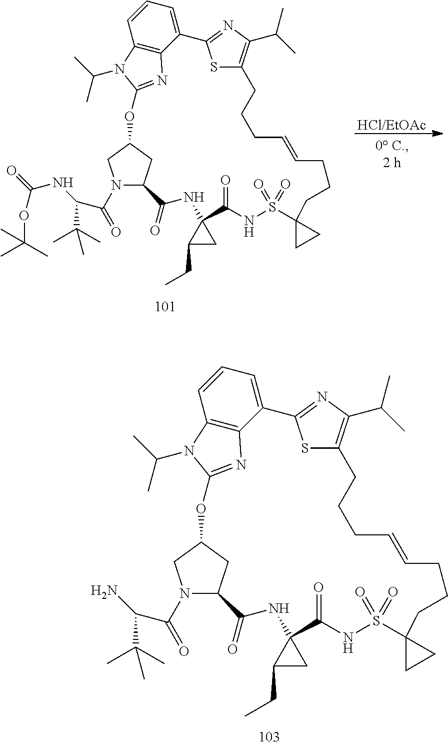

Compound 101 (10 mg, 0.01 mmol) was dissolved in a solution of HCl (g) in EtOAc (4 M, 0.5 mL), the resulting solution was stirred at 0° C. The reaction was monitored by LCMS. The solution was concentrated in vacuo to afford compound 103 as HCl salt (7.5 mg, yield 81%). MS (ESI) m/z (M+H)⁺ 850.5.

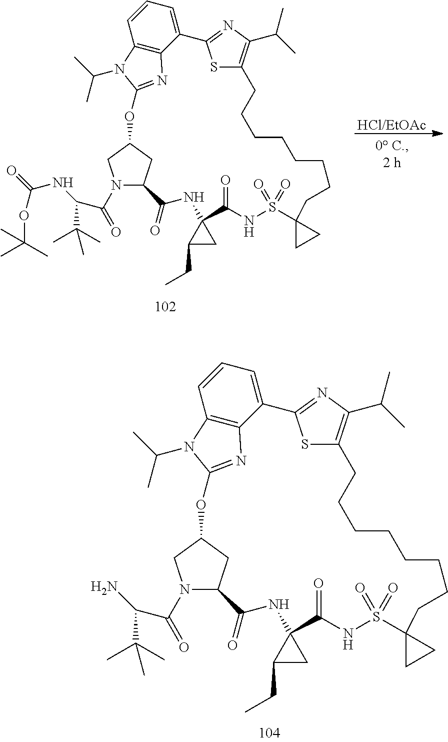

Compound 104 was prepared from compound 102 according to the procedure for the preparation of compound 103. (9.1 mg, yield 98%). MS (ESI) m/z (M+H)⁺ 852.3.

### Preparation of Compounds 105-110

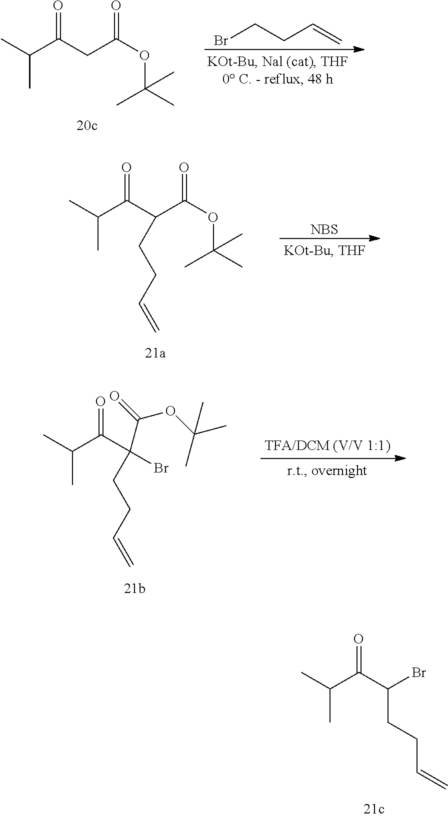

To a solution of compound 20c (20 g, 0.11 mol) in THF (300 mL) was added KOt-Bu (14.6 g, 0.13 mol) in portions at 0° C. After 1 h, NaI (8.1 g, 0.08 mol) and 4-bromobut-1-ene (17.3 g, 0.13 mol) was added. The mixture was heated to reflux for 48 hrs. After being cooled to r.t., the reaction was quenched with ice-water (40 mL), extracted with EtOAc (100 mL×3). The combined organic layer was washed with water and brine, dried over Na₂SO₄ and concentrated in vacuo. The residue was purified by column chromatography on silica gel (petroleum ther/EtOAc=100/1) to give compound 21a (18 g, yield 69%) as a light yellow oil. MS (ESI) m/z (M+Na)⁺262.9. ¹H NMR (400 MHz, CDCl₃): δ 5.79-5.71 (m, 1H), 5.05-4.96 (m, 2H), 3.53 (t, J=7.2 Hz, 1H), 2.82-2.75 (m, 1H), 2.06-2.00 (m, 2H), 1.92-1.85 (m, 2H), 1.44 (s, 9H), 1.09 (dd, J=7.2, 7.2 Hz, 6H).

To a solution of compound 21a (18 g, 76 mmol) in THF (150 mL) was added KOt-Bu (10.3 g, 92 mmol) in portions at 0° C. After 30 min, NBS (16.2 g, 92 mmol) was added in one portion. The mixture was allowed to warm to r.t. and stirred for 12 hrs. The reaction mixture was diluted with EtOAc (200 mL), the organic layer was washed with water and brine, then dried over Na₂SO₄. The solution was removed in vacuo and the residue was distilled to give compound 21b (18.5 g, yield 76%). ¹H NMR (400 MHz, CDCl₃): δ 5.87-5.75 (m, 1H), 5.07 (d, J=17.2 Hz, 1H), 5.00 (d, J=10 Hz, 1H), 3.12-3.06 (m, 1H), 2.28-2.24 (m, 2H), 2.23-2.05 (m, 2H), 1.49 (s, 9H), 1.25 (d, J=6.8 Hz, 3H), 1.16 (d, J=6.8 Hz, 3H).

To a solution of compound 21b (18 g, 56.6 mmol) in DCM (40 mL) was added TFA (60 mL) dropwise at 0° C. After addition, the resulting solution was heated at 40° C. for 24 hrs. The solution was concentrated in vacuo below 35° C. The residue was diluted with EtOAc (100 mL), the organic layer was washed with saturated aq. NaHCO₃ and brine, dried over Na₂SO₄ and concentrated in vacuo. The residue was distilled to provide compound 21c (6 g, yield 48%). ¹H NMR (400 MHz, CDCl₃): δ 5.79-5.70 (m, 1H), 5.07 (d, J=17.2 Hz, 1H), 5.03 (d, J=10.4 Hz, 1H), 4.43-4.38 (m, 1H), 3.07-3.00 (m, 1H), 2.20-1.98 (m, 4H), 1.17 (d, J=6.8 Hz, 3H), 1.12 (d, J=6.8 Hz, 3H).

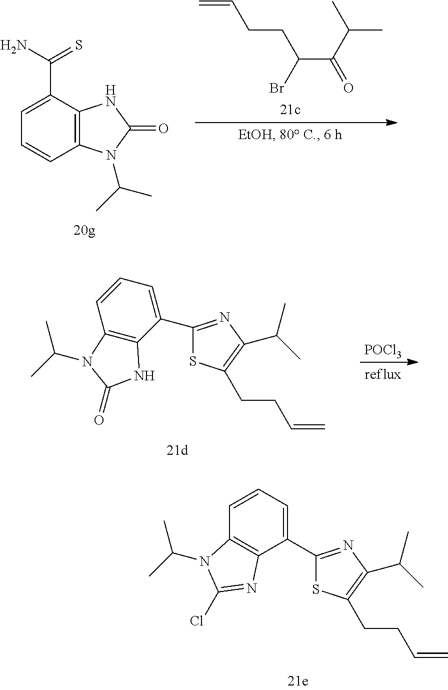

A mixture of compound 20g (1 g, 4.3 mmol) and compound 21c (1 g, 4.6 mmol) in EtOH (10 mL) was refluxed for 6 hrs. And then the reaction mixture was concentrated in vacuo, the residue was diluted with EtOAc (60 mL) and washed with brine. The organic layer was separated, dried over Na₂SO₄ and concentrated in vacuo. The residue was purified by prep-TLC (PE/EtOAc=3/1) to give compound 21d (150 mg, yield 10%) as a light yellow solid. MS (ESI) m/z (M+H)⁺ 356.3. ¹H NMR (300 MHz, CDCl₃): δ 9.74 (brs, 1H), 7.31 (d, J=6.9 Hz, 1H), 7.13-7.02 (m, 2H), 5.90-5.79 (m, 1H), 5.07 (d, J=17.2 Hz, 1H), 5.03 (d, J=8.4 Hz, 1H), 4.79-4.70 (m, 1H), 3.13-3.05 (m, 1H), 2.93-2.85 (m, 2H), 2.43-2.45 (m, 2H), 1.54 (d, J=6.9 Hz, 6H), 1.32 (d, J=6.9 Hz, 6H).

Compound 21d (340 mg, 0.96 mmol) was dissolved in POCl₃ (1 mL). The mixture was heated to reflux for 12 hrs. After being cooled to r.t., it was concentrated; the resulting residue was diluted with EtOAc (60 mL) and neutralized with saturated aq. NaHCO₃. The organic layer was washed with brine, dried over Na₂SO₄ and concentrated in vacuo to give a brown residue. The residue was purified by prep-TLC (PE/EtOAc=3/1) to provide compound 21e as a light yellow solid (330 mg, yield 93%). MS (ESI) m/z (M+H)⁺ 373.9.

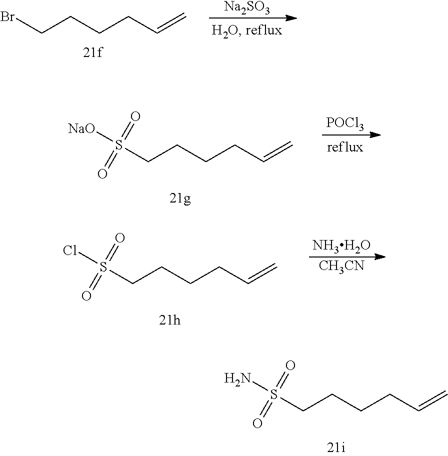

To a solution of sodium sulphite (8.86 g, 70.3 mmol) in water (30 mL) was added compound 21f (9.5 g, 58.6 mmol). The mixture was heated to reflux overnight. After cooling to r.t., water was removed under reduced pressure. The resulting solid was further dried under vacuum to give crude compound 21g as a white solid (18 g, yield 100%).

¹-H NMR (400 MHz, D₂O) δ 5.83-5.73 (m, 1H), 5.02-4.87 (m, 2H), 2.82-2.75 (m., 2H), 2.13-1.95 (m, 2H), 1.66-1.59 (m, 2H), 1.45-1.35 (m, 2H).

A flask was charged with compound 21g (9 g, 29.3 mmol) and phosphorus oxychloride (50 g). The mixture was heated to reflux for 6 hrs. After cooling to r.t., the mixture was diluted with CH₂Cl₂ (50 mL), and filtered. The filtrate was concentrated to remove the solvent and excess phosphorus oxychloride. The dark oil was diluted with CH₂Cl₂ (50 mL), and washed with water (50 mL) and brine (50 mL). The organic layer was dried over Na₂SO₄, concentrated under vacuum to provide compound 21h as dark brown oil (4 g, yield 75%).

A solution of compound 21h (4 g, 21.9 mmol) in acetonitrile (50 mL) was added dropwise at 0° C. to aq. ammonia (50 mL, 25%). After addition, the mixture was stirred at same temperature for additional 1 hour. The mixture was diluted with 100 mL of brine, and the aqueous layer was extracted EtOAc (50 mL×3). The combined organic layer was dried over Na₂SO₄, concentrated under vacuum to afford compound 21i as a brown solid (3 g, yield 83%). ¹-H NMR (300 MHz, CDCl₃): δ 5.84-5.70 (m, 1H), 5.07-4.97 (m, 2H), 4.64 (brs, 2H), 3.17-3.09 (m, 2H), 2.16-2.08 (m, 2H), 1.92-1.82 (m, 2H), 1.62-1.50 (m, 2H).

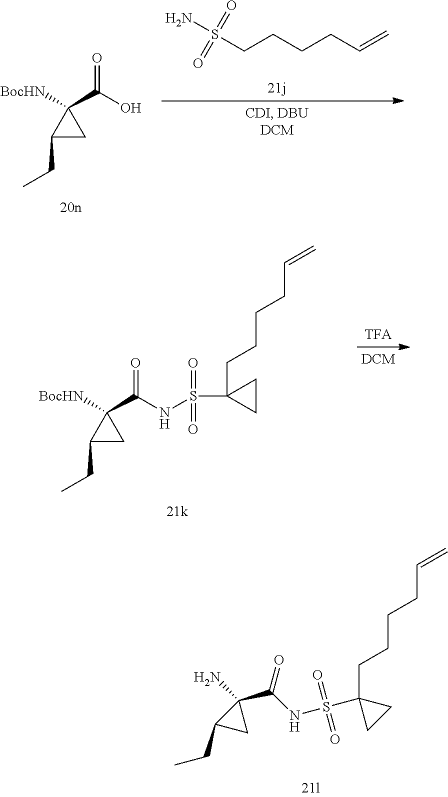

A flask was charged with compound 20n (800 mg, 3.5 mmol, 1.0 eq.) and dichloromethane (10 mL). 1,1′-Carbonyldiimidazole (850 mg, 5.3 mmol, 1.5 eq.) was added as a single portion and the mixture stirred under reflux for 2 hrs. Then the resulting mixture was cooled to r.t. To the mixture was added compound 21j (1100 mg, 7.0 mmol, 2.5 eq.) and DBU (1600 mg, 10.5 mmol, 3 eq.). The mixture was heated to reflux and stirring was continued for additional 8 hrs. The mixture was concentrated and diluted with water (20 mL), the aqueous layer was adjusted to pH=4-5 with citric acid (aq.) and extracted with EtOAc (50 mL×3). The combined organic layer was washed with brine, dried over sodium sulfate, filtered and the solvent was removed in vacuo. The residue was purified by prep-TLC (PE/EtOAc=1/3) to give compound 21k (679 mg, yield 52%) as a white solid. MS (ESI) m/z (M+H)⁺ 415.2.

Compound 21k (860 mg, 2.3 mmol) was dissolved in dichloromethane (2 mL). TFA (1 mL) was added dropwise and the mixture was stirred at r.t. for 2 hrs. The solvent was concentrated in vacuo, the residue was diluted with EtOAc (50 mL) and neutralized with saturated aq. NaHCO₃. Then a white solid precipitated in the organic layer and was filtered, the solid compound 211 was the desired product, which was detected by LCMS (530 mg, yield 84%). MS (ESI) m/z (M+H)⁺ 315.3.

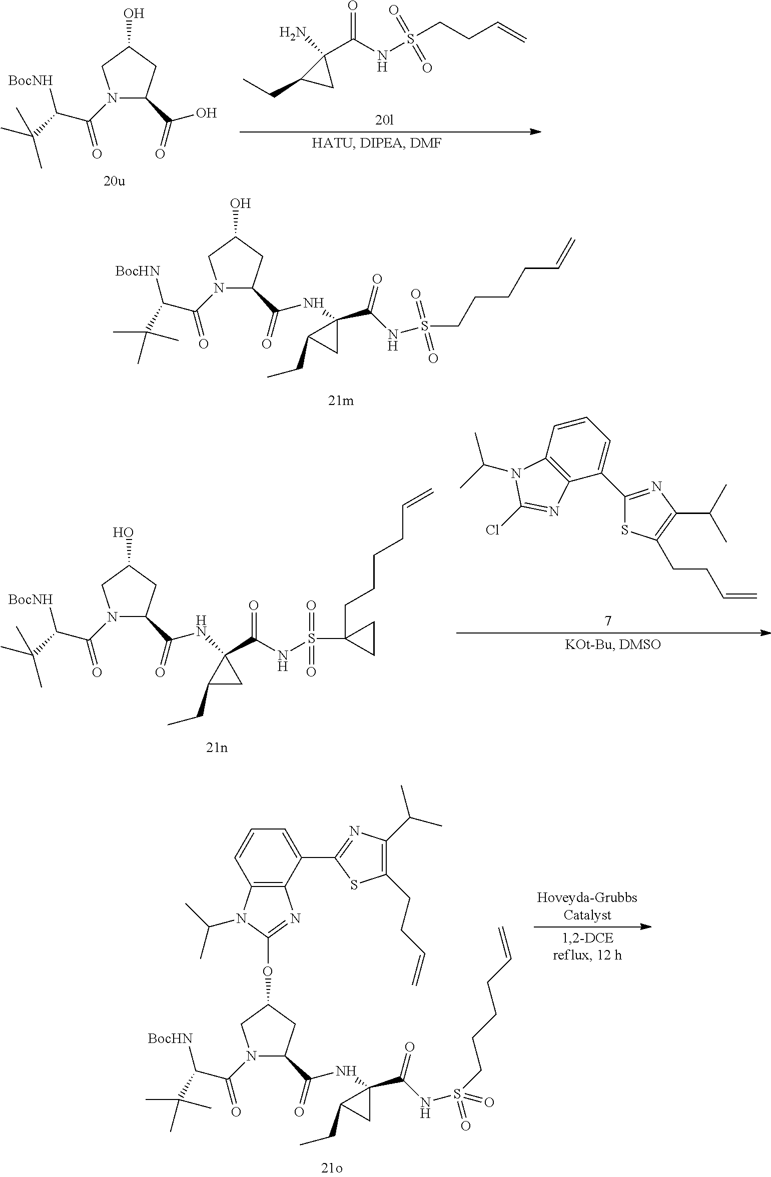

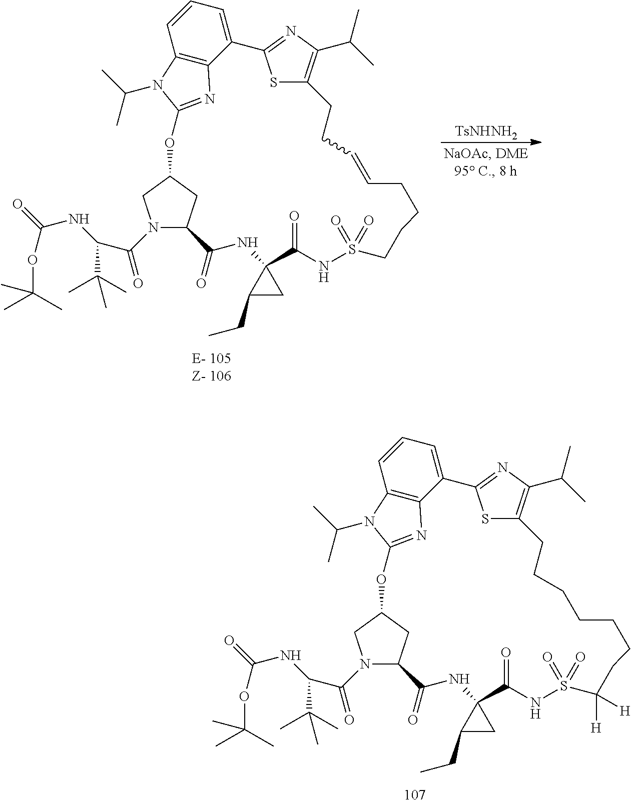

A flask was charged with compound 20u (653 mg, 1.9 mmol, 1.0 eq.) and N,N-dimethylformamide (12 mL). HATU (1140 mg, 3 mmol, 1.5 eq.) was added as a single portion and the mixture was stirred at ambient temperature for 30 minutes then cooled to 0° C. Diisopropylethylamine (3.0 mL, 20 mmol, 10.0 eq.) was added as a single portion followed by compound 201 (520 mg, 1.9 mmol, 1.0 eq.). The mixture was allowed to warm up to ambient temperature and stirring was continued for another 12 hrs. The mixture was diluted with water (40 mL) and acidified to pH=3-4 with citric acid (aq.). The aqueous layer was extracted with ethyl acetate (30 mL×3). The combined organic layer was washed with brine, dried over sodium sulfate, filtered and concentrated in vacuo. The residue was purified by Prep-HPLC to give compound 21m as a white solid (480 mg, yield 42%). MS (ESI) m/z (M+Na)⁺ 623.2.

Compound 210 (420 mg, yield 58%) was obtained as a light yellow solid from 460 mg of compound 21n using the same procedure as that for the preparation of compound 20x. MS (ESI) m/z (M+H)⁺ 938.5.

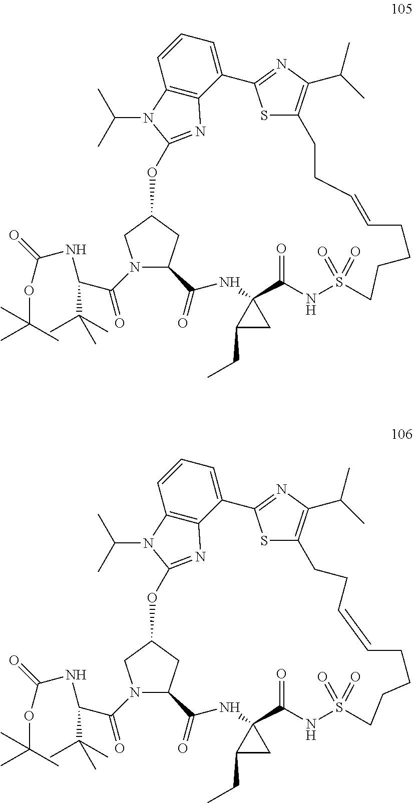

Compounds 105 and 106 were prepared using a similar procedure as that for the preparation of compound 101. The two isomers were isolated by Prep-HPLC, 120 mg of each isomer (yield 62% totally) was obtained as a light yellow solid from 400 mg of compound 21o. MS (ESI) m/z (M+H)⁺ 910.5.

Compound 107 (54 mg, yield 54%) was obtained using the same procedure for the preparation of compound 102 as a light yellow solid from 100 mg of mixture of compounds 105 & 106. MS (ESI) m/z (M+H)⁺ 912.4.

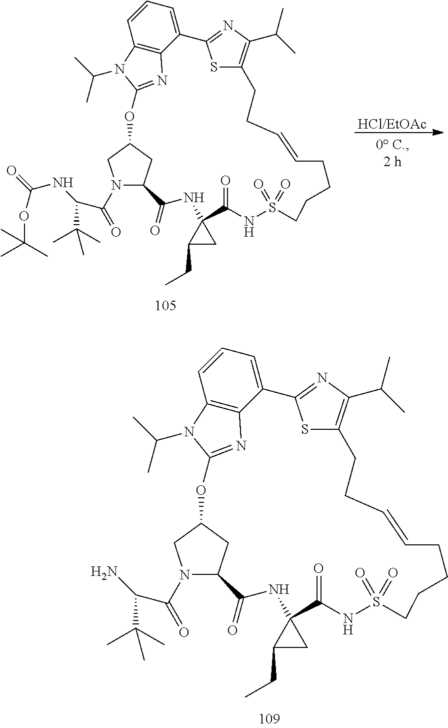

Compound 109 was prepared using the same procedure as that for the preparation of 103. (7.6 mg, yield 58%). MS (ESI) m/z (M+H)⁺ 810.4.

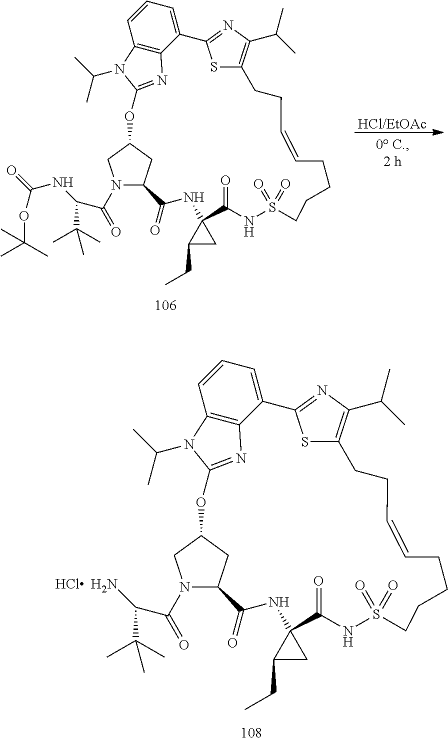

Compound 108 was prepared using the same procedure as that for the preparation of compound 103. (7.7 mg, yield 59%). MS (ESI) m/z (M+H)⁺ 810.4.

Compound 110 was prepared using the same procedure as that for the preparation of compound 103. (14.6 mg, yield 98%). MS (ESI) m/z (M+H)⁺ 812.4.

### Preparation of Compounds 111-113

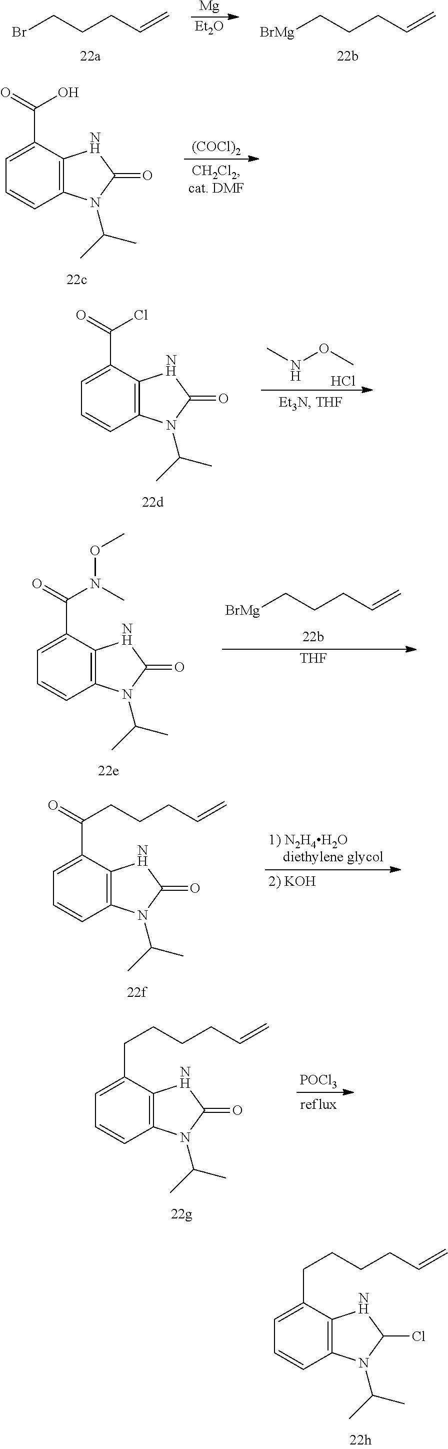

A round-bottomed flask (250-mL, three-necked) was charged with magnesium turnings (2.34 g, 97.5 mmol) and 10 mL of anhydrous Et₂O. The flask was purged with nitrogen. Then a solution of 5-bromo-1-pentene 22a (7.9 g, 48.7 mmol) in anhydrous Et₂O (60 mL) was added using a dropping funnel at a rate adjusted to maintain a gentle reflux. The resulting mixture was then stirred for an additional hour to give compound 22b (solution in Et₂O).

To a suspension of compound 22c (2 g, 9.05 mmol) in CH₂Cl₂ (20 mL) was added oxalyl chloride (1.72 g, 13.57 mmol) and two drops of DMF at r.t. The mixture was stirred at r.t. for 3 hrs. The solvent and excess oxalyl chloride was removed under vacuum to give crude acid chloride 22d as yellow oil (2.2 g, yield 100%).

The crude acid chloride 22d (2.2 g, 9.05 mmol) was dissolved in dry THF. To the solution was added N,O-dimethylhydroxylamine HCl salt (1.76 g, 18.1 mmol) and Et₃N (3.65 g, 36.2 mmol). The mixture was stirred at r.t. overnight. The solvent was removed under reduced pressure. The residue was diluted with water (50 mL), extracted with EtOAc (50 mL×3). The combined organic layer was dried over Na₂SO₄, concentrated in vacuo. The residue was purified by column chromatography on silica gel (PE/EA=3/1) to provide compound 22e (1.6 g, yield 67%) as a white solid. ¹H NMR (400 MHz, CDCl₃): δ 9.17 (brs, 1H), 7.59 (d, J=8 Hz, 1H), 7.20 (d, J=8.0 Hz, 1H), 7.03 (t, J=8.0 Hz, 1H), 4.75-4.69 (m, 1H), 3.63 (s, 3H), 3.40 (s, 3H), 1.53 (d, J=6.8 Hz, 6H).

Compound 22e (1.28 g, 4.87 mmol) was dissolved in anhydrous THF (10 mL). To the solution was added dropwise a solution of compound 22b in Et₂O (0.7 M, 70 mL, 49 mmol) at 0° C. The mixture was allowed to warm to r.t. and stirred overnight. The mixture was treated with aq. HCl (1M) and the aqueous layer was adjusted to pH=7-8. The aqueous layer was extracted with EtOAc (30 mL×3). The combined organic layer was washed with brine, dried over Na₂SO₄, and concentrated in vacuo. The residue was purified by column chromatography on silica gel (PE/EA=5/1) to afford compound 22f as a colorless oil (0.85 g, yield 64%). ¹H NMR (300 MHz, CDCl₃): δ 9.58 (brs, 1H), 7.52 (d, J=8.1 Hz, 1H), 7.28 (d, J=8.1 Hz, 1H), 7.08 (t, J=8.1 Hz, 1H), 5.87-5.75 (m, 1H), 5.04 (d, J=16.8 Hz, 1H), 5.01 (d, J=9.9 Hz, 1H), 4.78-4.68 (m, 1H), 3.03-2.96 (m, 2H), 2.21-2.12 (m, 2H), 1.90-1.80 (m, 2H), 1.52 (d, J=6.9 Hz, 6H).

A flask was charged with compound 22f (0.1 g, 0.367 mmol), diethylene glycol (2 mL), N₂H₄.H₂O (65 mg, 1.1 mmol, 85% in water) and EtOH (2 mL). The mixture was heated at 100° C. for 2 hrs under nitrogen atmosphere. The mixture was cooled to room temperature, and then KOH (41 mg, 0.734 mmol) was added. The mixture was stirred at r.t. for 15 min. Water and EtOH were removed under reduced pressure. The residue was heated at 200° C. for 1.5 hrs. The mixture was diluted with 10 mL of water, and the aqueous layer was extracted with EtOAc (20 mL×3). The combined organic layer was dried over Na₂SO₄, concentrated in vacuo. The residue was purified by prep-TLC (PE/EA=6/1) to afford compound 22g as colorless oil (78 mg, yield 78%). ¹H NMR (400 MHz, CDCl₃): δ 10.79 (brs, 1H), 7.02-6.97 (m, 2H), 6.90-6.85 (m, 1H), 5.88-5.78 (m, 1H), 5.02 (d, J=16.8 Hz, 1H), 4.94 (d, J=10.4 Hz, 1H), 4.77-4.68 (m, 1H), 2.77-2.72 (m, 2H), 2.17-2.10 (m, 2H), 1.75-1.67 (m, 2H), 1.59-1.51 (m, 2H), 1.56 (d, J=7.2 Hz, 6H).

A mixture of compound 22g (90 mg, 0.349 mmol) and phosphorus oxychloride (2 mL) was heated to reflux for 2 hrs. The excess phosphorus oxychloride was removed under reduced pressure. The residue was dissolved in EtOAc (50 mL), and the organic layer was washed with saturated aq. NaHCO₃ and brine successively, dried over Na₂SO₄, concentrated in vacuo. The residue was purified by prep-TLC (PE/EA=5/1) to afford compound 22h (80 mg, yield 83%) as colorless oil. ¹H NMR (400 MHz, CDCl₃): δ 7.33 (d, J=8.0 Hz, 1H), 7.17 (t, J=8.0 Hz, 1H), 7.06 (d, J=8.0 Hz, 1H), 5.88-5.76 (m, 1H), 5.04-4.88 (m, 3H), 3.05-2.99 (m, 2H), 2.13-2.07 (m, 2H), 1.83-1.74 (m, 2H), 1.65 (d, J=7.2 Hz, 6H), 1.55-1.47 (m. 2H).

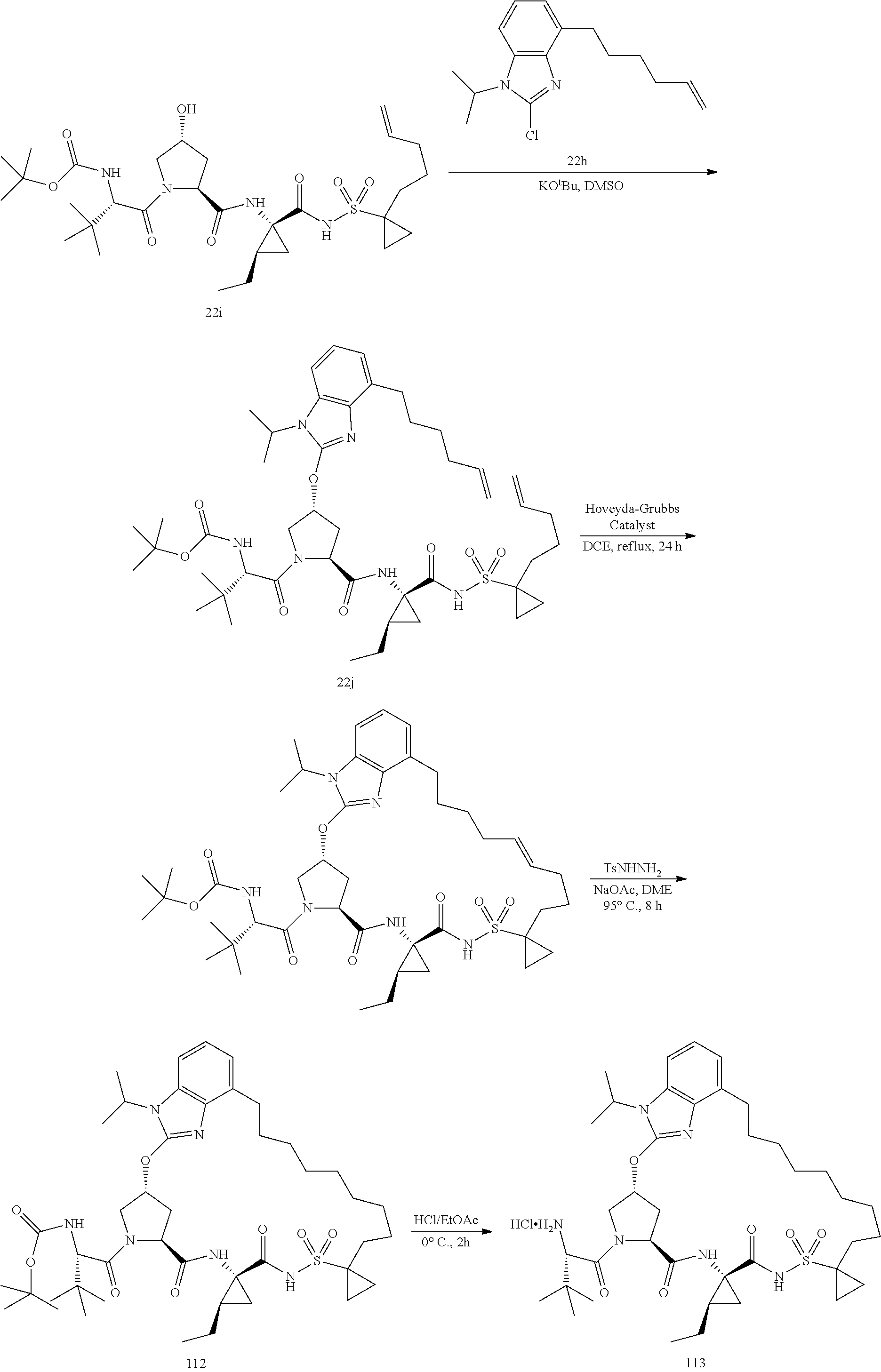

A flask was charged with compound 22i (200 mg, 0.32 mmol, 1.0 eq.) and dimethylsulfoxide (1 mL). Potassium tert-Butoxide (143 mg, 1.28 mmol, 4.0 eq.) was added portion-wise to the flask. The mixture was stirred at ambient temperature for 5 minutes. Compound 22h (88 mg, 0.32 mmol, 1.0 eq.) was dissolved in dimethylsulfoxide (1 mL) and the resulting solution was added dropwise to the mixture in the flask. Stirring was continued for 24 hours. LCMS indicated there was still starting material 22i existed. The mixture was treated with water and acidified to pH 3-4 with citric acid (aq.). The aqueous layer was extracted with ethyl acetate (30 mL×3). The organic layers were combined, washed with brine, dried over magnesium sulfate, filtered and the solvent was removed in vacuo. The residue was purified by prep-TLC (PE/EA=3/1) to provide compound 22j as pale yellow solid (60 mg, yield 55%) and 120 mg of compound 22i. MS (ESI) m/z (M+H)⁺ 867.6.

Compound 111 was prepared using the procedure as that for the preparation of compound 101. (47 mg, yield 49%). MS (ESI) m/z (M+H)⁺ 839.5.

Compound 112 was prepared using the procedure as that for the preparation of compound 102. (70 mg, yield 70%). MS (ESI) m/z (M+H)⁺ 841.5.

Compound 113 was prepared using the procedure as that for the preparation of compound 103 (15 mg, yield 83%). MS (ESI) m/z (M+H)⁺ 741.4.

### Example A

### NS3-NS4 Protease Assay

NS3 Complex Formation with NS4A-2.

Recombinant E. coli or Baculovirus full-length NS3 was diluted to 3.33 μM with assay buffer and the material was transferred to an eppendorf tube and placed in a water bath in a 4° C. refrigerator. The appropriate amount of NS4A-2 diluted to 8.3 mM in assay buffer was added to an equal the volume of NS3 above (conversion factor—3.8 mg/272 μL assay buffer). The material was transferred to an eppendorf tube and placed in water bath in a 4° C. refrigerator.

After equilibration to 4° C., equal volumes of NS3 and NS4A-2 solutions were combined in an eppendorf tube, mixed gently with a manual pipettor, and incubated for 15 minutes in the 4° C. water bath. Final concentrations in the mixture are 1.67 μM NS3, 4.15 mM NS4A-2 (2485-fold molar excess NS4A-2).

After 15 minutes at 4° C., the NS3/NS4A-2 eppendorf tube was removed and placed in a room temperature water bath for 10 minutes. NS3/NS4A-2 was aliquoted at appropriate volumes and stored at −80° C. (E. coli NS3 run at 2 nM in assay, aliquot at 25 μL. BV NS3 run at 3 nM in assay, aliquot at 30 μL).

### Example B

### NS3 Inhibition Assay

Step a. Sample compounds were dissolved to 10 mM in DMSO then diluted to 2.5 mM (1:4) in DMSO. Typically, compounds were added to an assay plate at 2.5 mM concentration, yielding upon dilution a starting concentration of 50 μM in the assay inhibition curve. Compounds were serial diluted in assay buffer to provide test solutions at lower concentrations.

Step 1. The E. coli. NS3/NS4A-2 was diluted to 4 nM NS3 (1:417.5 of 1.67 μM stock−18 μL 1.67 μM stock+7497 μL assay buffer). The BV NS3/NS4A-2 was diluted to 6 nM NS3 (1:278.3 of 1.67 μM stock−24 μL 1.67 μM stock+6655 μL assay buffer).

Step. 2. Using the manual multichannel pipettor, and being careful not to introduce bubbles into the plate, 50 μL assay buffer was added to wells A01-H01 of a black Costar 96-well polypropylene storage plate.

Step 3. Using the manual multichannel pipettor, and being careful not to introduce bubbles into the plate, 50 μL of diluted NS3/NS4A-2 from step 1 was added to wells A02-H12 of the plate in step 2.

Step 4. Using the manual multichannel pipettor, and being careful not to introduce bubbles into the plate, 25 μL of the wells in drug dilution plate in step a was transferred to corresponding wells in assay plate in step 3. The tips on the multichannel pipettor were changed for each row of compounds transferred.

Step 5. Using the manual multichannel pipettor, and being careful not to introduce bubbles into the plate, the contents of the wells from the assay plate in step 4 were mixed by aspirating and dispensing 35 μL of the 75 μL in each well five times. The tips on multichannel pipettor were changed for each row of wells mixed.

Step 6. The plate was covered with a polystyrene plate lid, and the plate from step 5 containing NS3 protease and sample compounds was pre-incubated 10 minutes at room temperature.

While the plate from step 6 is pre-incubating, the RETS1 substrate was diluted in a 15 mL polypropylene centrifuge tube. The RETS1 substrate was diluted to 8 μM (1:80.75 of 646 μM stock−65 μL 646 μM stock+5184 μL assay buffer).

After the plate in step 6 finished pre-incubating, and using the manual multichannel, 25 μL of substrate was added to all wells on the plate. The contents of the wells of the plate were quickly mixed, as in step 5, mixing 65 μL of the 100 μL in the wells.

The plate was read in kinetic mode on the Molecular Devices SpectraMax Gemini XS plate reader. Reader settings: Read time: 30 minutes, Interval: 36 seconds, Reads: 51, Excitation λ: 335 nm, Emission λ: 495 nm, cutoff: 475 nm, Automix: off, Calibrate: once, PMT: high, Reads/well: 6, Vmax pts: 21 or 28/51 depending on length of linearity of reaction

IC₅₀s are determined using a four parameter curve fit equation, and converted to Ki's using the following Km's:
Full-length E. coli NS3-2.03 μM
Full-length BV NS3-1.74 μM
where Ki=IC₅₀/(1+[S]/Km))

### Quantitation by ELISA of the Selectable Marker Protein, Neomycin Phosphotransferase II (NPTII) in the HCV Sub-Genomic Replicon, GS4.3

The HCV sub-genomic replicon (I377/NS3-3′, accession No. AJ242652), stably maintained in HuH-7 hepatoma cells, was created by Lohmann et al. Science 285: 110-113 (1999). The replicon-containing cell culture, designated GS4.3, was obtained from Dr. Christoph Seeger of the Institute for Cancer Research, Fox Chase Cancer Center, Philadelphia, Pa.

GS4.3 cells were maintained at 37° C., 5% CO₂, in DMEM (Gibco 11965-092) supplemented with L-glutamine 200 mM (100×) (Gibco25030-081), non-essential amino acids (NEAA)(Biowhittaker 13-114E), heat-inactivated (HI) Fetal Bovine Serum(FBS)(Hyclone SH3007.03) and 750 geneticin (G418)(Gibco 10131-035). Cells were sub-divided 1:3 or 4 every 2-3 days.

24 h prior to the assay, GS4.3 cells were collected, counted, and plated in 96-well plates (Costar 3585) at 7500 cells/well in 100 μL standard maintenance medium (above) and incubated in the conditions above. To initiate the assay, culture medium was removed, cells were washed once with PBS (Gibco 10010-023) and 90 μl Assay Medium (DMEM, L-glutamine, NEAA, 10% HI FBS, no G418) was added. Inhibitors were made as a 10× stock in Assay Medium, (3-fold dilutions from 10 μM to 56 μM final concentration, final DMSO concentration 1%), 10 μL were added to duplicate wells, plates were rocked to mix, and incubated as above for 72 h.

An NPTII Elisa kit was obtained from AGDIA, Inc. (Compound direct ELISA test system for Neomycin Phosphotransferase II, PSP 73000/4800). Manufacturer's instructions were followed, with some modifications. 10×PEB-1 lysis buffer was made up to include 500 μM PMSF (Sigma P7626, 50 mM stock in isopropanol). After 72 h incubation, cells were washed once with PBS and 150 μL PEB-1 with PMSF was added per well. Plates were agitated vigorously for 15 minutes, room temperature, and then frozen at −70° C. Plates were thawed, lysates were mixed thoroughly, and 100 μL were applied to an NPTII Elisa plate. A standard curve was made. Lysate from DMSO-treated control cells was pooled, serially diluted with PEB-1 with PMSF, and applied to duplicate wells of the ELISA plate, in a range of initial lysate amount of 150 μL. In addition, 100 μL buffer alone was applied in duplicate as a blank. Plates were sealed and gently agitated at room temperature for 2 h. Following capture incubation, the plates were washed 5×300 μL with PBS-T (0.5% Tween-20, PBS-T was supplied in the ELISA kit). For detection, a 1× dilution of enzyme conjugate diluent MRS-2 (5×) was made in PBS-T, into which 1:100 dilutions of enzyme conjugates A and B were added, as per instructions. Plates were resealed, and incubated with agitation, covered, room temperature, for 2 h. The washing was then repeated and 100 μL of room temperature TMB substrate was added. After approximately 30 minutes incubation (room temperature, agitation, covered), the reaction was stopped with 50 μL 3M sulfuric acid. Plates were read at 450 nm on a Molecular Devices Versamax plate reader.

Inhibitor effect was expressed as a percentage of DMSO-treated control signal, and inhibition curves were calculated using a 4-parameter equation: y=A+4B−A)/(1+((C/x)̂D))), where C is half-maximal activity or EC₅₀.

### Examples of Activity:

Table 1 below provides examples of active compounds.

TABLE 1

| Compound | Structure | EC₅₀ (nM) | IC₅₀ (nM) |
|---|---|---|---|
| 7 | 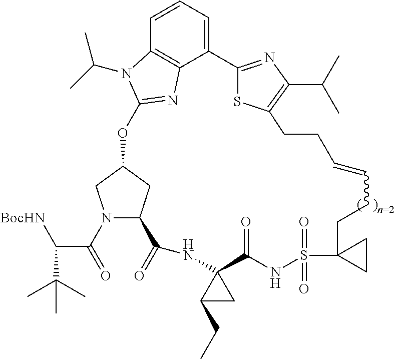 | A | C |
| 8 | 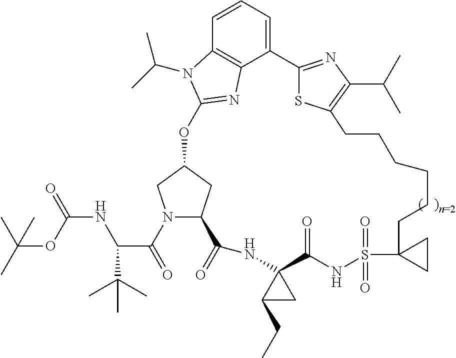 | A | C |
| 9 | 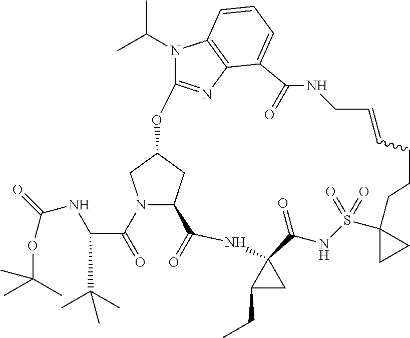 | B | C |
| 10 | 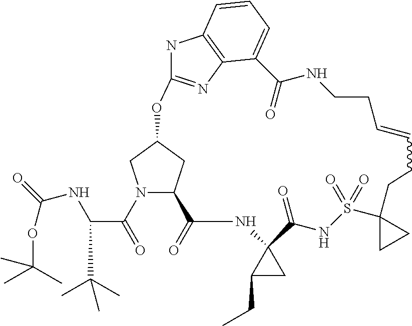 | A | C |
| 11 | 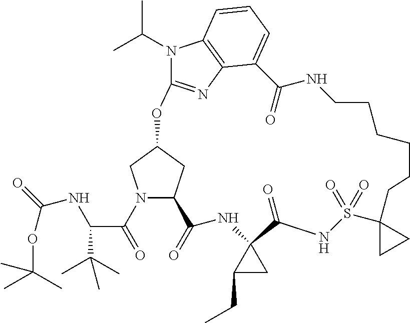 | A | C |
| 12 | 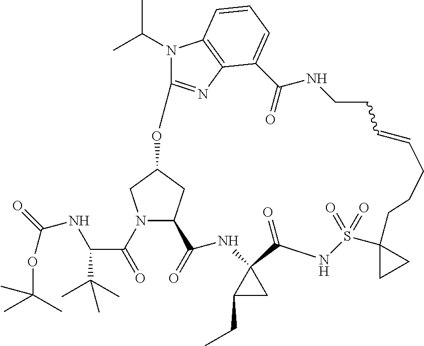 | B | C |
| 13 | 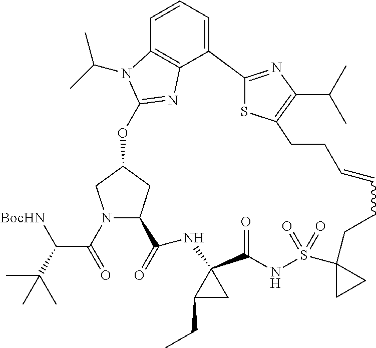 | A | C |
| 14 | 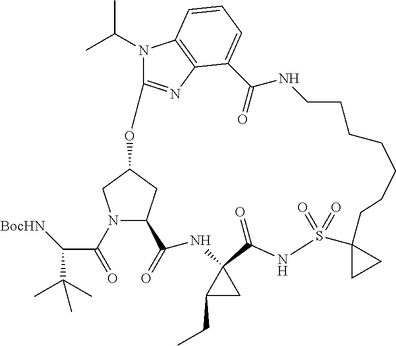 | C | C |
| 15 | 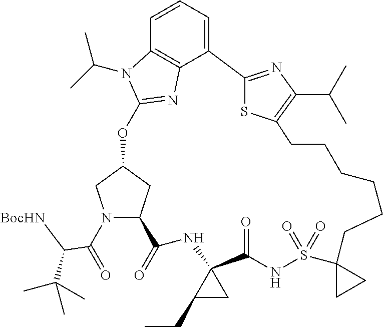 | A | C |
| 101 | 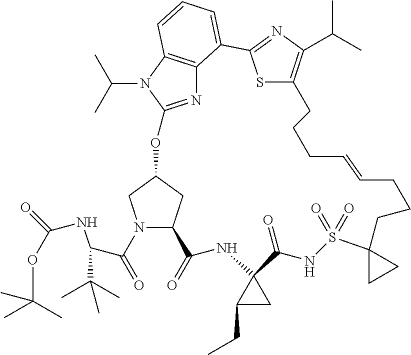 | A | C |
| 102 | 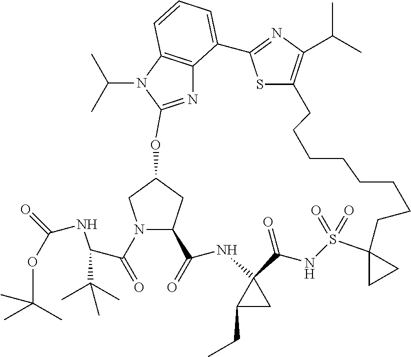 | A | B |
| 103 | 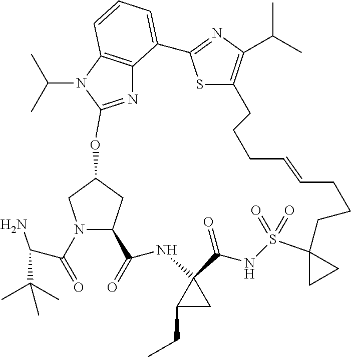 | A | B |
| 104 | 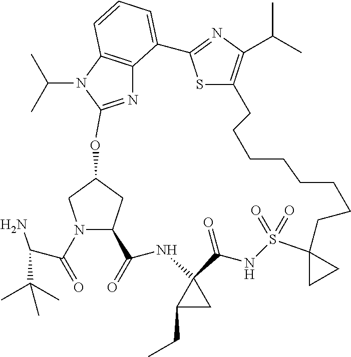 | A | B |
| 105 | 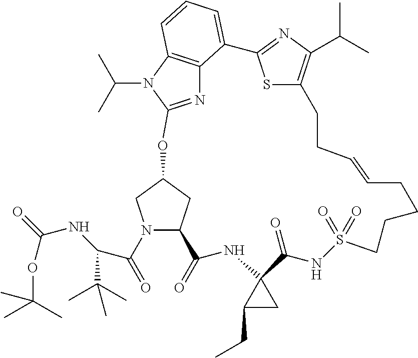 | A | C |
| 106 | 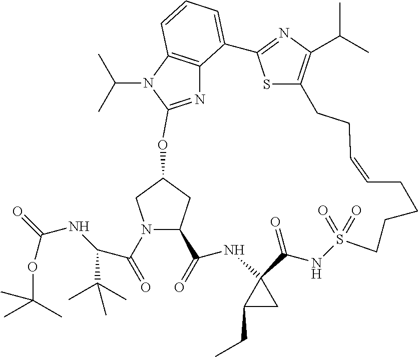 | A | B |
| 107 | 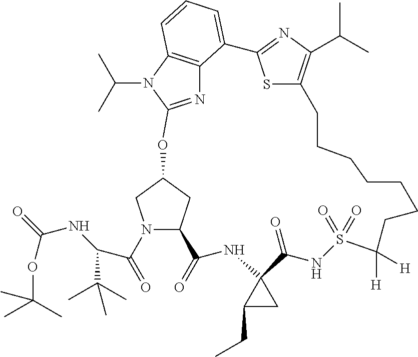 | A | B |
| 108 | 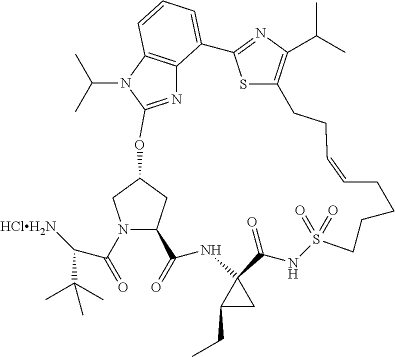 | A | A |
| 109 | 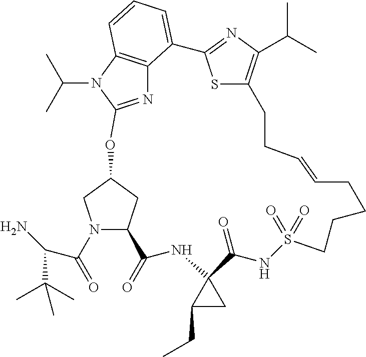 | A | A |
| 110 | 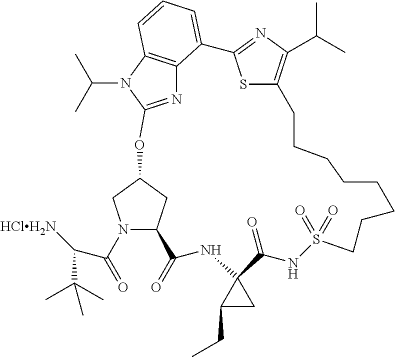 | A | A |
| 111 | 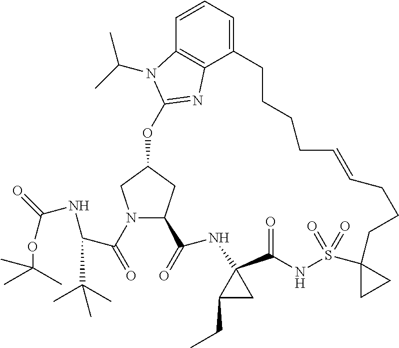 | A | B |
| 112 | 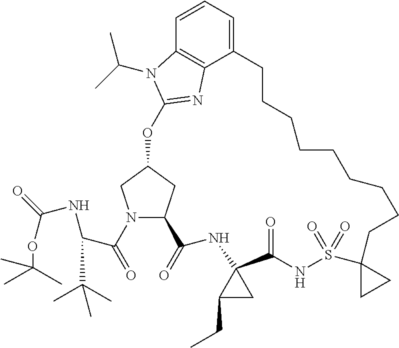 | A | C |
| 113 | 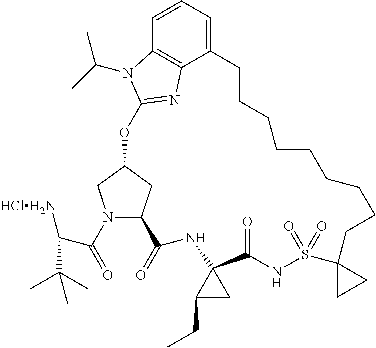 | A | B |

A indicates an EC₅₀ or IC₅₀≧100 nM
B indicates an EC₅₀ or IC₅₀ between 10 and 100 nM
C indicates an EC₅₀ or IC₅₀ of ≦10 nM

The compounds shown below in Table 2 can be prepared by the methods Section II, modified as appropriate.

TABLE 2

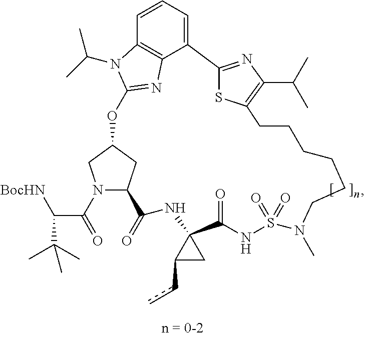

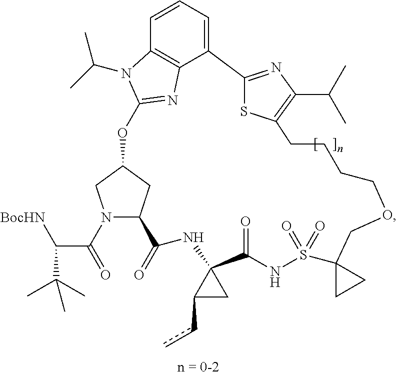

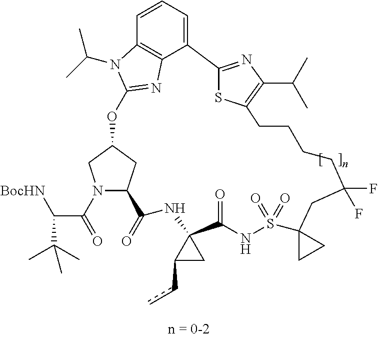

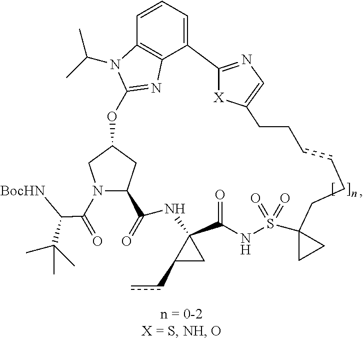

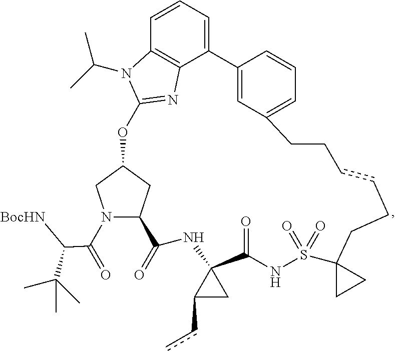

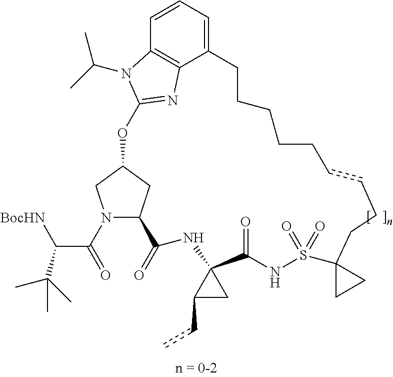

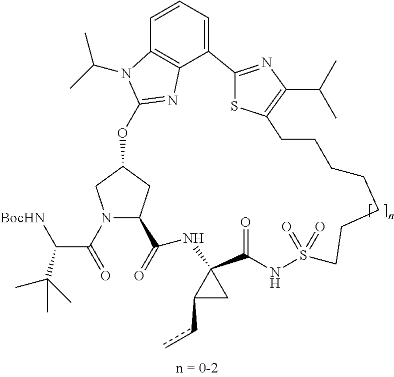

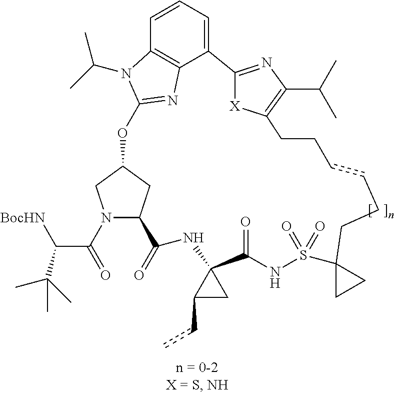

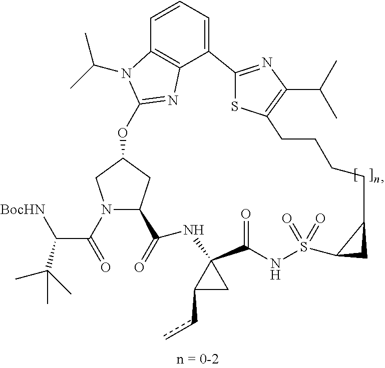

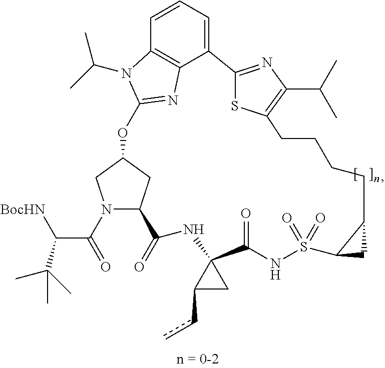

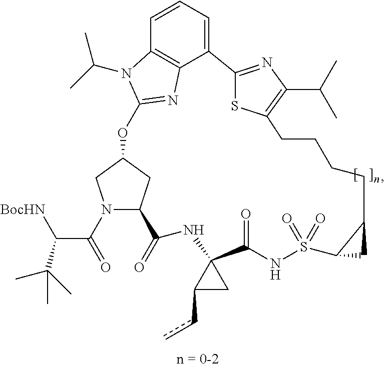

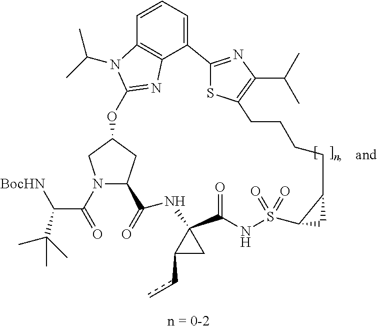

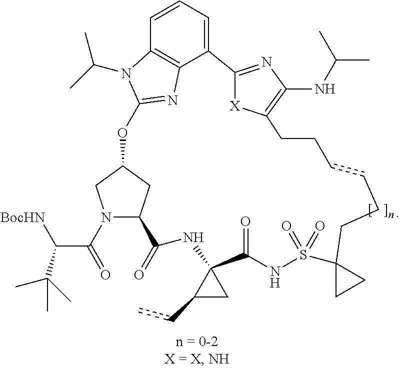

### CONCLUSION

Potent small molecule inhibitors of the HCV NS3 protease have been developed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A compound represented by formula I: 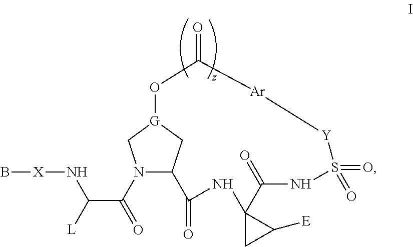
or a pharmaceutically acceptable salt thereof,
wherein Ar is optionally substituted heteroaryl, optionally substituted C₆₋₁₀ aryl, optionally substituted heterocyclyl; or optionally substituted polycyclic moiety;
z is 0 or 1;
G is 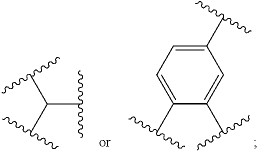
X is a bond, C(═O), CO₂, CONH, SO₂, SO₃, or SO₂NH;
B is H (hydrogen), optionally substituted C₆₋₁₀ aryl, optionally substituted C₂₋₁₀ heteroaryl, or optionally substituted C₁₋₁₀ hydrocarbyl;
L is H (hydrogen) or optionally substituted C₁₋₁₀ hydrocarbyl;
Y is (L₁)ₚ;
p is an integer from 5 to 12;
each L₁ is separately selected, where L₁ is selected from the group consisting of C(R²)₂, NR³, O (oxygen), —(R²)C═C(R²)—, C(═O), C₃₋₇ cycloalkyl, optionally substituted aryl, optionally substituted polycyclic moiety, optionally substituted heterocycle and optionally substituted heteroaryl;
each R² is separately selected, where R² is selected from the group consisting of H (hydrogen), C₁₋₆alkoxy, aryl, halo, hydroxy, R^{a}R^{b}N—, C₁₋₆alkyl optionally substituted with up to 5 halo, and C₁₋₆alkoxy optionally substituted with up to 5 halo, or optionally two vicinal R² and the carbons to which they are attached are together a fused three- to six-membered carbocyclic ring optionally substituted with up to two C₁₋₆alkyl groups, or optionally two geminal R² and the carbon to which they are attached are together a fused three- to six-membered carbocyclic ring optionally substituted with up to two C₁₋₆alkyl groups;
each R^{a}R^{b}N is separately selected, wherein R^{a} and R^{b} are each separately selected from the group consisting of hydrogen, C₂₋₆alkenyl, and C₁₋₆alkyl;
each R³ is separately selected, where R³ is selected from the group consisting of hydrogen, optionally substituted aryl, optionally substituted heteroaryl, and optionally substituted C₁₋₆alkyl; and
E is H (hydrogen) or optionally substituted C₁₋₆ hydrocarbyl, with the proviso that a compound represented by formula I is not selected from the group consisting of:

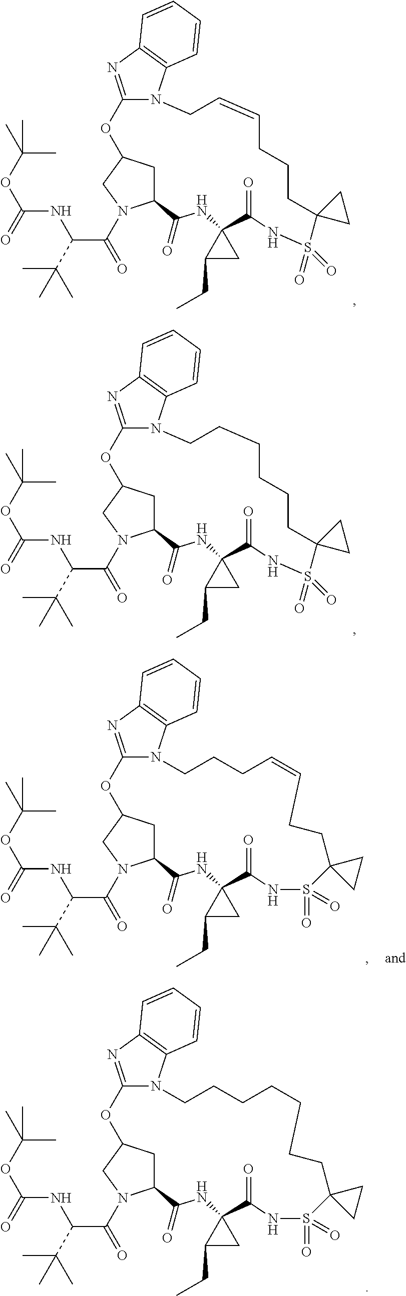

2. The compound of claim 1, wherein z is 0.

3. The compound of claim 1, wherein G is

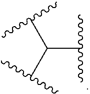

4. The compound of claim 1, wherein Ar is selected from: 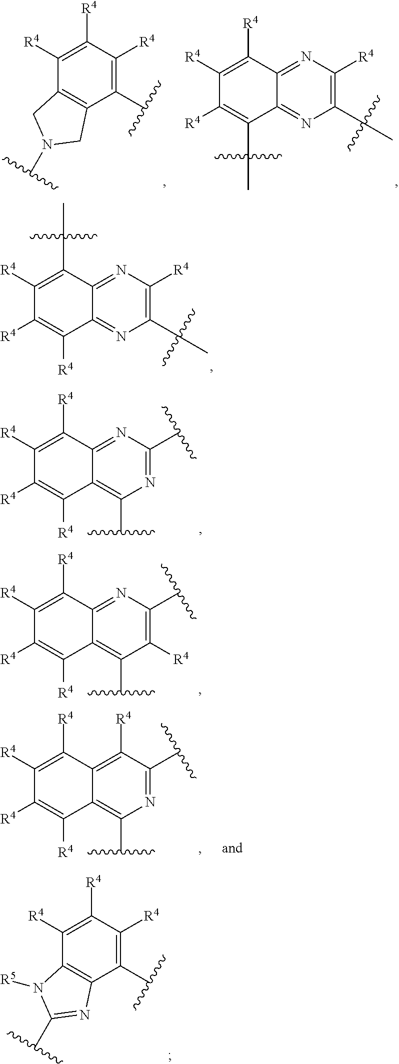
each R⁴ is separately selected, where R⁴ is independently selected from the group consisting of H (hydrogen), halo, C₁₋₆alkyl optionally substituted with up to five fluoro, and C₁₋₆alkoxy optionally substituted with up to five fluoro; and
R⁵ is selected from the group consisting of H (hydrogen), and C₁₋₆alkyl optionally substituted with up to five fluoro.

5. The compound of claim 1, wherein Ar is optionally substituted benzoimidazolen-1,2-yl.

6. The compound of claim 1, wherein X is a bond, C(═O), CO₂, CONH, SO₂, or SO₂NH.

7. The compound of claim 1, wherein X is a bond, C(═O), CO₂, CONH, or SO₂.

8. The compound of claim 1, wherein X is a bond, C(═O), CO₂, or CONH.

9. The compound of claim 1, wherein X is a bond or CO₂.

10. The compound of claim 1, wherein B is H (hydrogen) or C₁₋₆ alkyl.

11. The compound of claim 10, wherein B is H (hydrogen) or t-butyl.

12. The compound of claim 1, wherein E is ethyl.

13. The compound of claim 1, wherein L is t-butyl.

14. The compound of claim 1, wherein X is a bond and B is H (hydrogen).

15. The compound of claim 10, wherein B is ethyl, n-propyl, isopropyl, n-butyl, isobutyl, sec-butyl, t-butyl, or a pentyl isomer; E is methyl, ethyl, propyl, or vinyl; and L is ethyl, n-propyl, isopropyl, n-butyl, isobutyl, sec-butyl, tert-butyl, or a pentyl isomer.

16. The compound of claim 15, wherein B is t-butyl, E is ethyl, and L is t-butyl.

17. The compound of claim 1, wherein Y is represented by: 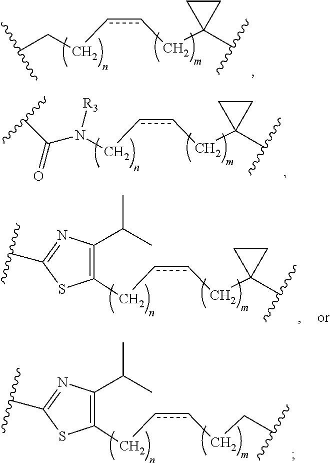
wherein the dashed line represents the presence or absence of a bond and if present, the resulting double bond may be cis or trans; and
m and n are independently 0, 1, 2, 3, 4, 5, or 6.

18. The compound of claim 17, wherein the sum of m and n is 4, 5, 6, or 7.

19. The compound of claim 1, wherein the compound represented by formula I has the structure of formula Ia, Ib, or Ic: 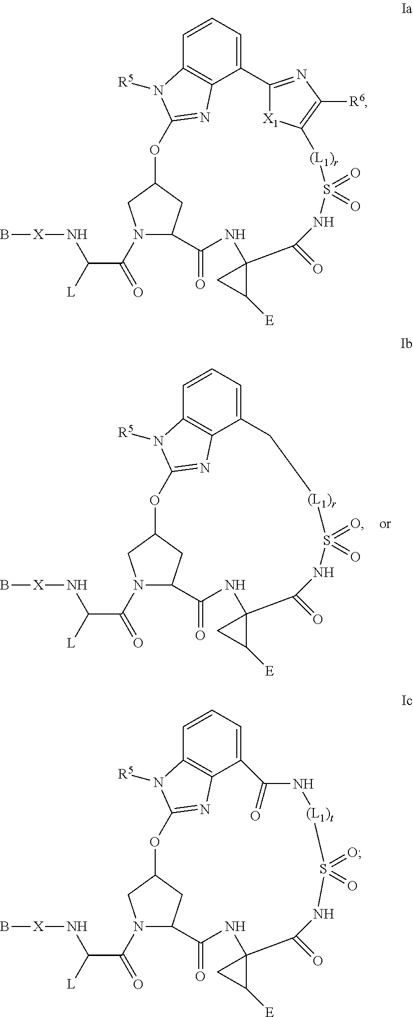
r is an integer from 4 to 8;
t is an integer from 3 to 7;
R⁵ is selected from the group consisting of H (hydrogen), and C₁₋₆alkyl optionally substituted with up to five fluoro;
R⁶ is selected from the group consisting of mono-(C₁-C₆)alkylamino, di-(C₁-C₆)alkylamino, C₁₋₆alkyl optionally substituted with up to five fluoro, optionally substituted arylalkyl, optionally substituted aryl, optionally substituted heteroaryl, optionally substituted heterocyclyl, and optionally substituted polycyclic moiety; and
X₁ is NH, O (oxygen), or S (sulfur).

20. The compound of claim 1, wherein unless otherwise specified, groups indicated as “optionally substituted” are optionally substituted with one or more group(s) individually and independently selected from C₁-C₆ alkyl, C₁-C₆ alkenyl, C₁-C₆ alkynyl, C₃-C₆ cycloalkyl, C₃-C₆ heterocycloalkyl, aryl, heteroaryl, halo, cyano, hydroxy, C₁-C₆ alkoxy, aryloxy, sulfhydryl, C₁-C₆ alkylthio, arylthio, mono- and di-(C₁-C₆)alkyl amino, quaternary ammonium salts, amino(C₁-C₆)alkoxy, hydroxy(C₁-C₆)alkylamino, amino(C₁-C₆)alkylthio, cyanoamino, nitro, carbamyl, oxo, carbonyl, carboxy, sulfamyl, sulfonyl, sulfinyl, thiocarbonyl, and thiocarboxy.

21. A compound having the structure of formula II: 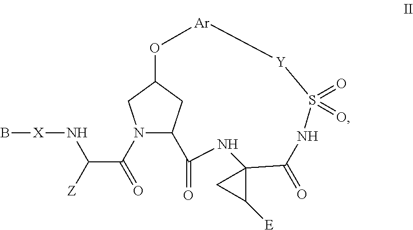
or a pharmaceutically acceptable salt thereof,
wherein Ar is optionally substituted C₅₋₁₀ fused bicyclic heteroaryl, optionally substituted C_{6 or 10} aryl; or optionally substituted polycyclic moiety;
Y is (L₁)ₚ;
p is an integer from 5 to 9;
each L₁ is separately selected, where L₁ is selected from the group consisting of C(R²)₂, NR³, O (oxygen), —(R²)C═C(R²)—, C(═O), C₃₋₇ cycloalkyl, optionally substituted aryl, optionally substituted polycyclic moiety, optionally substituted heterocycle and optionally substituted heteroaryl;
each R² is separately selected, where R² is selected from the group consisting of H (hydrogen), C₁₋₆alkoxy, C₁₋₆alkyl, aryl, halo, hydroxy, R^{a}R^{b}N—,C₁₋₆alkyl optionally substituted with up to 5 halo, and C₁₋₆alkoxy optionally substituted with up to 5 halo, or optionally two vicinal R² and the carbons to which they are attached are together a fused three- to six-membered carbocyclic ring optionally substituted with up to two C₁₋₆alkyl groups, or optionally two geminal R² and the carbon to which they are attached are together a fused three- to six-membered carbocyclic ring optionally substituted with up to two C₁₋₆alkyl groups;
each R^{a}R^{b}N is separately selected, wherein R^{a} and R^{b} are each separately selected from the group consisting of hydrogen, C₂₋₆alkenyl, and C₁₋₆alkyl;
each R³ is separately selected, where R³ is selected from the group consisting of hydrogen, optionally substituted aryl, optionally substituted heteroaryl, optionally substituted C₁₋₆alkyl, and C₁₋₆alkyl optionally substituted with up to 5 halo;
X is a bond, C(═O), —C(═O)O—, —C(═O)NH—, SO₂, SO₃, or SO₂NH;
B is H (hydrogen), optionally substituted C₁₋₆ alkyl, optionally substituted C₂₋₆ alkenyl, optionally substituted C₂₋₆ alkynyl, optionally substituted C₃₋₇ cycloalkyl, optionally substituted C_{6 or 10} aryl, optionally substituted C₅₋₁₀ heteroaryl, or optionally substituted C₅₋₁₀ heterocycle;
Z is H (hydrogen) or optionally substituted C₁₋₁₀ hydrocarbyl; and
E is H (hydrogen) or optionally substituted C₁₋₆ hydrocarbyl, with the proviso that a compound represented by formula II is not selected from the group consisting of:

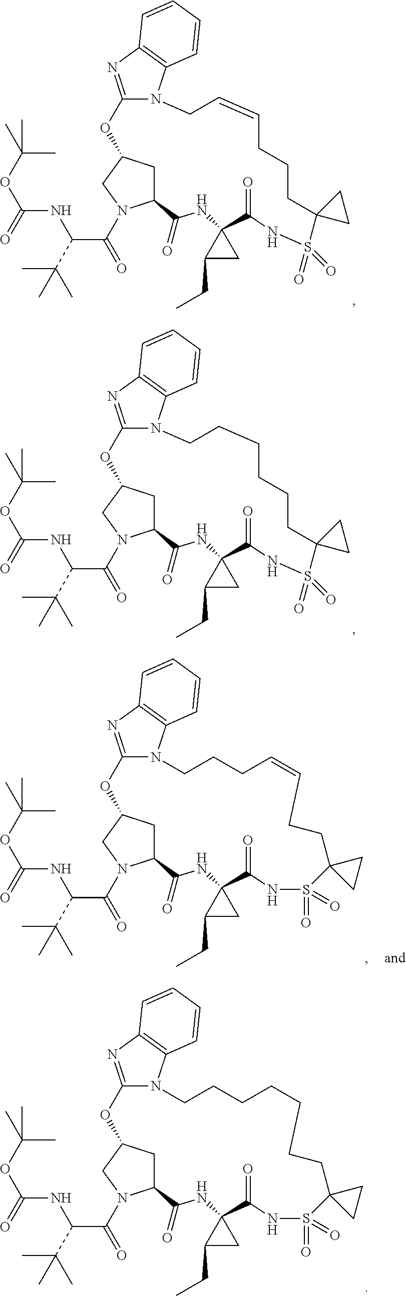

22. The compound of claim 21, wherein Ar is C₅₋₁₀ fused bicyclic heteroaryl, C_{6 or 10} aryl; or polycyclic moiety, each optionally substituted with one or more groups independently selected from the group consisting of halo, C₁₋₆alkyl optionally substituted with up to five fluoro, and C₁₋₆alkoxy optionally substituted with up to five fluoro.

23. The compound of claim 21, wherein Ar is C₅₋₁₀ fused bicyclic heteroaryl, substituted with halo, C₁₋₆alkyl optionally substituted with up to five fluoro, or C₁₋₆alkoxy optionally substituted with up to five fluoro.

24. The compound of claim 21, wherein Ar is C₅₋₁₀ fused bicyclic heteroaryl, substituted with halo or C₁₋₆alkyl optionally substituted with up to five fluoro.

25. The compound of claim 21, wherein Ar is C₅₋₁₀ fused bicyclic heteroaryl, substituted with C₁₋₆alkyl optionally substituted with up to five fluoro.

26. The compound of claim 21, wherein Ar is 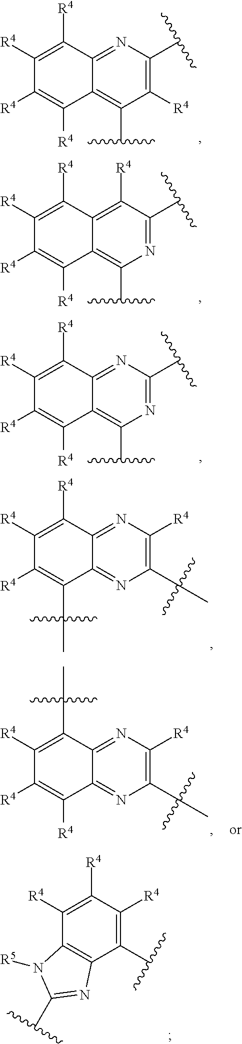
each R⁴ is separately selected, where R⁴ is independently selected from the group consisting of H (hydrogen), halo, C₁₋₆alkyl optionally substituted with up to five fluoro, and C₁₋₆alkoxy optionally substituted with up to five fluoro; and
R⁵ is selected from the group consisting of H (hydrogen), and C₁₋₆alkyl optionally substituted with up to five fluoro.

27. The compound of claim 1,
wherein:
X is —C(═O)O—;
B is optionally substituted C₁₋₆ alkyl.

28. The compound of claim 21,
wherein:
X is a bond;
B is H (hydrogen), or B is C_{6 or 10} aryl, C₅₋₁₀ heteroaryl, or C₅₋₁₀ heterocycle, each optionally substituted with one or more groups independently selected from the group consisting of halo, hydroxy, C₁₋₆alkyl optionally substituted with up to five fluoro, and C₁₋₆alkoxy optionally substituted with up to five fluoro.

29. The compound of claim 21,
wherein Y is selected from the group consisting of 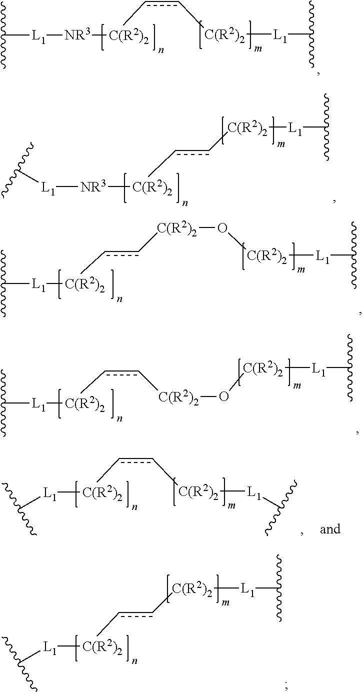
m and n are each independently 0, 1, 2, 3, 4, 5, or 6;
each L₁ is separately selected from the group consisting of C(R²)₂, C(═O), C₃₋₇ cycloalkyl, and optionally substituted heteraryl; and
the dashed line indicates an optional double bond.

30. The compound of claim 29, wherein the sum of m and n is 4, 5, or 6.

31. The compound of claim 29, wherein Y is selected from the group consisting of: 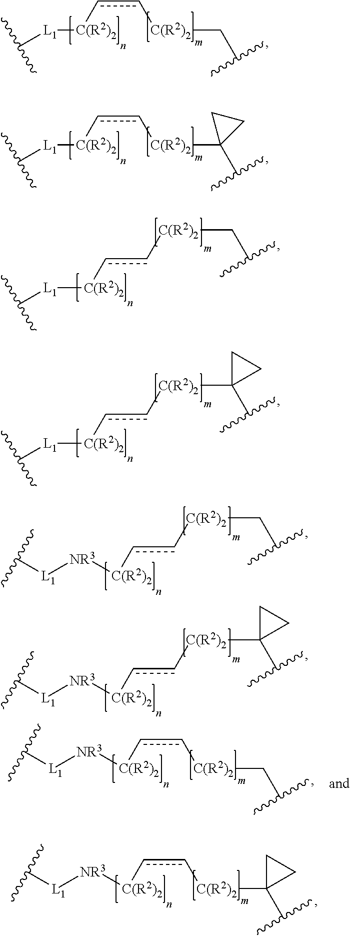
each L₁ is separately selected from the group consisting of CH₂, C(═O), and 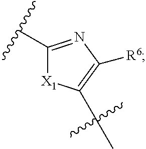
R⁶ is selected from the group consisting of mono-(C₁-C₆)alkylamino, di-(C₁-C₆)alkylamino, C₁₋₆alkyl optionally substituted with up to five fluoro, optionally substituted arylalkyl, optionally substituted aryl, optionally substituted heteroaryl, optionally substituted heterocyclyl, and optionally substituted polycyclic moiety; and
X₁ is NH, O (oxygen), or S (sulfur).

32. The compound of claim 29,
wherein Y is selected from the group consisting of 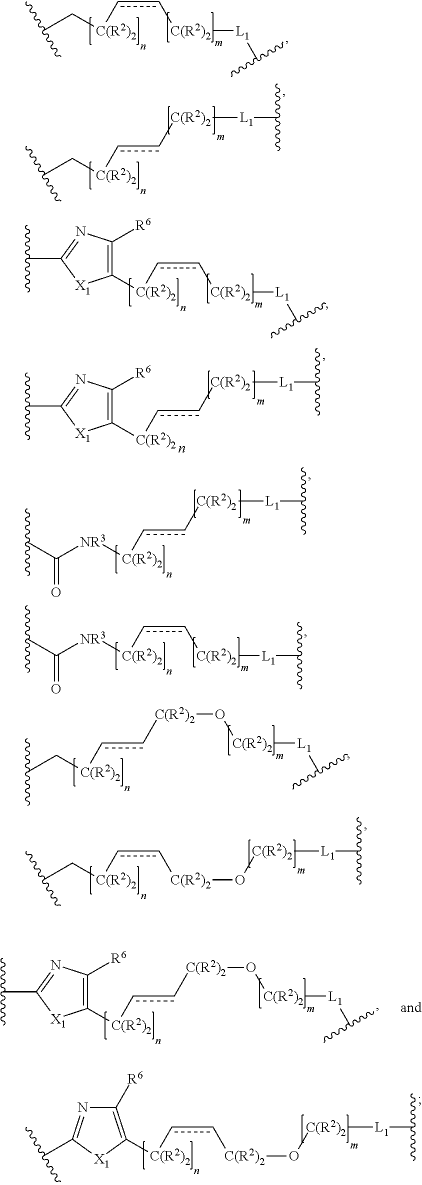
R⁶ is selected from the group consisting of H (hydrogen), halo, C₁₋₆alkyl optionally substituted with up to five fluoro, and C₁₋₆alkoxy optionally substituted with up to five fluoro;
X₁ is NH, O (oxygen), or S (sulfur); and
L₁ is CH₂ or cyclopropyl.

33. The compound of claim 21,
wherein Y is selected from the group consisting of 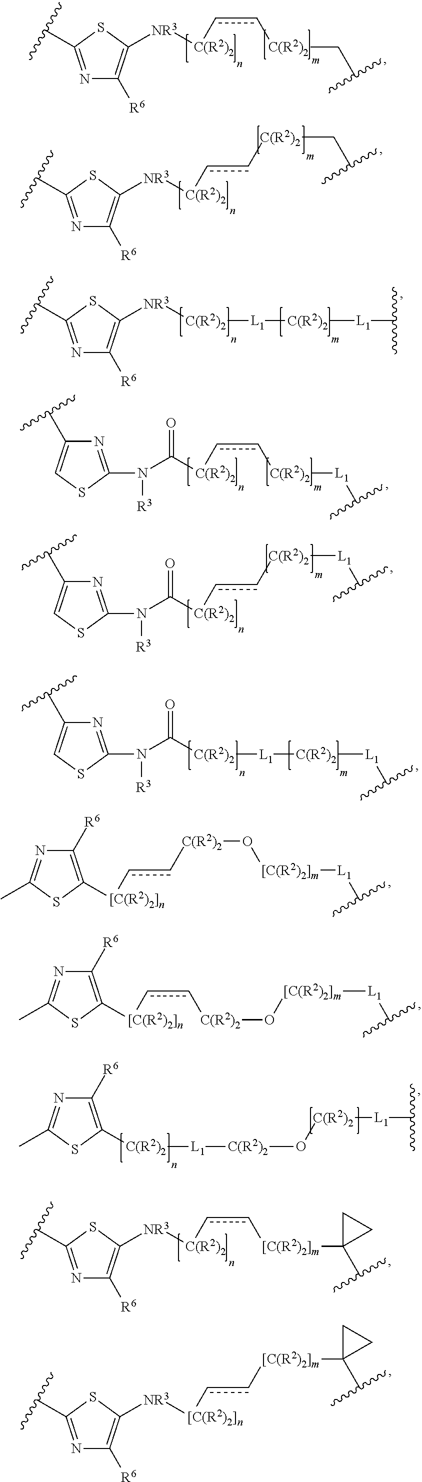 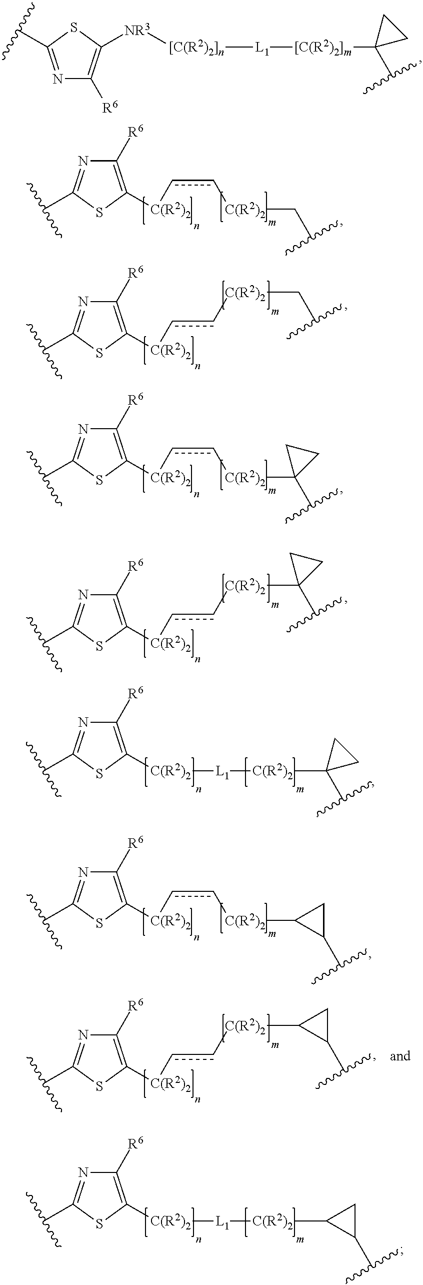
m and n are each independently 0, 1, 2, 3, 4, 5, or 6;
R⁶ is selected from the group consisting of H (hydrogen), halo, C₁₋₆alkyl optionally substituted with up to five fluoro, and C₁₋₆alkoxy optionally substituted with up to five fluoro; and
the dashed line indicates an optional double bond.

34. The compound of claim 21,
wherein Y is selected from the group consisting of 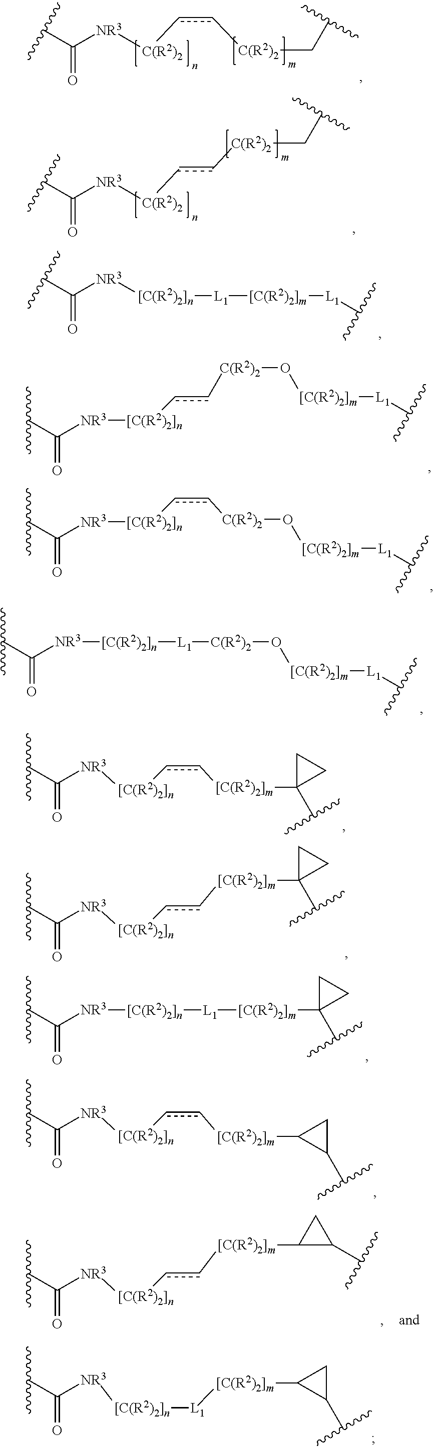
m and n are each independently 0, 1, 2, 3, 4, 5, or 6;
R³ is selected from the group consisting of H (hydrogen), and C₁₋₆alkyl optionally substituted with up to five fluoro; and
the dashed line indicates an optional double bond.

35. The compound of claim 21, wherein Z is C₁₋₆ alkyl; and E is C₁₋₆ alkyl or C₂₋₆ alkenyl.

36. The compound of claim 21, wherein Ar is

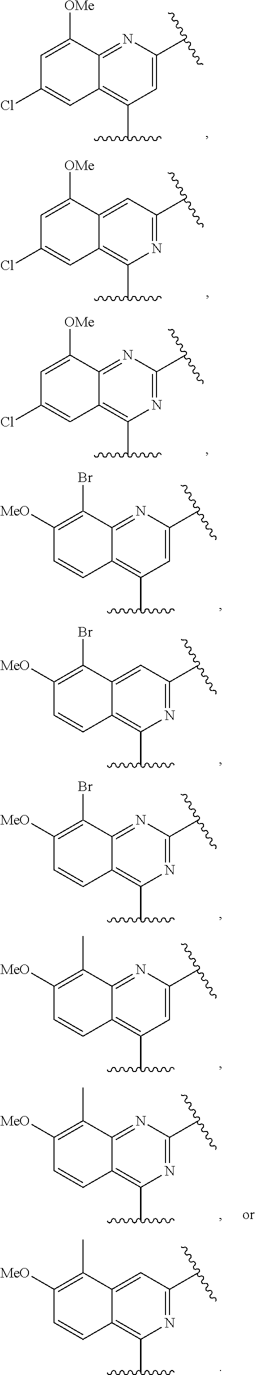

37. The compound of claim 36,
wherein Y is selected from the group consisting of 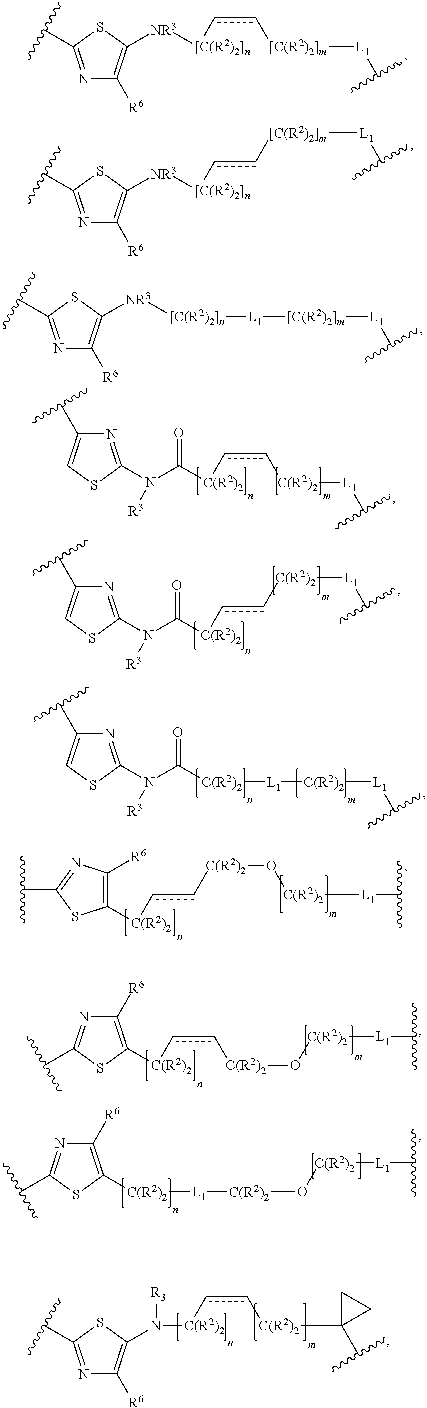 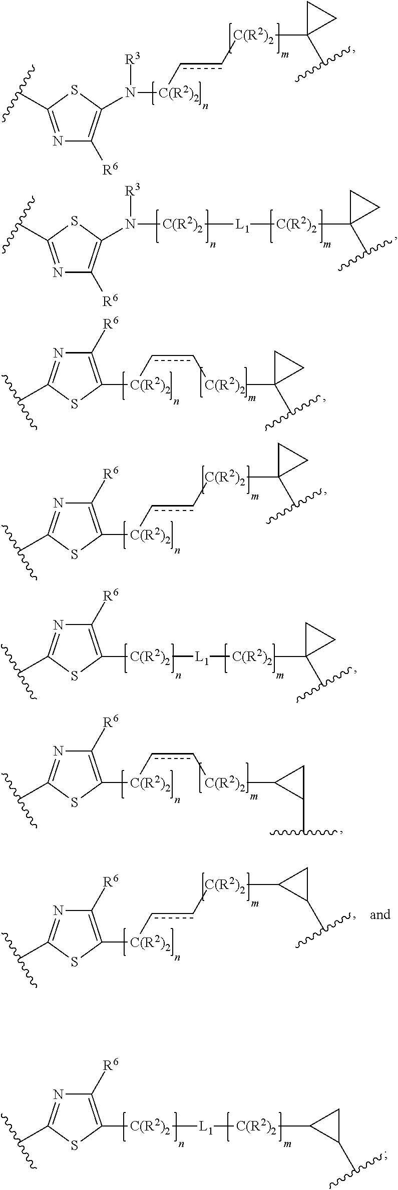
m and n are each independently 0, 1, 2, 3, 4, 5, or 6;
R⁶ is selected from the group consisting of H (hydrogen), halo, C₁₋₆alkyl optionally substituted with up to five fluoro, and C₁₋₆alkoxy optionally substituted with up to five fluoro; and
the dashed line indicates an optional double bond.

38. The compound of claim 36,
wherein Y is selected from the group consisting of 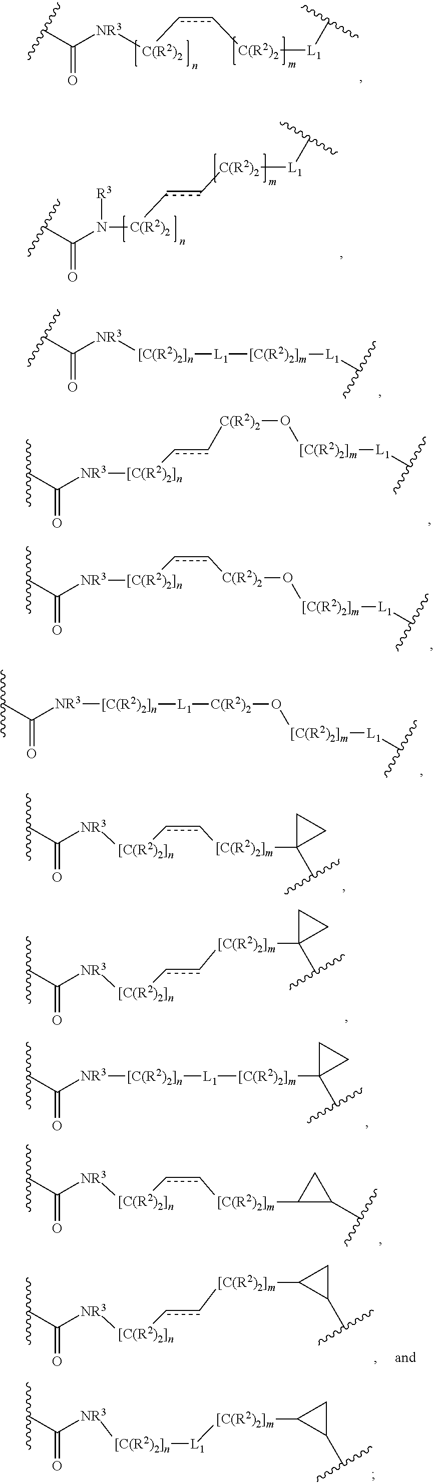
m and n are each independently 0, 1, 2, 3, 4, 5, or 6;
R³ is selected from the group consisting of H (hydrogen), and C₁₋₆alkyl optionally substituted with up to five fluoro; and
the dashed line indicates an optional double bond.

39. The compound of claim 21, wherein the compound having the structure of formula II has the structure of formula IIa: 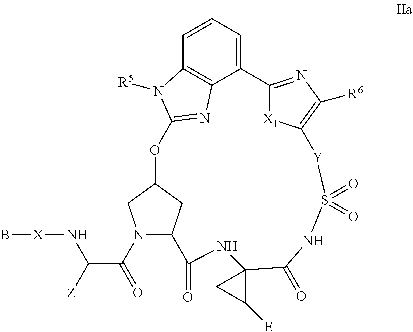
Y is (L₁)ᵣ;
r is an integer from 4 to 8;
R⁵ is selected from the group consisting of H (hydrogen), and C₁₋₆alkyl optionally substituted with up to five fluoro;
R⁶ is selected from the group consisting of mono-(C₁-C₆)alkylamino, di-(C₁-C₆)alkylamino, C₁₋₆alkyl optionally substituted with up to five fluoro, optionally substituted arylalkyl, optionally substituted aryl, optionally substituted heteroaryl, optionally substituted heterocyclyl, and polycyclic moiety; and
X₁ is NH, O (oxygen), or S (sulfur).

40. The compound of claim 21, wherein unless otherwise specified, groups indicated as “optionally substituted” are optionally substituted with one or more group(s) individually and independently selected from C₁-C₆ alkyl, C₁-C₆ alkenyl, C₁-C₆ alkynyl, C₃-C₆ cycloalkyl, C₃-C₆ heterocycloalkyl, aryl, heteroaryl, halo, cyano, hydroxy, C₁-C₆ alkoxy, aryloxy, sulfhydryl, C₁-C₆ alkylthio, arylthio, mono- and di-(C₁-C₆)alkyl amino, quaternary ammonium salts, amino(C₁-C₆)alkoxy, hydroxy(C₁-C₆)alkylamino, amino(C₁-C₆)alkylthio, cyanoamino, nitro, carbamyl, oxo, carbonyl, carboxy, sulfamyl, sulfonyl, sulfinyl, thiocarbonyl, and thiocarboxy.

41. A pharmaceutical composition comprising a pharmaceutically acceptable excipient and a compound of claim 1.

42. A method of treating HCV infection in an individual, the method comprising administering to the individual an effective amount of a compound of claim 1.

43. The method of claim 42, further comprising identifying a subject suffering from a hepatitis C infection.

44. A method of treating liver fibrosis in an individual, the method comprising administering to the individual an effective amount of a compound of a compound of claim 1.

45. The method of claim 44, further comprising identifying a subject suffering from a hepatitis C infection.

46. A method of increasing liver function in an individual having a hepatitis C virus infection, the method comprising administering to the individual an effective amount of a compound of a compound of claim 1.

47. The method of claim 46, further comprising identifying a subject suffering from a hepatitis C infection.
